(19) United States
(12) Patent Application Publication
(10) Pub. No.: US 2025/0121098 A1
(43) Pub. Date: Apr. 17, 2025

(54) BRAIN TARGETED NANOPARTICLES OR CONJUGATES AND METHODS OF USE THEREOF

(71) Applicant: Arizona Board of Regents on behalf of Arizona State University, Scottsdale, AZ (US)

(72) Inventors: Shu Wang, Phoenix, AZ (US); Zhaoyang Fan, Phoenix, AZ (US)

(21) Appl. No.: 18/725,536

(22) PCT Filed: Dec. 21, 2022
(86) PCT No.: PCT/US2022/082116

§ 371 Date: Jun. 28, 2024

Related U.S. Application Data
(60) Provisional application No. 63/294,922, filed on Dec. 30, 2021.

(51) Int. Cl. A61K 49/00; A61K 47/54; A61K 47/69

(52) U.S. Cl. CPC A61K 49/0093; A61K 47/542; A61K 47/544; A61K 47/6911; A61K 47/6929; A61K 49/0084

(57) ABSTRACT

The present invention provides nanoparticles or conjugates comprising at least one ligand that selectively targets major facilitator superfamily domain-containing protein-2a (MFSD2A). In various embodiments, the nanoparticles or conjugates of the invention target at least one cell comprising MFSD2A (e.g., endothelial cells of blood brain barrier). In some embodiments, the nanoparticles or conjugates of the invention cross the blood brain barrier and/or blood retinal barrier. In other aspects, the present invention relates to methods for in vivo delivery of diagnostic and/or therapeutic agents to a brain. In other aspects, the present invention relates to methods of preventing or treating a neurological or cognitive disease or disorder using the nanoparticles or conjugates of the invention.

### CROSS-REFERENCE TO RELATED APPLICATIONS

This application claims priority to U.S. Provisional Application No. 63/294,922, filed Dec. 30, 2021, which is hereby incorporated by reference in its entirety.

### BACKGROUND OF THE INVENTION

The brain represents about 2% of total body weight, but consumes about 20% of total oxygen. The total length of human brain blood vessels is about 600 kilometers, which penetrates into all areas of the brain. However, only about 2% of small molecules in the blood can penetrate through the blood brain barrier (BBB) into the brain. Most of them require transporters on the BBB. This results in ineffective drug therapy of many diseases including neurodegenerative diseases, opioid overdose, disorders of food intake, temperature regulation and mood.

Thus, there is a need in the art for carriers with greater efficiency and lesser toxicity that can cross the BBB or blood retinal barrier (BRB) to deliver therapeutic agents and diagnostic agents to the brain. The present invention satisfies this unmet need.

### SUMMARY OF THE INVENTION

In one aspect, the present invention relates, in part, to a nanoparticle comprising at least one ligand that selectively targets major facilitator superfamily domain-containing protein-2a (MFSD2A). In another aspect, the present invention relates, in part, to a conjugate comprising at least one ligand that selectively targets MFSD2A. In some embodiments, the ligand is selected from acyl-carnitine, fatty acid-lysophosphatidylcholine (LysoPC), docosahexaenoic acid LysoPC (DHA-LysoPC), eicosapentaenoic acid LysoPC (EPA-LysoPC), linoleic acid LysoPC (LA-LysoPC), linolelaidic acid LysoPC, tetradecenoyl-carnitine, palmitoyl-carnitine, 3-hydroxypalmitoyl-carnitine, EPA-carnitine, DHA-carnitine, lysophosphatidic acid (LysoPA), lysophosphatidylethanolamine (LysoPE), lysophosphatidylinositol (LysoPI), lysophosphatidylserine (LysoPS), or any combination thereof.

In some embodiments, the nanoparticle or the conjugate inhibits or reduces the activity of MF SD2A.

In some embodiments, the nanoparticle or the conjugate selectively targets at least one cell comprising MFSD2A. In one embodiment, at least one cell comprising MFSD2A is an endothelial cell. In one embodiment, the endothelial cell is an endothelial cell of a blood brain barrier.

In some embodiments, the nanoparticle or the conjugate crosses the blood brain barrier, blood retinal barrier, or a combination thereof.

In some embodiments, the nanoparticle or the conjugate further comprises at least one agent. In some embodiments, the agent is encapsulated within, adhered to a surface of, or integrated into the structure of said nanoparticle and/or conjugate. In some embodiments, the agent is selected from a small molecule, a protein, a nucleic acid molecule, an antibody, a diagnostic agent, an imaging agent, a therapeutic agent, or any combination thereof. In some embodiments, the nucleic acid molecule is selected from DNA, cDNA, RNA, mRNA, miRNA, siRNA, modified RNA, microRNA, antagomir, antisense molecule, targeted nucleic acid, CRISPR-Cas9 guide RNA, or any combination thereof.

In one aspect, the present invention relates, in part, to a composition comprising at least one nanoparticle and/or at least one conjugate described herein.

In one aspect, the present invention relates, in part, to a method of delivering an agent to a subject in need thereof.

In another aspect, the present invention relates, in part, to a method of treating or preventing at least one disease or disorder in a subject in need thereof.

In another aspect, the present invention relates, in part, to a method of diagnosing a disease or disorder in a subject in need thereof.

In some embodiments, the method comprises administering a therapeutically effectively amount of the composition comprising at least one nanoparticle and/or at least one conjugate described herein to the subject. In one embodiment, the composition comprises at least one diagnostic agent.

In some embodiments, the disease or disorder is selected from a brain disease or disorder, neurological disease or disorder, cognitive disease or disorder, brain tumor, neurodegenerative disease, food intake disorder, schizophrenia, depression, addiction disease or disorder, or any combination thereof.

### BRIEF DESCRIPTION OF THE DRAWINGS

The following detailed description of the invention will be better understood when read in conjunction with the appended drawings. For the purpose of illustrating the invention, there are shown in the drawings specific embodiments. It should be understood, however, that the invention is not limited to the precise arrangements and instrumentalities of the embodiments shown in the drawings.

FIG. 1, comprising FIG. 1A and FIG. 1B, depicts exemplary results demonstrating that major facilitator superfamily domain-containing protein-2a (MFSD2A)-targeted nanoparticles (L-NPs) target more efficiently to the brain than non-targeted nanoparticles (V-NPs). FIG. 1A depicts exemplary images of nanoparticle-encapsulated dye intensity in the brain of mice 3 hours after injection with dye-loaded V-NPs or L-NPs. FIG. 1B depicts exemplary images of nanoparticle-encapsulated dye intensity in the brain of mice 16 hours after injection with dye-loaded V-NPs or L-NPs.

FIG. 2, comprising FIG. 2A through FIG. 2C, depicts exemplary results demonstrating that MFSD2A-targeted lipid nanoparticles (L-NPs) loaded with phytochemicals target more efficiently to the brain than non-targeted nanoparticles (V-NPs). FIG. 2A depicts exemplary images of nanoparticle-encapsulated dye intensity in the brain of mice 20 hours after injection with dye-loaded V-NPs or L-NPs. FIG. 2B depicts exemplary images of nanoparticle-encapsulated dye intensity in the abdomen of mice 20 hours after injection with dye-loaded V-NPs or L-NPs. FIG. 2C depicts exemplary images of nanoparticle-encapsulated dye intensity in brains isolated from mice 20 hours after injection with dye-loaded V-NPs or L-NPs.

FIG. 3, comprising FIG. 3A through FIG. 3D, depicts exemplary results demonstrating that MFSD2A-targeted liposomes carrying lysophosphatidyl-DHA (LDLs) target more efficiently to the brain than non-targeted liposomes (VLs). FIG. 3A depicts exemplary images of liposome-encapsulated dye intensity in the brain of mice 1 hour after injection with dye-loaded LDLs or VLs. FIG. 3B depicts exemplary images of liposome-encapsulated dye intensity in the abdomen of mice 1 hour after injection with dye-loaded LDLs or VLs. FIG. 3C depicts exemplary images of liposome-encapsulated dye intensity in the brain of mice 18 hours after injection with dye-loaded LDLs or VLs. FIG. 3D depicts exemplary images of liposome-encapsulated dye intensity in the abdomen of mice 18 hours after injection with dye-loaded LDLs or VLs.

FIG. 4, comprising FIG. 4A and FIG. 4B, depicts exemplary results demonstrating that MFSD2A-targeted liposomes carrying lysophosphatidyl-DHA (LDLs) target more efficiently to the brain than non-targeted liposomes (VLs). FIG. 4A depicts exemplary images of nanoparticle-encapsulated dye intensity in brains isolated from mice 18 hours after injection with dye-loaded LDLs or VLs. FIG. 4B depicts exemplary images of nanoparticle-encapsulated dye intensity in various organs isolated from mice 18 hours after injection with dye-loaded LDLs or VLs.

FIG. 5, comprising FIG. 5A through FIG. 5D, depicts exemplary results demonstrating that MFSD2A-targeted liposomes carrying lysophosphatidyl-DHA (LDLs) target more efficiently to the brain than non-targeted liposomes (VLs). FIG. 5A depicts exemplary images of liposome-encapsulated dye intensity in the brain of mice 3 hours after injection with dye-loaded LDLs or VLs. FIG. 5B depicts exemplary images of liposome-encapsulated dye intensity in the abdomen of mice 3 hours after injection with dye-loaded LDLs or VLs. FIG. 5C depicts exemplary images of liposome-encapsulated dye intensity in the brain of mice 24 hours after injection with dye-loaded LDLs or VLs. FIG. 5D depicts exemplary images of liposome-encapsulated dye intensity in the abdomen of mice 24 hours after injection with dye-loaded LDLs or VLs.

FIG. 6, comprising FIG. 6A through FIG. 6D, depicts exemplary results demonstrating that MFSD2A-targeted liposomes carrying lysophosphatidyl-DHA (LDLs) target more efficiently to the brain than non-targeted liposomes (VLs). FIG. 6A depicts exemplary images of nanoparticle-encapsulated dye intensity in brains isolated from mice 24 hours after injection with dye-loaded LDLs or VLs. FIG. 6B depicts exemplary images of nanoparticle-encapsulated dye intensity in various organs isolated from mice 24 hours after injection with dye-loaded LDLs or VLs. FIG. 6C depicts exemplary images of nanoparticle-encapsulated dye intensity in eyes isolated from mice 24 hours after injection with dye-loaded LDLs or VLs.

FIG. 6D depicts exemplary images of nanoparticle-encapsulated dye intensity in the body of mice after the brain and eyes had been removed 24 hours after injection with dye-loaded LDLs or VLs.

FIG. 7, comprising FIG. 7A and FIG. 7B, depicts exemplary results demonstrating that MFSD2A-targeted liposomes carrying lysophosphatidyl-DHA (LDLs) target more efficiently to the brain in older animals than non-targeted liposomes (VLs). FIG. 7A depicts exemplary images of nanoparticle-encapsulated dye intensity in brains isolated from older mice 1 hour after injection with dye-loaded LDLs or VLs. FIG. 7B depicts exemplary images of nanoparticle-encapsulated dye intensity in brains isolated from older mice 24 hours after injection with dye-loaded LDLs or VLs.

FIG. 8, comprising FIG. 8A through FIG. 8D, depicts exemplary results demonstrating that MFSD2A-targeted liposomes carrying lysophosphatidyl-DHA (LDLs) target more efficiently to the brain in older animals than non-targeted liposomes (VLs). FIG. 8A depicts exemplary images of nanoparticle-encapsulated dye intensity in brains isolated from older mice 24 hours after injection with dye-loaded LDLs or VLs. FIG. 8B depicts exemplary images of nanoparticle-encapsulated dye intensity in various organs isolated from older mice 24 hours after injection with dye-loaded LDLs or VLs. FIG. 8C depicts exemplary images of nanoparticle-encapsulated dye intensity in eyes isolated from older mice 24 hours after injection with dye-loaded LDLs or VLs. FIG. 8D depicts exemplary images of nanoparticle-encapsulated dye intensity in the body of older mice after the brain and eyes had been removed 24 hours after injection with dye-loaded LDLs or VLs.

### DETAILED DESCRIPTION

The present invention is based, in part, on the unexpected discovery of nanoparticles, comprising at least one ligand that selectively targets MFSD2A, are capable of crossing the blood brain barrier (BBB) and/or blood retinal barrier. Thus, in one aspect, the present invention provides a nanoparticle that selectively targets at least one cell comprising MFSD2A (e.g., endothelial cells of the BBB). In another aspect, the present invention provides a conjugate that selectively targets at least one cell comprising MFSD2A (e.g., endothelial cells of the BBB). In some embodiments, the nanoparticle or the conjugate of the invention crosses the BBB and/or blood retinal barrier (BRB). In some embodiments, the nanoparticle or the conjugate comprises a diagnostic agent, imaging agent, therapeutic agent, or any combination thereof.

In one aspect, the present invention relates to a method for in vivo delivery of a diagnostic agent, imaging agent, therapeutic agent, or any combination thereof to a brain using at least one nanoparticle and/or at least one conjugate of the invention. In one aspect, the present invention relates to a method of preventing or treating a neurological or cognitive disease or disorder or a brain disease or disorder (e.g. brain tumor, neurodegenerative disease, food intake disorder, schizophrenia, depression, addiction, etc.) using at least one nanoparticle and/or at least one conjugate of the invention. In one aspect, the present invention relates to a method of regulating a condition associated with the brain (e.g., opioids overdose, temperature regulation, mood, etc.) using at least one nanoparticle and/or at least one conjugate of the invention.

### Definitions

Unless defined otherwise, all technical and scientific terms used herein have the same meaning as commonly understood by one of ordinary skill in the art to which this invention belongs. Any methods and materials similar or equivalent to those described herein can be used in the practice or testing of the present invention.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value, for example numerical values and/or ranges, such as an amount, a temporal duration, and the like, is meant to encompass variations of 20%, ±10%, ±5%, ±1%, or ±0.1% from the specified value, as such variations are appropriate to perform the disclosed methods. For example, “about 40 [units]” may mean within ±25% of 40 (e.g., from 30 to 50), within ±20%, ±15%, ±10%, 9%, 8%, ±7%, ±6%, ±5%, ±4%, ±3%, ±2%, ±1%, less than ±1%, or any other value or range of values therein or therebelow. Furthermore, the phrases “less than about [a value]” or “greater than about [a value]” should be understood in view of the definition of the term “about” provided herein.

As used herein, the term “nanoparticle” refers to particles having a particle size on the nanometer scale (e.g., about 1 nm-10,000 nm). For example, the nanoparticle may have a particle size up to about 2,000 nm. In another example, the nanoparticle may have a particle size up to about 100 nm. In another example, the nanoparticle may have a particle size up to about 6 nm. As used herein, “nanoparticle” refers to a number of nanoparticles, including, but not limited to, liposomes, lipid nanoparticles, polymer nanoparticles, organic nanoparticles, inorganic nanoparticles, biocompatible nanoparticles, such as biocompatible organic nanoparticles, biocompatible inorganic nanoparticles, etc., nanoclusters, nanocapsules, core-shell nanocapsules, nanovesicles, micelles, block copolymer micelles, lamaellae shaped particles, polymerosomes, dendrimers, emulsions, exosomes, self-emulsifying drug delivery systems (SEDDS), microspheres, micro-structured lipid carriers, nano-structured lipid carriers, and other nano-size particles of various other small fabrications that are known to those of skill in the art. The shapes and compositions of nanoparticles may be guided during condensation of atoms by selectively favoring growth of particular crystal facets to produce spheres, rods, wires, discs, cages, core-shell structures and many other shapes. The definitions and understandings of the entities falling within the scope of nanocapsule are known to those of skill in the art, and such definitions are incorporated herein by reference and for the purposes of understanding the general nature of the subject matter of the present application. However, the following discussion is useful as a further understanding of some of these terms.

As used herein, the phrase “lipid nanoparticle” refers to a transfer vehicle comprising one or more lipids (e.g., cationic lipids, non-cationic lipids, and PEG-modified lipids) and/or one or more polymers. Examples of suitable lipids include, but are not limited to, the phosphatidyl compounds (e.g., phosphatidylglycerol, phosphatidylcholine, phosphatidylserine, phosphatidylethanolamine, sphingolipids, cerebrosides, and gangliosides). Examples of suitable polymers include, but are not limited to, polyacrylates, polyalkycyanoacrylates, polylactide, polylactide-polyglycolide copolymers, polycaprolactones, dextran, albumin, gelatin, alginate, collagen, chitosan, cyclodextrins, dendrimers, and polyethyleneimine.

The term “liposome” as used herein refers to microscopic vesicles or particles made up of one or more lipid bilayers enclosing an internal aqueous medium. To form liposomes, the presence of at least one “vesicle-forming lipid” is needed, which is an amphipathic lipid capable of either forming or being incorporated into a lipid bilayer. Any suitable vesicle-forming lipid may be used to form the lipid bilayer constituting the liposomes. Vesicle-forming lipid includes, but not limited to, phospholipids such as phosphatidylcholine (PC), phosphatidylglycerol (PG), phosphatidylinositol (PI), phosphatidic acid (PA), phosphatidylethanolamine (PE) or phosphatidylserine (PS), and charged lipids, such as a positively charge lipid or a negatively charged lipid. The term “liposome” may have the same meaning and may be interchanged with the term “lipid vesicle”. Liposome examples include large unilamellar vesicles, multilamellar vesicles, paucilamellar vesicles, small unilamellar vesicles, reverse phase evaporation vesicles, French press vesicles, and ether injection vesicles. Products incorporating liposomes include, but are not limited to, adjuvants, drug carriers, and cleansers.

For example, the term “nanocapsule” refers to a vesicular system or hollow particle with a shell surrounding a core-forming space, which, in certain instances, can be used for transporting a payload on a nanoscale level. A nanocapsule may also be a nano-sized version of a container. The payload of the nanocapsule can be, but is not limited to drugs, medicaments, pharmaceutical compositions, chemical compositions, therapeutic compositions, biological macromolecules, dyes, biological material, immunological compositions, nutritional compositions, vitamins, proteins, nucleic acids, antibodies, and vaccines. Various materials may be used for producing such nanocapsules. Nanocapsule refers to a particle having a hollow core that is surrounded by a shell, such that the particle has a size of less than about 1000 nanometers. When a nanocapsule includes a bioactive component, the bioactive component is located in the core that is surrounded by the shell of the nanocapsule.

As used herein, the term “nanocage” refers to a nanocapsule, whereby the shell is not solid, as described for the nanocapsule, but has multiple holes or pores in its shell, thereby making it possible for the payload within the core of the nanocage to come into contact with the surrounding environment. These holes or pores may be regular or irregular in shape and/or spacing on the surface of the particle.

The term “micelle”, a useful article in the employment of a general aspect of the present invention, can generally be thought of as a small—on the order of usually nanometers in diameter—aggregate of amphiphilic linear molecules having a polar, or hydrophilic end and an opposite non-polar, or hydrophobic end. These linear molecules can be comprised of simple molecules, or polymeric chains. A micelle can also be referred to as an aggregate of surfactant molecules dispersed in a liquid colloid. A typical micelle in aqueous solution can form an aggregate with the hydrophilic “head” regions in contact with surrounding solvent, and the sequestering of the hydrophobic tail regions in the micelle center. Other and similar definitions, descriptions and understandings of micelles are also known to those of skill in the art and are incorporated herein by reference.

The term “polymersome” as used herein refers to a vesicle-type which is typically composed of block copolymer amphiphiles, i.e., synthetic amphiphiles that have an amphiphilicity similar to that of lipids. By virtue of their amphiphilic nature (having a more hydrophilic block (head) and a more hydrophobic block (tail)), the block copolymers are capable of self-assembling into a head-to-tail and tail-to-head bilayer structure similar to liposomes. Compared to liposomes, polymersomes have much larger molecular weights, with number average molecular weights typically ranging from 1,000 to 100,000, from 2,500 to 50,000 or from 5,000 to 25,000, are typically chemically more stable, less leaky, less prone to interfere with biological membranes, and less dynamic due to a lower critical aggregation concentration. These properties result in less opsonization and longer circulation times. The terms “more hydrophilic” and “more hydrophobic” as used in the context of the amphophilic nature of the block copolymers are used in a relative sense. i.e., both can be either hydrophilic or hydrophobic, as long as the difference in polarity between the blocks is sufficient for the formation of polymersomes according to the present invention. In view of the creation of a cavity in which water may be incorporated, in certain aspects the more hydrophilic end of the polymer is to be hydrophilic per se. Further, in view of the use as a therapeutic agent carrier, it is desired that also hydrophobic and/or hydrophilic therapeutic agents can be incorporated into the polymersomes. In one embodiment, the hydrophobic end of the polymer is hydrophobic per se. In one embodiment, the amphiphilic nature of the block copolymers is realized in the form of a block copolymer comprising a block made up of more hydrophilic monomeric units (A) and a block made up of more hydrophobic units (B), the block copolymer having the general structure AₙBₘ, with n and m being integers of from 5 to 5,000, 10 to 1,000, or 10 to 500. It is also conceivable that one or more further units or blocks are built-in, e.g., a unit C with an intermediate hydrophilicity so as to yield a terpolymer having the general structure AₙCₚBₘ, with n and m being as defined above, and p being an integer of from 5 to 5,000, 10 to 1,000, or 10 to 500. Any of the blocks can itself be a copolymer, i.e., comprise different monomeric units of the required hydrophilic respectively hydrophobic nature. In one embodiment, the blocks themselves are homopolymeric. Any of the blocks, in particular the more hydrophilic block, may bear charges. The number and type of charges may depend on the pH of the environment. Any combination of positive and/or negative charges on any of the blocks is contemplated by the present invention.

“Dendrimers” have descriptions, definitions and understandings in the literature. For example, and without limitation and including other and similar definitions, descriptions and understandings in the art, the term dendrimer from the Greek word, “dendron”, for tree, can refer to a synthetic, three-dimensional molecule with branching parts.

“Lamella” is a term whose definitions, descriptions and understandings are also known to those of skill in the art and which are incorporated herein by reference. In a very general sense, lamella or lamellae refers to plate-like, gill-shaped or other layered structures.

The definitions, descriptions and understandings of “nanovesicle” are well known to those of skill in the art and are incorporated herein by reference. For example, “nanovesicle” can refer to a variety of small sac, sac-like or globular structures capable of containing fluid or other material therein.

As used herein, the term “exosome” refers to a subset of circulating microvesicles that are preformed microvesicles that are released from the cell following the exocytic fusion of intracellular multivesicular bodies with the plasma membrane, i.e., exosomes have an endocytic origin. As used herein, it is not intended that an exosome of the invention be limited by any particular size or size range. For example, exosome include a variety of nanoparticles, including microvesicles, epididimosomes, argosomes, exosome-like vesicles, microparticles, promininosomes, prostasomes, dexosomes, texosomes, dex, tex, archeosomes and oncosomes. Exosomes are secreted by a wide range of cells, such as mammalian cells, and are secreted under both normal and pathological conditions. Exosomes, in some embodiments, function as intracellular messengers by virtue of carrying mRNA or other contents from a first cell to another cell (or plurality of cells). In some embodiments, exosomes are involved in blood coagulation, immune modulation, metabolic regulation, cell division, and other cellular processes. Because of the wide variety of cells that secret exosomes, in several embodiments, exosome preparations can be used as a diagnostic tool (e.g., exosomes can be isolated from a particular tissue, evaluated for their nucleic acid or protein content, which can then be correlated to disease state or risk of developing a disease).

The term “emulsion” as used herein refers to a mixture of two or more substances, such as liquids, that are normally immiscible, in which one substance forms droplets that are dispersed within another substance. One substance (the dispersed phase) is dispersed in the other (the continuous phase). Depending on the substances used, the droplets of an emulsion may be in the range of 1 nm to 100 m, e.g., 1 nm to 100 nm, 1 m to 50 m, etc. For example, in one embodiment, the continuous phase is an aqueous phase, and the dispersed phase is an organic (oily or hydrophobic) phase; that is, the emulsion is an oil-in-water emulsion.

As used herein, the term “self-emulsifying drug delivery systems (SEDDS)” refers to isotropic mixtures, consisting of oils, surfactants, and/or cosolvents. In some embodiments, SEDDS increase solubility and bioavailability of poorly soluble drugs. For example, in some embodiments, designed SEDDS formulations are used to improve the oral absorption of highly lipophilic compounds.

As used herein, the term “conjugate” means a product formed by covalent or non-covalent linkage of the ligand that selectively targets MFSD2A to an agent, such as a drug, lipid, polymer, peptide, polypeptide, polypeptide variant, etc.

As used herein, the term “polymorph” refers to crystalline forms having the same chemical composition but different spatial arrangements of the molecules, atoms, and/or ions forming the crystal.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule but retains the essential properties of the reference molecule. An analog or derivative may change its interaction with certain other molecules relative to the reference molecule. An analog or derivative molecule may also include a salt, an adduct, tautomer, isomer, prodrug, or other variant of the reference molecule.

As used herein, the term “prodrug” refers to an agent that is converted into the parent drug in vivo. For example, the term “prodrug” refers to a derivative of a known direct acting drug, which derivative has enhanced delivery characteristics and therapeutic value as compared to the drug, and is transformed into the active drug by an enzymatic or chemical process. In some embodiments, “prodrug” refers to an inactive or relatively less active form of an active agent that becomes active by undergoing a chemical conversion through one or more metabolic processes. In one embodiment, upon in vivo administration, a prodrug is chemically converted to the biologically, pharmaceutically, or therapeutically active form of the compound. In another embodiment, a prodrug is enzymatically metabolized by one or more steps or processes to the biologically, pharmaceutically, or therapeutically active form of the compound. For example, the present compounds can be administered to a subject as a prodrug that includes an initiator bound to an active agent, and, by virtue of being degraded by a metabolic process, the active agent is released in its active form.

The term “tautomers” are constitutional isomers of organic compounds that readily interconvert by a chemical process (tautomerization).

The term “isomers” or “stereoisomers” refers to compounds, which have identical chemical constitution, but differ with regard to the arrangement of the atoms or groups in space.

As used herein, the term “stabilizers” refers to either, or both, primary particle and/or secondary stabilizers, which may be polymers or other small molecules. Non-limiting examples of primary particle and/or secondary stabilizers for use with the present invention include, e.g., starch, modified starch, and starch derivatives, gums, including but not limited to polymers, polypeptides, albumin, amino acids, thiols, amines, carboxylic acid, and any combinations or derivatives thereof. Other examples include, but are not limited to, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya, and biosynthetic gum. Other examples of useful primary particle and/or secondary stabilizers include polymers, such as polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(mides), poly(benzimidazoles), collodion, regenerated proteins, semi-solid cross-linked poly(vinylpyrrolidone).

The terms “coat,” “coated,” or “coating,” as used herein, refer to at least a partial coating of the organic liquid. One hundred percent coverage is not necessarily implied by these terms.

The term “label” when used herein refers to a detectable compound or composition that is conjugated directly or indirectly to a probe to generate a “labeled” probe. The label may be detectable by itself (e.g., radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

“Pharmaceutically acceptable” refers to those properties and/or substances, which are acceptable to the subject from a pharmacological/toxicological point of view and to the manufacturing pharmaceutical chemist from a physical/chemical point of view regarding composition, formulation, stability, subject acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art.

The term “pharmaceutically acceptable salt” refers to any pharmaceutically acceptable salt, which upon administration to the subject is capable of providing (directly or indirectly) a compound as described herein. In some embodiments, such salts are acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts, such as, for example, acetate, trifluoroacetate, maleate, fumarate, citrate, oxalate, succinate, tartrate, malate, mandelate, methane sulphonate, and p-toluenesulphonate. Examples of the alkali addition salts include inorganic salts, such as, for example, sodium, potassium, calcium and ammonium salts, and organic alkali salts, such as, for example, ethylenediamine, ethanolamine, N,N-dialkylenethanolamine, triethanolamine, and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

The term “solvate” in accordance with this invention should be understood as meaning any form of the active compound in accordance with the invention in which the said compound is bonded by a non-covalent bond to another molecule (normally a polar solvent), including especially hydrates and alcoholates.

The term “pharmacological composition,” “therapeutic composition,” “therapeutic formulation” or “pharmaceutically acceptable formulation” can mean, but is in no way limited to, a composition or formulation that allows for the effective distribution of an agent provided by the invention, which is in a form suitable for administration to the physical location most suitable for their desired activity, e.g., systemic administration. The pharmaceutical composition facilitates administration of the compound to an organism. Multiple techniques of administering a compound exist in the art including, but not limited to, intravenous, oral, aerosol, parenteral, ophthalmic, pulmonary and topical administration.

The term “therapeutic” as used herein means a treatment and/or prophylaxis. A therapeutic effect is obtained by suppression, diminution, remission, or eradication of at least one sign or symptom of a disease or disorder state.

As used herein, the terms “therapeutic compound”, “therapeutic agent”, “drug”, “active pharmaceutical”, and “active pharmaceutical ingredient” are used interchangeably to refer to chemical entities that display certain pharmacological effects in a body and are administered for such purpose. Non-limiting examples of therapeutic agents include, but are not limited to, hydrophilic therapeutic agents, hydrophobic therapeutic agents, antibiotics, antibodies, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more therapeutic agents are water-soluble, poorly water-soluble drug or a drug with a low, medium, or high melting point. The therapeutic agents may be provided with or without a stabilizing salt or salts.

Some examples of active ingredients suitable for use in the pharmaceutical formulations and methods of the present invention include: hydrophilic, lipophilic, amphiphilic or hydrophobic, and that can be solubilized, dispersed, or partially solubilized and dispersed, on or about the nanocluster. The active agent-nanocluster combination may be coated further to encapsulate the agent-nanocluster combination and may be directed to a target by functionalizing the nanocluster with, e.g., aptamers and/or antibodies. Alternatively, an active ingredient may also be provided separately from the solid pharmaceutical composition, such as for co-administration. Such active ingredients can be any compound or mixture of compounds having therapeutic or other value when administered to an animal, particularly to a mammal, such as drugs, nutrients, cosmeceuticals, nutraceuticals, diagnostic agents, nutritional agents, and the like. The active agents described herein may be found in their native state, however, they will generally be provided in the form of a salt. The active agents described herein include their isomers, analogs, and derivatives.

An “effective amount” or “therapeutically effective amount”, as used herein, means an amount, which provides a therapeutic or prophylactic benefit. An “effective amount” or “therapeutically effective amount” of a compound is that amount of compound, which is sufficient to provide a beneficial effect to the subject to which the compound is administered. For example, a “therapeutically effective amount” of the nanoparticles and/or the conjugates is the amount that is sufficient or effective to prevent or treat (delay or prevent the onset of, prevent the progression of, inhibit, decrease or reverse) a disease or condition, including alleviating symptoms of such diseases.

A “disease” is a state of health of a subject wherein the subject cannot maintain homeostasis, and wherein if the disease is not ameliorated then the subject's health continues to deteriorate.

In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

To “treat” a disease as the term is used herein, means to reduce the frequency or severity of at least one sign or symptom of a disease or disorder experienced by a subject.

As used herein, “treating a disease or disorder” means reducing the frequency with which a symptom of the disease or disorder is experienced by a patient. Disease and disorder are used interchangeably herein.

A disease or disorder is “alleviated” if the severity of a sign or symptom of the disease or disorder, the frequency with which such a sign or symptom is experienced by a patient, or both, is reduced.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject.

As used herein, the term “diagnosis” refers to the determination of the presence of a disease or disorder. In some embodiments of the present invention, methods for making a diagnosis are provided which permit determination of the presence of a particular disease or disorder.

The term “abnormal” when used in the context of organisms, tissues, cells, or components thereof, refers to those organisms, tissues, cells, or components thereof that differ in at least one observable or detectable characteristic (e.g., age, treatment, time of day, etc.) from those organisms, tissues, cells, or components thereof that display the “normal” (expected) respective characteristic. Characteristics which are normal or expected for one cell or tissue type, might be abnormal for a different cell or tissue type.

“Parenteral” administration of a composition includes, e.g., subcutaneous (s.c.), intravenous (i.v.), intramuscular (i.m.), or intrasternal injection, or infusion techniques.

As used herein, the terms “targeting domain”, “targeting moiety”, or “targeting group” are used interchangeably and refer to all molecules capable of specifically binding to a particular target molecule and forming a bound complex as described above. Thus, the ligand and its corresponding target molecule form a specific binding pair.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody, which is generated using recombinant DNA technology, such as, for example, an antibody expressed by a bacteriophage. The term should also be construed to mean an antibody, which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology, which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned) or other technology, which is available and well known in the art.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequence or a partial nucleotide sequence encoding a protein that elicits an adaptive immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “adjuvant” as used herein is defined as any molecule to enhance an antigen-specific adaptive immune response.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

In certain instances, the polynucleotide or nucleic acid of the invention is a “nucleoside-modified nucleic acid,” which refers to a nucleic acid comprising at least one modified nucleoside. A “modified nucleoside” refers to a nucleoside with a modification.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “amino acid”, “amino acidic monomer”, or “amino acid residue” refer to any of the twenty naturally occurring amino acids including synthetic amino acids with unnatural side chains and including both D and L optical isomers.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A “fragment” of a peptide sequence or a nucleic acid sequence that encodes an antigen may be 100% identical to the full length except missing at least one amino acid or at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more, or 99% or more identical to the antigen and additionally optionally comprise sequence encoding an N terminal methionine or heterologous signal peptide, which is not included when calculating percent identity.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In one aspect, the present invention provides a nanoparticle that selectively targets at least one cell comprising MFSD2A (e.g., endothelial cells of the BBB/BRB). In another aspect, the present invention provides a conjugate that selectively targets at least one cell comprising MFSD2A (e.g., endothelial cells of the BBB). In some embodiments, the nanoparticle and/or the conjugate of the invention crosses the BBB and/or BRB. In some embodiments, the nanoparticle and/or the conjugate comprises a diagnostic agent, imaging agent, therapeutic agent, or any combination thereof.

In one aspect, the present invention relates to a method for in vivo delivery of a diagnostic agent, imaging agent, therapeutic agent, or any combination thereof to a brain using at least one nanoparticle and/or at least one conjugate of the invention. In one aspect, the present invention relates to a method of preventing or treating a neurological or cognitive disease or disorder or a brain disease or disorder (e.g. brain tumor, neurodegenerative disease, food intake disorder, schizophrenia, depression, addiction, etc.) using at least one nanoparticle and/or at least one conjugate of the invention. In one aspect, the present invention relates to a method of regulating a condition associated with the brain (e.g., opioids overdose, temperature regulation, mood, etc.) using at least one nanoparticle and/or at least one conjugate of the invention.

### Conjugates and Nanoparticles

The present invention relates, in part, to a nanoparticle comprising at least one ligand that selectively targets MFSD2A. In various embodiments, the ligand is encapsulated within the nanoparticle, adhered to the surface of the nanoparticle, integrated into the structure of the nanoparticle, bound to the nanoparticle, or any combination thereof. Examples of such ligands include, but are not limited to, fatty acid-lysophosphatidylcholine (LysoPC) (e.g., C2-C28 fatty acid-LysoPC), such as docosahexaenoic acid LysoPC (DHA-LysoPC), eicosapentaenoic acid LysoPC (EPA-LysoPC), linoleic acid LysoPC (LA-LysoPC), or linolelaidic acid LysoPC, acyl-carnitine (e.g., C2-C28 acyl-carnitine), such as tetradecenoyl-carnitine, palmitoyl-carnitine, or 3-hydroxypalmitoyl-carnitine, EPA-carnitine, DHA-carnitine, lysophosphatidic acid (LysoPA), lysophosphatidylethanolamine (LysoPE), lysophosphatidylinositol (LysoPI), lysophosphatidylserine (LysoPS), or any combination thereof.

In various embodiments, the nanoparticle comprises the ligand in a concentration range of about 0.01 mol % to about 99.99 mol %. In some embodiments, the nanoparticle comprises the ligand in a concentration range of about 0.1 mol % to about 99.9 mol %. In some embodiments, the nanoparticle comprises the ligand in a concentration range of about 1 mol % to about 70 mol %. In some embodiments, the nanoparticle comprises the ligand in a concentration range of about 1 mol % to about 60 mol %. In some embodiments, the nanoparticle comprises the ligand in a concentration range of about 1 mol % to about 55 mol %. In some embodiments, the nanoparticle comprises the ligand in a concentration range of about 1 mol % to about 50 mol %. In some embodiments, the nanoparticle comprises the ligand in a concentration range of about 10 mol % to about 50 mol %.

For example, in some embodiments, the nanoparticle comprises the ligand in a concentration of about 0.01 mol %. In some embodiments, the nanoparticle comprises the ligand in a concentration of about 0.02 mol %. In some embodiments, the nanoparticle comprises the ligand in a concentration of about 0.05 mol %. In some embodiments, the nanoparticle comprises the ligand in a concentration of about 0.15 mol %. In some embodiments, the nanoparticle comprises the ligand in a concentration of about 1.0 mol %. In some embodiments, the nanoparticle comprises the ligand in a concentration of about 10 mol %.

In various embodiments, the nanoparticle selectively targets MFSD2A. In some embodiments, the nanoparticle selectively binds MFSD2A. In some embodiments, the nanoparticle inhibits or reduces the activity of MFSD2A. In various embodiments, the nanoparticles described herein are formulated for stability for in vivo cell targeting. In some embodiments, the nanoparticle formulated for stability for in vivo delivery to a cell of interest (e.g., a cell comprising a MFSD2A or a fragment thereof, endothelial cell, etc.).

In some embodiments, the nanoparticle selectively targets at least one cell comprising MFSD2A. In one embodiment, the at least one cell is a neuron. In one embodiment, the at least one cell is an endothelial cell. In one embodiment, the endothelial cell is an endothelial cell of a blood brain barrier. In one embodiment, the endothelial cell is a brain-microvascular endothelial cell (BMEC). In one embodiment, the endothelial cell is in blood retinal barrier.

In some embodiments, the MFSD2A receptor is involved in the uptake of nanoparticles. In some embodiments, the MFSD2A receptor is inhibited so that nanoparticle transcytosis is enhanced.

In various embodiments, the nanoparticle has an average hydrodynamic diameter of from about 10 nm to about 10,000 nm, from about 130 nm to about 2,500 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 200 nm, 300 nm, 400 nm, 500 nm, 600 nm, 700 nm, 800 nm, 900 nm, 1,000 nm, 2,000 nm, 2,500 nm, 5,000 nm, or 10,000 nm.

For example, in various embodiments, the nanoparticle has an average particle size (e.g., average hydrodynamic diameter of the nanoparticle) below about 2,500 nm. In some embodiments, the nanoparticle has a particle size (e.g., average hydrodynamic diameter of the nanoparticle) of between about 1 nm to about 2,500 nm. In some embodiments, the nanoparticle has a particle size (e.g., average hydrodynamic diameter of the nanoparticle) of between about 1 nm to about 300 nm. In some embodiments, the nanoparticle has a particle size (e.g., average hydrodynamic diameter of the nanoparticle) of between about 1 nm to about 200 nm.

In some embodiments, the nanoparticle is any type of nanoparticle, including, but not limited to, liposomes, lipid nanoparticles, organic nanoparticles, inorganic nanoparticles (e.g., metal nanoparticles, such as gold nanoparticles, iron nanoparticles, ZnO nanoparticles, TiO₂ nanoparticles, etc.), biocompatible nanoparticles, such as biocompatible organic nanoparticles, biocompatible inorganic nanoparticles, etc., polymer nanoparticles, nanoclusters, nanocapsules, core-shell nanocapsules, nanovesicles, micelles, block copolymer micelles, lamaellae shaped particles, polymersomes, dendrimers, emulsions, exosomes, SEDDS, microspheres, micro-structured lipid carriers, nano-structured lipid carriers, and other nano-size particles of various other small fabrications that are known to those of skill in the art.

In various embodiments, the nanoparticle comprises at least one agent (e.g., a MIFSD2A inhibitor, therapeutic agent, diagnostic agent, etc.). In some embodiments, the nanoparticle encapsulates at least one agent. Thus, in one embodiment, the nanoparticle is a nanocapsule. In one embodiment, the nanoparticle is a nanocarrier.

In one embodiment, the nanoparticle is a liposome. In some embodiments, the liposome comprises phospholipids, cholesterol, sphingolipids, ceramides, hapten-conjugated lipids, or any combination thereof.

In one embodiment, the nanoparticle is a lipid nanoparticle. In some embodiments, the lipid nanoparticles comprise phospholipids, cholesterol, PEG-phospholipids, lipid-soluble vitamins, lipid conjugates, or any combination thereof.

In some embodiments, the nanoparticle comprises one or more lipids. In some embodiments, the lipid is selected from phosphatidylcholine (PC), phosphatidylethanolamine (PE), phosphatidylserine (PS), phosphatidylinositol (PI), phospholipid-phosphatic acid (PA), LysoPC, LysoPE, LysoPS, LysoPI, LysoPA, acyl-carnitine (e.g., C2-C28 acyl-carnitine), or any combination thereof.

In various embodiments, the nanoparticle comprises one or more lipids in a concentration range of about 0.1 mol % to about 100 mol %. In some embodiments, the nanoparticle comprises one or more lipids in a concentration range of about 1 mol % to about 100 mol %. In some embodiments, the nanoparticle comprises one or more lipids in a concentration range of about 1 mol % to about 70 mol %. In some embodiments, the nanoparticle comprises one or more lipids in a concentration range of about 10 mol % to about 80 mol %. In some embodiments, the nanoparticle comprises one or more lipids in a concentration range of about 10 mol % to about 70 mol %. In some embodiments, the nanoparticle comprises one or more lipids in a concentration range of about 10 mol % to about 50 mol %. In some embodiments, the nanoparticle comprises one or more lipids in a concentration range of about 15 mol % to about 45 mol %. In some embodiments, the nanoparticle comprises one or more lipids in a concentration range of about 35 mol % to about 40 mol %.

For example, in some embodiments, the nanoparticle comprises one or more lipids in a concentration of about 1 mol %. In some embodiments, the nanoparticle comprises one or more lipids in a concentration of about 2 mol %. In some embodiments, the nanoparticle comprises one or more lipids in a concentration of about 5 mol %. In some embodiments, the nanoparticle comprises one or more lipids in a concentration of about 5.5 mol %. In some embodiments, the nanoparticle comprises one or more lipids in a concentration of about 10 mol %. In some embodiments, the nanoparticle comprises one or more lipids in a concentration of about 12 mol %. In some embodiments, the nanoparticle comprises one or more lipids in a concentration of about 15 mol %. In some embodiments, the nanoparticle comprises one or more lipids in a concentration of about 20 mol %. In some embodiments, the nanoparticle comprises one or more lipids in a concentration of about 25 mol %. In some embodiments, the nanoparticle comprises one or more lipids in a concentration of about 30 mol %. In some embodiments, the nanoparticle comprises one or more lipids in a concentration of about 35 mol %. In some embodiments, the nanoparticle comprises one or more lipids in a concentration of about 37 mol %. In some embodiments, the nanoparticle comprises one or more lipids in a concentration of about 40 mol %. In some embodiments, the nanoparticle comprises one or more lipids in a concentration of about 45 mol %. In some embodiments, the nanoparticle comprises one or more lipids in a concentration of about 50 mol %. In some embodiments, the nanoparticle comprises one or more lipids in a concentration of about 60 mol %. In some embodiments, the nanoparticle comprises one or more lipids in a concentration of about 70 mol %. In some embodiments, the nanoparticle comprises one or more lipids in a concentration of about 80 mol %. In some embodiments, the nanoparticle comprises one or more lipids in a concentration of about 90 mol %. In some embodiments, the nanoparticle comprises one or more lipids in a concentration of about 95 mol %. In some embodiments, the nanoparticle comprises one or more lipids in a concentration of about 95.5 mol %. In some embodiments, the nanoparticle comprises one or more lipids in a concentration of about 99 mol %. In some embodiments, the nanoparticle comprises one or more lipids in a concentration of about 99.9 mol %. In some embodiments, the nanoparticle comprises one or more lipids in a concentration of about 100 mol %.

In various embodiments, the nanoparticle further comprises at least one helper compound. In some embodiments, the helper compound is a helper lipid, helper polymer, or any combination thereof. In some embodiments, the helper lipid is phospholipid, cholesterol lipid, polymer, cationic lipid, neutral lipid, charged lipid, steroid, steroid analogue, polymer conjugated lipid, stabilizing lipid, or any combination thereof.

In some embodiments, the phospholipid is dioleoyl-phosphatidylethanolamine (DOPE) or a derivative thereof, distearoylphosphatidylcholine (DSPC) or a derivative thereof, distearoyl-phosphatidylethanolamine (DSPE) or a derivative thereof, stearoyloleoylphosphatidylcholine (SOPC) or a derivative thereof, 1-stearioyl-2-oleoyl-phosphatidyethanol amine (SOPE) or a derivative thereof, N-(2,3-dioleoyloxy)propyl)-N,N,N-trimethylammonium chloride (DOTAP) or a derivative thereof, or any combination thereof.

In some embodiments, the cholesterol lipid is cholesterol or a derivative thereof, such as a substituted cholesterol molecule. In some embodiments, the nanoparticle comprises a mixture of cholesterol and a substituted cholesterol molecule.

In some embodiments, the polymer is polyethylene glycol (PEG) or a derivative thereof.

As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In some embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In one embodiment, the cationic lipid is an amino lipid. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In one embodiment, the lipid is a PEGylated lipid, including, but not limited to, DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid.

The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), distearoyl-phosphatidylethanolamine (DSPE)-maleimide-PEG, distearoyl-phosphatidylethanolamine (DSPE)-maleimide-PEG2000, 16-O-monomethyl PE, 16-O-dimethyl PE, 18-1-trans PE, 1-stearioyl-2-oleoyl-phosphatidyethanol amine (SOPE), stearoyloleoylphosphatidylcholine (SOPC), and 1,2-dielaidoyl-sn-glycero-3-phophoethanolamine (transDOPE). In one embodiment, the neutral lipid is 1,2-distearoyl-sn-glycero-3-phosphocholine (DSPC).

In some embodiments, the composition comprises a neutral lipid selected from DSPC, DPPC, DMPC, DOPC, POPC, DOPE, and SM.

A “steroid” is a compound comprising the following carbon skeleton:

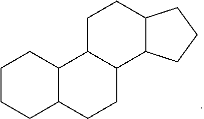 [SMILES: C1CCC2C(C1)CCC1C3CCCC3CCC21]

In certain embodiments, the steroid or steroid analogue is cholesterol. In some of these embodiments, the molar ratio of the cationic lipid.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include polyethylene glycol (PEG), maleimide PEG (mPEG), DSPE-PEG-DBCO, 1-(monomethoxy-polyethyleneglycol)-2,3-dimyristoylglycerol (PEG-s-DMG), DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid and the like.

In certain embodiments, the stabilizing lipid is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)₂₀₀₀)carbamyl]-1,2-dimyristyloxlpropyl-3-amine (PEG-c-DMA). In one embodiment, the polyethylene glycol-lipid is PEG-c-DOMG). In other embodiments, the nanoparticles comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate.

In various embodiments, the nanoparticle comprises one or more helper compound in a concentration range of about 0 mol % to about 100 mol %. In some embodiments, the nanoparticle comprises one or more helper compound in a concentration range of about 0.01 mol % to about 99.99 mol %. In some embodiments, the nanoparticle comprises one or more helper compound in a concentration range of about 0.1 mol % to about 99.9 mol %. In some embodiments, the nanoparticle comprises one or more helper compound in a concentration range of about 0.1 mol % to about 90 mol %. In some embodiments, the nanoparticle comprises one or more helper compound in a concentration range of about 0.1 mol % to about 70 mol %. In some embodiments, the nanoparticle comprises one or more helper compound in a concentration range of about 5 mol % to about 95 mol %. In some embodiments, the nanoparticle comprises one or more helper compound in a concentration range of about 0.5 mol % to about 50 mol %. In some embodiments, the nanoparticle comprises one or more helper compound in a concentration range of about 0.5 mol % to about 47 mol %. In some embodiments, the nanoparticle comprises one or more helper compound in a concentration range of about 2.5 mol % to about 47 mol %.

For example, in some embodiments, the nanoparticle comprises one or more helper compound in a concentration of about 0.01 mol %. In some embodiments, the nanoparticle comprises one or more helper compound in a concentration of about 0.1 mol %. In some embodiments, the nanoparticle comprises one or more helper compound in a concentration of about 0.5 mol %. In some embodiments, the nanoparticle comprises one or more helper compound in a concentration of about 1 mol %. In some embodiments, the nanoparticle comprises one or more helper compound in a concentration of about 1.5 mol %. In some embodiments, the nanoparticle comprises one or more helper compound in a concentration of about 2 mol %. In some embodiments, the nanoparticle comprises one or more helper compound in a concentration of about 2.5 mol %. In some embodiments, the nanoparticle comprises one or more helper compound in a concentration of about 5 mol %. In some embodiments, the nanoparticle comprises one or more helper compound in a concentration of about 10 mol %. In some embodiments, the nanoparticle comprises one or more helper compound in a concentration of about 12 mol %. In some embodiments, the nanoparticle comprises one or more helper compound in a concentration of about 15 mol %. In some embodiments, the nanoparticle comprises one or more helper compound in a concentration of about 16 mol %. In some embodiments, the nanoparticle comprises one or more helper compound in a concentration of about 20 mol %. In some embodiments, the nanoparticle comprises one or more helper compound in a concentration of about 25 mol %. In some embodiments, the nanoparticle comprises one or more helper compound in a concentration of about 30 mol %. In some embodiments, the nanoparticle comprises one or more helper compound in a concentration of about 35 mol %. In some embodiments, the nanoparticle comprises one or more helper compound in a concentration of about 37 mol %. In some embodiments, the nanoparticle comprises one or more helper compound in a concentration of about 40 mol %. In some embodiments, the nanoparticle comprises one or more helper compound in a concentration of about 45 mol %. In some embodiments, the nanoparticle comprises one or more helper compound in a concentration of about 46.5 mol %. In some embodiments, the nanoparticle comprises one or more helper compound in a concentration of about 47 mol %. In some embodiments, the nanoparticle comprises one or more helper compound in a concentration of about 50 mol %. In some embodiments, the nanoparticle comprises one or more helper compound in a concentration of about 60 mol %. In some embodiments, the nanoparticle comprises one or more helper compound in a concentration of about 63 mol %. In some embodiments, the nanoparticle comprises one or more helper compound in a concentration of about 70 mol %. In some embodiments, the nanoparticle comprises one or more helper compound in a concentration of about 80 mol %. In some embodiments, the nanoparticle comprises one or more helper compound in a concentration of about 90 mol %. In some embodiments, the nanoparticle comprises one or more helper compound in a concentration of about 95 mol %. In some embodiments, the nanoparticle comprises one or more helper compound in a concentration of about 95.5 mol %. In some embodiments, the nanoparticle comprises one or more helper compound in a concentration of about 99 mol %. In some embodiments, the nanoparticle comprises one or more helper compound in a concentration of about 100 mol %.

In various aspects, the nanoparticle comprises one or more stabilizers. In one embodiment, the stabilizer comprises a biocompatible polymer. Examples of stabilizers include, but are not limited to, biocompatible polymer, a biodegradable polymer, a multifunctional linker, starch, modified starch, and starch derivatives, gums, including but not limited to polymers, polypeptides, albumin, amino acids, alcohols (e.g., PVA, ethyl alcohol, etc.), thiols, amines, carboxylic acid and combinations or derivatives thereof, citric acid, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum, polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone), monomeric, dimeric, oligomeric or long-chain, copolymers, block polymers, block co-polymers, polymers, PEG, dextran, modified dextran, polyvinylalcohol, polyvinylpyrollidone, polyacrylates, polymethacrylates, polyanhydrides, polypeptides, albumin, alginates, amino acids, thiols, amines and carboxylic acids or combinations thereof.

In some embodiments, the nanoparticle comprises one or more molecules selected from the group consisting of phospholipids, albumin, dextran, gelatin, poly(ethylene glycerol) (PEG), poly(vinylpyrrolidone), hyaluronic acid, heparin, heparin sulfate, sialic acid, poly(N-acetylglucosamine) (Chitin), Chitosan, poly(3-hydroxyvalerate), poly(D,L-lactide-co-glycolide), poly(1-lactide-co-glycolide), poly(3-hydroxybutyrate), poly(4-hydroxybutyrate), poly(3-hydroxybutyrate-co-3-hydroxyvalerate), polyorthoester, polyanhydride, poly(glycolic acid), poly(glycolide), poly(L-lactic acid), poly(L-lactide), poly(D,L-lactic acid), poly(D,L-lactide), poly(L-lactide-co-D,L-lactide), poly(caprolactone), poly(L-lactide-co-caprolactone), poly(D,L-lactide-co-caprolactone), poly(glycolide-co-caprolactone), poly(trimethylene carbonate), polyester amide, poly(glycolic acid-co-trimethylene carbonate), co-poly(ether-esters) (e.g. PEO/PLA), polyphosphazenes, fibrin, fibrin glue, fibrinogen, cellulose, starch, collagen and hyaluronic acid, elastin and hyaluronic acid, polyurethanes, silicones, polyesters, polyolefins, polyisobutylene and ethylene-alphaolefin copolymers, acrylic polymers and copolymers other than polyacrylates, vinyl halide polymers and copolymers, polyvinyl chloride, polyvinyl ethers, polyvinyl methyl ether, polyvinylidene halides, polyvinylidene chloride, poly(vinylidene fluoride), poly(vinylidene fluoride-co-hexafluoropropylene), polyacrylonitrile, polyvinyl ketones, polyvinyl aromatics, polystyrene, polyvinyl esters, polyvinyl acetate, acrylonitrile-styrene copolymers, ABS resins, polyamides, Nylon 66, polycaprolactam, polycarbonates including tyrosine-based polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose, fullerenes, lipids, and any combination thereof.

In some embodiments, the nanoparticle comprises one or more molecules selected from the group consisting of gelatin, albumin, dextrose, dextran, a high molecular weight poly(ethylene glycol) or a high molecular weight poly(vinylpyrrolidone), hyaluronic acid, heparin, heparin sulfate, sialic acid, Chitosan, and any combination thereof.

In one embodiment, the nanoparticle is a polymersome. Any polymersome known in the art may be utilized. Thus, in one embodiment, the nanoparticle comprises a homopolymer. In some embodiments, the nanoparticle comprises a block copolymer that is a triblock, tetrablock, pentablock, or at least six block copolymer. In some embodiments, the nanoparticle comprises poly(ethylene oxide) (PEO) block copolymer, poly(ethylethylene) (PEE), poly(butadiene) (PB or PBD), poly(styrene) (PS), poly(isoprene) (PI), PEI, poly(lactide-co-glycolic acid) (PLGA), biodegradable PLGA, polyethylene glycol (PEG), poly(lactide-co-glycolic acid)-polyethylene glycol (PLGA-PEG), poly(lactide-co-glycolic acid)-block-polyethylene glycol (PLGA-b-PEG), biodegradable PLGA-PEG, biodegradable PLGA-b-PEG, polyanhydride, polyanhydride-block-PEG copolymers, zwitterionic poly(carbobetaine), zwitterionic poly(sulfobetaine)-containing, zwitterionic poly(carbobetaine) and zwitterionic poly(sulfobetaine)-containing copolymers, poly(acrylic acid-co-distearin acrylate), poly(trimethylene carbonate)-block-poly(L-glutamic acid), poly(ethylene glycol-block-L-aspartic acid), poly(2-hydroxyethyl-co-octadecyl aspartamide), poly(ethylene glycol-co-trimethylene carbonate-co-caprolactone, polypropylene oxide block copolymers, polyethylene oxide-block-polypropylene oxide copolymers, or any combination thereof.

In some embodiments, the nanoparticle comprises poly(F-caprolactone) (PCL) diblock co-polymer. In some embodiments, the nanoparticle comprises poly(ethylene oxide)-block-poly(F-caprolactone) (PEO-b-PCL) based diblock copolymers. In some embodiments, the nanoparticle is derived from the coupling of poly(lactic acid), poly(glycolide), poly(lactic-coglycolic acid) and/or or poly(3-hydroxybutyrate) with PEO. In some embodiments, the nanoparticle comprises PLGA. In some embodiments, the nanoparticle comprises PEG. In one embodiment, the nanoparticle comprises poly(lactide-co-glycolic acid)-polyethylene glycol (PLGA-PEG). For example, in some embodiments, a PLGA-PEG polymersome encapsulates the ICGJ and optionally PEI.

In one embodiment, the nanoparticle further comprises a cationic polymer. The cationic polymer may be a straight chain polymer (i.e., linear polymer) or a branched chain polymer (i.e., branched polymer), including hyperbranched polymers. In one embodiment the cationic polymer is a branched cationic polymer. In one embodiment, the cationic polymer is cross-linked. In one embodiment, the cationic polymer is a polyamine. In one embodiment, the cationic polymer has molecular weight of 5 kDa-3000 kDa. For example, in one embodiment, the cationic polymer has a molecular weight of 5 kDa-2000 kDa, 5 kDa-1500 kDa, 5 kDa-1000 kDa, 5 kDa-800 kDa, 5 kDa-500 kDa, 5 kDa-300 kDa or 5 kDa-200 kDa or 800 kDa-3000 kDa.

In one embodiment, the cationic polymer is a polyalkyleneimine (e.g., polyethyleneimine), polyallylamine, polyamidoamine, or poly(amino-co-ester). In one embodiment, the cationic polymer is polyethyleneimine (PEI), chitosan, poly(2-N,N-dimethylaminoethylmethacrylate), or poly-L-lysine. In one embodiment, the cationic polymer stabilizes the nanocapsule.

In some embodiments, the nanoparticles comprise a biodegradable polymer comprises PLGA, poly(D,L-lactide-co-glycolide), poly(D,L-lactide), poly(D,L-lactide-co-lactide), poly(L-lactide), poly(glycolide), poly(L-lactide-co-glycolide), poly(caprolactone), poly(glycolide-co-trimethylene carbonate), poly(3-hydroxybutyrate), poly(3-hydroxybutyrate-co-3-hydroxyvalerate), poly(4-hydroxybutyrate), poly(ester amide), poly(ester-sulfoester amide), poly(orthoester) or poly(anhydride), and a combination thereof.

In various embodiments, the nanoparticle of the present invention is substantially non-toxic.

In one embodiment, the nanoparticle is a biodegradable nanoparticle. In one embodiment, the nanoparticle is biodegradable nanocapsule. In one embodiment, the nanoparticle is a biodegradable polymer vesicle. In one embodiment, the nanoparticle is a biodegradable liposome.

In various embodiments, the nanoparticle comprises at least one agent.

The present invention also relates, in part, to a conjugate comprising at least one ligand that selectively targets MFSD2A (e.g., fatty acid-LysoPC, DHA-LysoPC, EPA-LysoPC, LA-LysoPC, or linolelaidic acid LysoPC, acyl-carnitine, tetradecenoyl-carnitine, palmitoyl-carnitine, 3-hydroxypalmitoyl-carnitine, EPA-carnitine, DHA-carnitine, LysoPA, LysoPE, LysoPI, LysoPS, etc.). In various embodiments, the conjugate comprises a covalent and/or non-covalent linkage of the ligand that selectively targets MFSD2A to at least one agent. Thus, in various embodiments, the conjugate selectively targets MFSD2A. In some embodiments, the conjugate selectively binds MFSD2A. In some embodiments, the conjugate inhibits or reduces the activity of MFSD2A. In various embodiments, the conjugates described herein are formulated for stability for in vivo cell targeting. In some embodiments, the conjugate formulated for stability for in vivo delivery to a cell of interest (e.g., a cell comprising a MFSD2A or a fragment thereof, endothelial cell, etc.). In some embodiments, the conjugate selectively targets at least one cell comprising MFSD2A.

In one embodiment, the ligand that selectively targets MFSD2A is bound directly to the at least one agent. In one embodiment, the ligand that selectively targets MFSD2A is bound directly to the surface of at least one agent. In one embodiment, the ligand that selectively targets MFSD2A is bound to the at least one agent using a linking molecule. In one embodiment, the ligand that selectively targets MFSD2A is bound to the surface of the at least one agent using a linking molecule.

The linking molecules useful in the conjugates of the present invention may be any molecule capable of binding to both the ligand used in the conjugates of the present invention and the at least one agent used in the conjugates of the present invention. In certain embodiments, the linking molecule may be a hydrophilic polymer. Examples of linking molecules include, but are not limited to, poly(ethylene glycol) and its derivatives, azide compounds, maleimide compounds, hydrazine compounds, dibenzo-cyclooctyne (DBCO) compounds, dithiol compounds, dithiol compounds with hydrazide and/or carboxylic functionality, or single thiols and/or amines, or their derivatives.

In certain embodiments, the linking molecule and the ligand may be bound by one or more covalent bonds. In certain embodiments, the linking molecule, in addition to linking the ligand that selectively targets MFSD2A and the at least one agent, may impart certain benefits upon the conjugates of the present invention, including, but not limited to, improved hydrophilicity and stability in solution, reduced immunogenic responses upon introduction of the conjugates of the present invention into a subject, increased circulation time of the conjugates of the present invention when introduced into the bloodstream of a subject. The choice of a linking molecule may depend upon, among other things, the ligand chosen and the subject into which the conjugates of the present invention are to be introduced. One of ordinary skill in the art, with the benefit of this invention, will recognize additional suitable linking molecules. Such linking molecules are considered to be within the spirit of the present invention.

In some embodiments, the agent is any agent described herein, such as any drug, lipid, polymer, peptide, polypeptide, and/or polypeptide variant described herein. In various embodiments, the conjugate of the present invention is substantially non-toxic. In one embodiment, the conjugate is a biodegradable conjugate.

In some embodiments, the agent is adhered to the surface of the nanoparticle and/or the conjugate of the invention. In some embodiments, the agent is integrated into the structure of the nanoparticle and/or the conjugate of the invention. In some embodiments, the nanoparticle and/or the conjugate of the invention encapsulates at least one agent. In some embodiments, the nanoparticle and/or the conjugate of the invention comprises at least two agents. For example, in some embodiments, the nanoparticle comprises a first agent that provides an indicium for the presence of blood brain barrier or blood retina barrier site and a second agent that exhibits a therapeutic effect on the blood brain barrier or blood retina barrier site. In some embodiments, the indicium is a fluorescent signal emitted upon binding of the first agent to or near the blood brain barrier or blood retina barrier site.

In one aspect, the invention is not limited to any particular cargo or agent for which the nanoparticle and/or the conjugate of the invention is able to carry or transport. Rather, the invention includes any agent that can be carried by the nanoparticle and/or the conjugate of the invention. For example, the agents that can be carried by the nanoparticle and/or the conjugate of the invention include, but are not limited to, diagnostic agents, imaging agents, detectable agents (e.g., dyes), and therapeutic agents.

In one embodiment, the agent is a MFSD2A inhibitor. In one embodiment, the nanoparticle and/or the conjugate of the invention encapsulates the MFSD2A inhibitor. In one embodiment, the nanoparticle and/or the conjugate of the invention is bound to the MFSD2A inhibitor. Examples of MFSD2A inhibitors include, but are not limited to, Tunicamycin.

In one aspect of the invention, the agent is a therapeutic agent. In one embodiment, the nanoparticle and/or the conjugate of the invention encapsulates the therapeutic agent. In one embodiment, the nanoparticle and/or the conjugate of the invention is bound to the therapeutic agent. In one embodiment, the therapeutic agent is a hydrophobic therapeutic agent. In one embodiment, the therapeutic agent is a hydrophilic therapeutic agent. Examples of such therapeutic agents include, but are not limited to, one or more drugs, proteins, amino acids, peptides, antibodies, antibiotics, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), medical imaging agents, therapeutic moieties, one or more non-therapeutic moieties or a combination to target cancer or atherosclerosis, selected from folic acid, peptides, proteins, aptamers, antibodies, siRNA, poorly water soluble drugs, anti-cancer drugs, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents, or any combinations thereof.

In one embodiment, the therapeutic agent is one or more non-therapeutic moieties. In some embodiments, the nanoparticle and/or the conjugate of the invention comprises one or more therapeutic moieties, one or more non-therapeutic moieties, or any combination thereof.

In one embodiment, the therapeutic moiety targets cancer. In some embodiments, the composition comprises folic acid, peptides, proteins, aptamers, antibodies, small RNA molecules, miRNA, shRNA, siRNA, poorly water-soluble therapeutic agents, anti-cancer agents, or any combinations thereof.

In one embodiment, the therapeutic agent may be an anti-cancer agent. Any suitable anti-cancer agent may be used in the compositions and methods of the present disclosure. The selection of a suitable anti-cancer agent may depend upon, among other things, the type of cancer to be treated and the compositions of the present disclosure. In certain embodiments, the anti-cancer agent may be effective for treating one or more of pancreatic cancer, esophageal cancer, rectal cancer, colon cancer, prostate cancer, kidney cancer, liver cancer, breast cancer, ovarian cancer, and stomach cancer. Examples of anti-cancer agents include, but is not limited to, chemotherapeutic agents, antiproliferative agents, anti-tumor agents, checkpoint inhibitors, and anti-angiogenic agents. For example, in one embodiment, the anti-cancer agent is gemcitabine, doxorubicin, 5-Fu, tyrosine kinase inhibitors, sorafenib, trametinib, rapamycin, fulvestrant, ezalutamide, or paclitaxel.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The agent can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

In some embodiments, the anti-cancer agent may be a prodrug form of an anti-cancer agent. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. The use of a prodrug form of an anti-cancer agent in the compositions, among other things, may increase the concentration of the anti-cancer agent in the compositions of the present disclosure. In certain embodiments, an anti-cancer agent may be chemically modified with an alkyl or acyl group or some form of lipid. The selection of such a chemical modification, including the substituent(s) to add and/or remove to create the prodrug, may depend upon a number of factors including, but not limited to, the particular drug and the desired properties of the prodrug. One of ordinary skill in the art, with the benefit of this disclosure, will recognize suitable chemical modifications.

In some embodiments, the agent comprises one or more neurodegenerative disease drugs. Exemplary neurodegenerative disease drugs include, but are not limited to, Amantadine (SYMMETREL), Apomorphine (APOKYN), Baclofen (LIORESAL), Carbidopa (LODOSYN), Carbidopa/levodopa (SINEMET, ATAMET, others; orally disintegrating tablet, PARCOPA), Dantrolene (DANTRIUM), Donepezil (ARICEPT), Entacapone (COMTAN; fixed combination with carbidopa/levodopa, STAVELO), Galantamine (NIVALIN, others), Levodopa (L-DOPA, LARODOPA), Memantine (NAMENDA), Pramipexole (MIRAPEX), Rasagiline (AZILECT), Riluzole (RILUTEK), Rivastigmine (EXELON), Ropinirole (REQUIP), Selegiline (ELDEPRYL; oral disintegrating tablet, EMSAM; transdermal patch, ZELAPAR), Tacrine (COGNEX), Tetrabenazine (XENAZINENITOMAN), Tizanidine (ZANAFLEX), Tolcapone (TASMAR).

In some embodiments, the agent comprises one or more drug to treat depression.

Exemplary drugs that may be used to treat depression include, but are not limited to, Abilify (aripiprazole), Adapin (doxepin), Anafranil (clomipramine), Aplenzin (bupropion), Asendin (amoxapine), Aventyl HCl (nortriptyline), Brexipipzole (Rexulti), Celexa (citalopram), Cymbalta (duloxetine), Desyrel (trazodone), Effexor XR (venlafaxine), Emsam (selegiline), Esketamine (Spravato), Etrafon (perphenazine and amitriptyline), Elavil (amitriptyline), Endep (amitriptyline), Fetzima (levomilnacipran), Khedezla (desvenlafaxine), Latuda (lurasidone), Lamictal (lamotrigine), Lexapro (escitalopram), Limbitrol (amitriptyline and chlordiazepoxide), Marplan (isocarboxazid), Nardil (phenelzine), Norpramin (desipramine), Oleptro (trazodone), Pamelor (nortriptyline), Parnate (tranylcypromine), Paxil (paroxetine), Pexeva (paroxetine), Prozac (fluoxetine), Pristiq (desvenlafaxine), Remeron (mirtazapine), Sarafem (fluoxetine), Seroquel XR (quetiapine), Serzone (nefazodone), Sinequan (doxepin), Surmontil (trimipramine), Symbyax (fluoxetine and the atypical antipsychotic drug olanzapine), Tofranil (imipramine), Triavil (perphenazine and amitriptyline), Trintelllix (vortioxetine), Viibryd (vilazodone), Vivactil (protriptyline), Wellbutrin (bupropion), Zoloft (sertraline), and Zyprexa (olanzapine).

In some embodiments, the agent comprises one or more drug to treat schizophrenia. Exemplary drugs that may be used to treat schizophrenia include, but are not limited to, Chlorpromazine (Thorazine), Fluphenazine (Prolixin), Haloperidol (Haldol), Perphenazine (Trilafon), Thioridazine (Mellaril), Thiothixene (Navane), Trifluoperazine (Stelazine) Aripiprazole (Abilify), Aripiprazole lauroxil (Aristada), Asenapine (Saphris), Brexpiprazole (Rexulti), Cariprazine (Vraylar), Clozapine (Clozaril), Iloperidone (Fanapt), Lumateperonee (Caplyta), Lurasidone (Latuda), Olanzapine (Zyprexa), Olanzapine/samidorphan (Lybalvi), Paliperidone (Invega Sustenna), Paliperidone palmitate (Invega Trinza), Quetiapine (Seroquel), Risperidone (Risperdal), and Ziprasidone (Geodon).

In some embodiments, the agent comprises one or more drug to treat a food intake disorder, such as obesity. Exemplary drugs that may be used to treat food intake disorder, such as obesity include, but are not limited to, Phentermine, Lorcaserin, Phentermine/topiramate extended-release (ER), Naltrexone short-release (SR)/bupropion SR, or any combination thereof.

In some embodiments, the agent comprises one or more drug to treat addiction. Exemplary drugs that may be used to treat addiction include, but are not limited to, Buprenorphine, Methadone, Naltrexone, Acamprosate (Campral®), Disulfiram (Antabuse®), Bupropion, Nicotine Replacement Therapies (NRTs), and Varenicline.

In some embodiments, the agent comprises one or more drug to regulate body temperature.

In some embodiments, the agent comprises one or more gene components, such as siRNA or therapeutic DNA fragments. In some embodiments, the gene component is encapsulated in the nanoparticle. In some embodiments, the gene component is on the surface of the nanoparticle, for example, attached to or within the coating material.

In some embodiments, the nanoparticle further comprises a biocompatible metal. Examples of biocompatible metals include, but are not limited to, copper, copper sulfide, iron oxide, cobalt and noble metals, such as gold and/or silver. One of ordinary skill in the art will be able to select of a suitable type of nanoparticle taking into consideration at least the type of imaging and/or therapy to be performed.

One of skill in the art would understand that different embodiments of conjugates disclosed herein and different embodiments of nanoparticles disclosed herein can be used in connection with any aspect of the described invention.

### Small Molecule

In various embodiments, the agent is a small molecule. In various embodiments, the agent is a therapeutic agent. In various embodiments, the therapeutic agent is a small molecule. When the therapeutic agent is a small molecule, a small molecule may be obtained using standard methods known to the skilled artisan. Such methods include chemical organic synthesis or biological means. Biological means include purification from a biological source, recombinant synthesis, and in vitro translation systems, using methods well known in the art. In one embodiment, a small molecule therapeutic agent comprises an organic molecule, inorganic molecule, biomolecule, synthetic molecule, and the like.

Combinatorial libraries of molecularly diverse chemical compounds potentially useful in treating a variety of diseases and conditions are well known in the art, as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development. In some embodiments of the invention, the therapeutic agent is synthesized and/or identified using combinatorial techniques.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores. In some embodiments of the invention, the therapeutic agent is synthesized via small library synthesis.

The small molecule and small molecule compounds described herein may be present as salts even if salts are not depicted, and it is understood that the invention embraces all salts and solvates of the therapeutic agents depicted here, as well as the non-salt and non-solvate form of the therapeutic agents, as is well understood by the skilled artisan. In some embodiments, the salts of the therapeutic agents of the invention are pharmaceutically acceptable salts.

Where tautomeric forms may be present for any of the therapeutic agents described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diastereomeric forms of the therapeutic agents described. The recitation of the structure or name herein is intended to embrace all possible stereoisomers of therapeutic agents depicted. All forms of the therapeutic agents are also embraced by the invention, such as crystalline or non-crystalline forms of the therapeutic agent. Compositions comprising therapeutic agents of the invention are also intended, such as a composition of substantially pure therapeutic agent, including a specific stereochemical form thereof, or a composition comprising mixtures of therapeutic agents of the invention in any ratio, including two or more stereochemical forms, such as in a racemic or non-racemic mixture.

The invention also includes any or all active analog or derivative, such as a prodrug, of any therapeutic agent described herein. In one embodiment, the therapeutic agent is a prodrug. In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule therapeutic agents described herein are derivatives or analogs of known therapeutic agents, as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be carbocyclic or heterocyclic.

In one embodiment, the small molecule therapeutic agents described herein can independently be derivatized, or analogs prepared therefrom, by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Nucleic Acid Molecule

In other related aspects, the agent is a nucleic acid molecule. In various embodiments, the agent is an isolated nucleic acid. Thus, in one embodiment, an isolated nucleic acid, including for example a DNA oligonucleotide and an RNA oligonucleotide can be incorporated in the composition of the invention. In other related aspects, the therapeutic agent is an isolated nucleic acid. In certain embodiments, the isolated nucleic acid molecule is one of a DNA molecule or an RNA molecule. In certain embodiments, the isolated nucleic acid molecule is a cDNA, mRNA, siRNA, shRNA or miRNA molecule. In one embodiment, the isolated nucleic acid molecule encodes a therapeutic peptide. In some embodiments, the therapeutic agent is an siRNA, miRNA, shRNA, or an antisense molecule, which inhibits a targeted nucleic acid including those encoding proteins that are involved in aggravation of the pathological processes.

In one embodiment, the nucleic acid comprises a promoter/regulatory sequence such that the nucleic acid is capable of directing expression of the nucleic acid. Thus, the invention encompasses expression vectors and methods for the introduction of exogenous nucleic acid into cells with concomitant expression of the exogenous nucleic acid in the cells.

In one embodiment, siRNA is used to decrease the level of a targeted protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. Optimizing siRNAs involves consideration of overall G/C content, C/T content at the termini, Tm and the nucleotide content of the 3′ overhang.

In one aspect, the invention includes a vector comprising an siRNA or an antisense polynucleotide. In one embodiment, the siRNA or antisense polynucleotide is capable of inhibiting the expression of a target polypeptide. The incorporation of a desired polynucleotide into a vector and the choice of vectors are well-known in the art.

In certain embodiments, the expression vectors described herein encode a short hairpin RNA (shRNA) therapeutic agent. shRNA molecules are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleave the shRNA to form siRNA.

In order to assess the expression of the siRNA, shRNA, or antisense polynucleotide, the expression vector to be introduced into a cell can also contain either a selectable marker gene or a reporter gene or both to facilitate identification of expressing cells from the population of cells sought to be transfected or infected using the delivery vehicle of the invention. In other embodiments, the selectable marker may be carried on a separate piece of DNA and also be contained within the delivery vehicle. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in one aspect, the delivery vehicle may contain a vector, comprising the nucleotide sequence or the construct to be delivered. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the invention or the gene construct of the invention can be inserted include a Tet-on inducible vector for expression in eukaryote cells.

The vector may be obtained by conventional methods known by persons skilled in the art. In a particular embodiment, the vector is a vector useful for transforming animal cells.

In one embodiment, the recombinant expression vectors may also contain nucleic acid molecules, which encode a peptide or peptidomimetic.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression. The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of host cells. Suitable selectable marker genes are genes encoding proteins such as G418 and hygromycin, which confer resistance to certain drugs, β-galactosidase, chloramphenicol acetyltransferase, firefly luciferase, or an immunoglobulin or portion thereof such as the Fc portion of an immunoglobulin, such as IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like.

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queuosine, and wybutosine and the like, as well as acetyl- methyl-, thio- and other modified forms of adenine, cytidine, guanine, thymine, and uridine.

In one embodiment of the invention, an antisense nucleic acid sequence, which is expressed by a plasmid vector is used as a therapeutic agent to inhibit the expression of a target protein. The antisense expressing vector is used to transfect a mammalian cell or the mammal itself, thereby causing reduced endogenous expression of the target protein.

Antisense molecules and their use for inhibiting gene expression are well known in the art. Antisense nucleic acids are DNA or RNA molecules that are complementary, as that term is defined elsewhere herein, to at least a portion of a specific mRNA molecule. In the cell, antisense nucleic acids hybridize to the corresponding mRNA, forming a double-stranded molecule thereby inhibiting the translation of genes.

The use of antisense methods to inhibit the translation of genes is known in the art. Such antisense molecules may be provided to the cell via genetic expression using DNA encoding the antisense molecule.

Alternatively, antisense molecules of the invention may be made synthetically and then provided to the cell. In some embodiments, antisense oligomers are between about 10 to about 30, or about 15 nucleotides, since they are easily synthesized and introduced into a target cell. Synthetic antisense molecules contemplated by the invention include oligonucleotide derivatives known in the art which have improved biological activity compared to unmodified oligonucleotides.

In one embodiment of the invention, a ribozyme is used as a therapeutic agent to inhibit expression of a target protein. Ribozymes useful for inhibiting the expression of a target molecule may be designed by incorporating target sequences into the basic ribozyme structure, which are complementary, for example, to the mRNA sequence encoding the target molecule. Ribozymes targeting the target molecule, may be synthesized using commercially available reagents (Applied Biosystems, Inc., Foster City, CA) or they may be genetically expressed from DNA encoding them.

In one embodiment, the therapeutic agent may comprise one or more components of a CRISPR-Cas system, where a guide RNA (gRNA) targeted to a gene encoding a target molecule, and a CRISPR-associated (Cas) peptide form a complex to induce mutations within the targeted gene. In one embodiment, the therapeutic agent comprises a gRNA or a nucleic acid molecule encoding a gRNA. In one embodiment, the therapeutic agent comprises a Cas peptide or a nucleic acid molecule encoding a Cas peptide.

In one embodiment, the agent comprises a miRNA or a mimic of a miRNA. In one embodiment, the agent comprises a nucleic acid molecule that encodes a miRNA or mimic of a miRNA.

miRNAs are small non-coding RNA molecules that are capable of causing post-transcriptional silencing of specific genes in cells by the inhibition of translation or through degradation of the targeted mRNA. A miRNA can be completely complementary or can have a region of non-complementarity with a target nucleic acid, consequently resulting in a “bulge” at the region of non-complementarity. A miRNA can inhibit gene expression by repressing translation, such as when the miRNA is not completely complementary to the target nucleic acid, or by causing target RNA degradation, which is believed to occur only when the miRNA binds its target with perfect complementarity. The disclosure also can include double-stranded precursors of miRNA. A miRNA or pri-miRNA can be 18-100 nucleotides in length, or from 18-80 nucleotides in length. Mature miRNAs can have a length of 19-30 nucleotides, or 21-25 nucleotides, particularly 21, 22, 23, 24, or 25 nucleotides. MiRNA precursors typically have a length of about 70-100 nucleotides and have a hairpin conformation. miRNAs are generated in vivo from pre-miRNAs by the enzymes Dicer and Drosha, which specifically process long pre-miRNA into functional miRNA. The hairpin or mature microRNAs, or pri-microRNA agents featured in the disclosure can be synthesized in vivo by a cell-based system or in vitro by chemical synthesis.

In various embodiments, the agent comprises an oligonucleotide that comprises the nucleotide sequence of a disease-associated miRNA. In certain embodiments, the oligonucleotide comprises the nucleotide sequence of a disease-associated miRNA in a pre-microRNA, mature or hairpin form. In other embodiments, a combination of oligonucleotides comprising a sequence of one or more disease-associated miRNAs, any pre-miRNA, any fragment, or any combination thereof is envisioned.

miRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism.

Modifications can also increase sequence specificity, and consequently decrease off-site targeting. Methods of synthesis and chemical modifications are described in greater detail below. If desired, miRNA molecules may be modified to stabilize the miRNAs against degradation, to enhance half-life, or to otherwise improve efficacy. For increased nuclease resistance and/or binding affinity to the target, the single-stranded oligonucleotide agents featured in the disclosure can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleotide modifications can also increase binding affinity to the target. The inclusion of pyranose sugars in the oligonucleotide backbone can also decrease endonucleolytic cleavage. An oligonucleotide can be further modified by including a 3′ cationic group, or by inverting the nucleoside at the 3′-terminus with a 3-3′ linkage. In another alternative, the 3′-terminus can be blocked with an aminoalkyl group. Other 3′ conjugates can inhibit 3′-5′ exonucleolytic cleavage. While not being bound by theory, a 3′ may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

In one embodiment, the miRNA includes a 2′-modified oligonucleotide containing oligodeoxynucleotide gaps with some or all internucleotide linkages modified to phosphorothioates for nuclease resistance. The presence of methylphosphonate modifications increases the affinity of the oligonucleotide for its target RNA and thus reduces the ICSQ. This modification also increases the nuclease resistance of the modified oligonucleotide. It is understood that the methods and reagents of the present disclosure may be used in conjunction with any technologies that may be developed to enhance the stability or efficacy of an inhibitory nucleic acid molecule.

miRNA molecules include nucleotide oligomers containing modified backbones or non-natural internucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included.

A miRNA described herein, which may be in the mature or hairpin form, may be provided as a naked oligonucleotide. In some cases, it may be desirable to utilize a formulation that aids in the delivery of a miRNA or other nucleotide oligomer to cells.

In some examples, the miRNA composition is at least partially crystalline, uniformly crystalline, and/or anhydrous (e.g., less than 80, 50, 30, 20, or 10% water). In another example, the miRNA composition is in an aqueous phase, e.g., in a solution that includes water. The aqueous phase or the crystalline compositions can be incorporated into a delivery vehicle, e.g., a liposome (particularly for the aqueous phase), or a particle (e.g., a microparticle as can be appropriate for a crystalline composition). Generally, the miRNA composition is formulated in a manner that is compatible with the intended method of administration. A miRNA composition can be formulated in combination with another agent, e.g., another therapeutic agent or an agent that stabilizes an oligonucleotide agent, e.g., a protein that complexes with the oligonucleotide agent. Still other agents include chelators, e.g., EDTA (e.g., to remove divalent cations such as Mg), salts, and RNAse inhibitors (e.g., a broad specificity RNAse inhibitor). In one embodiment, the miRNA composition includes another miRNA, e.g., a second miRNA composition (e.g., a microRNA that is distinct from the first). Still other preparations can include at least three, five, ten, twenty, fifty, or a hundred or more different oligonucleotide species.

In certain embodiments, the composition comprises an oligonucleotide composition that mimics the activity of a miRNA. In certain embodiments, the composition comprises oligonucleotides having nucleobase identity to the nucleobase sequence of an miRNA, and are thus designed to mimic the activity of the miRNA. In certain embodiments, the oligonucleotide composition that mimics miRNA activity comprises a double-stranded RNA molecule which mimics the mature miRNA hairpins or processed miRNA duplexes.

In one embodiment, the oligonucleotide shares identity with endogenous miRNA or miRNA precursor nucleobase sequences. An oligonucleotide selected for inclusion in a composition of the present invention may be one of a number of lengths. Such an oligonucleotide can be from 7 to 100 linked nucleosides in length. For example, an oligonucleotide sharing nucleobase identity with a miRNA may be from 7 to 30 linked nucleosides in length. An oligonucleotide sharing identity with a miRNA precursor may be up to 100 linked nucleosides in length. In certain embodiments, an oligonucleotide comprises 7 to 30 linked nucleosides. In certain embodiments, an oligonucleotide comprises 7, 8, 9, 10, 11, 12, 13, 14, 15, 16, 17, 18, 19, 20, 21, 22, 23, 24, 25, 26, 28, 29, or 30 linked nucleotides. In certain embodiments, an oligonucleotide comprises 19 to 23 linked nucleosides. In certain embodiments, an oligonucleotide is from 40 up to 50, 60, 70, 80, 90, or 100 linked nucleosides in length.

In certain embodiments, an oligonucleotide has a sequence that has a certain identity to a miRNA or a precursor thereof. Nucleobase sequences of mature miRNAs and their corresponding stem-loop sequences described herein are the sequences found in miRBase, an online searchable database of miRNA sequences and annotation. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence. The compositions of the present invention encompass oligomeric compound comprising oligonucleotides having a certain identity to any nucleobase sequence version of a miRNAs described herein.

In certain embodiments, an oligonucleotide has a nucleobase sequence at least 60%, 65%, 70%, 75%, 80%, 85%, 90%, 95%, 97%, 98% or 99% identical to the miRNA over a region of 7, 8, 9, 10, 11, 12, 13, 14, 15, 16, 17, 18, 19, 20, 21, 22, 23, 24, 25, 26, 27, 28, 29, or 30 nucleobases. Accordingly, in certain embodiments the nucleobase sequence of an oligonucleotide may have one or more non-identical nucleobases with respect to the miRNA.

In the sense used in this description, a nucleotide sequence is “substantially homologous” to any of the nucleotide sequences describe herein when its nucleotide sequence has a degree of identity with respect to the nucleotide sequence of at least 60%, at least 70%, at least 85%, or at least 95%. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art. In certain aspects, the identity between two amino acid sequences is determined by using the BLASTN algorithm.

In one embodiment, the nucleic acid molecule is a mRNA molecule. In some embodiments, the mRNA molecule comprises a nucleotide sequence that can alternatively comprise sequence variations with respect to the original nucleotide sequences, for example, substitutions, insertions and/or deletions of one or more nucleotides, with the condition that the resulting polynucleotide encodes a polypeptide according to the invention.

In certain embodiments, the composition comprises a nucleic acid molecule encoding a miRNA, precursor, mimic, or fragment thereof. For example, the composition may comprise a viral vector, plasmid, cosmid, or other expression vector suitable for expressing the miRNA, precursor, mimic, or fragment thereof in a desired mammalian cell or tissue.

### Polypeptide

In other related aspects, the agent is a polypeptide. In various embodiments, the agent is an isolated polypeptide. In other related aspects, the agent includes an isolated polypeptide. For example, in one embodiment, the polypeptide of the invention inhibits or activates a target directly by binding to the target thereby modulating the normal functional activity of the target. In one embodiment, the polypeptide of the invention modulates the target by competing with endogenous proteins. In one embodiment, the polypeptide of the invention modulates the activity of the target by acting as a transdominant negative mutant.

The variants of the polypeptide therapeutic agents may be (i) one in which one or more of the amino acid residues are substituted with a conserved or non-conserved amino acid residue and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

As used herein, an amino acid sequence is “substantially homologous” to any of the amino acid sequences described herein when its amino acid sequence has a degree of identity with respect to the amino acid sequence of at least 60%, at least 70%, at least 85%, or at least 95%. In one aspect, the identity between two amino acid sequences is determined by using the BLASTN algorithm.

In one embodiment, the agent is a peptide. Thus, in one aspect, a peptide can be incorporated into the nanoparticle and/or the conjugate of the invention. Thus, in one embodiment, the agent is a peptide. The peptide of the present invention may be made using chemical methods. For example, peptides can be synthesized by solid phase techniques, cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

As known in the art the “similarity” between two peptides is determined by comparing the amino acid sequence and its conserved amino acid substitutes of one peptide to a sequence of a second peptide. Variants are defined to include peptide sequences different from the original sequence, for example different from the original sequence in less than 40% of residues per segment of interest, different from the original sequence in less than 25% of residues per segment of interest, different by less than 10% of residues per segment of interest, or different from the original protein sequence in just a few residues per segment of interest and at the same time sufficiently homologous to the original sequence to preserve the functionality of the original sequence. The present invention includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. In one aspect, the identity between two amino acid sequences is determined by using the BLASTP algorithm.

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts to a standard translation reaction.

The peptides of the invention may include unnatural amino acids formed by post-translational modification or by introducing unnatural amino acids during translation.

### Antibodies

In one embodiment, the agent is an antibody. Thus, in various embodiments, the composition of the invention comprises an antibody, or antibody fragment. In certain embodiments, the antibody targeting domain specifically binds to a target of interest. In some embodiments, the target of interest is a target at or near the BBB. In some embodiments, the target of interest is a target at or near the BRB. Such antibodies include polyclonal antibodies, monoclonal antibodies, Fab and single chain Fv (scFv) fragments thereof, bispecific antibodies, heteroconjugates, human and humanized antibodies.

The antibodies may be intact monoclonal or polyclonal antibodies, and immunologically active fragments (e.g., a Fab or (Fab)2 fragment), an antibody heavy chain, an antibody light chain, humanized antibodies, a genetically engineered single chain Fv molecule, or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmacokinetic activity may be generated in a bacterial expression system. Single chain Fv fragments show low immunogenicity.

The skilled artisan would appreciate, based upon the disclosure provided herein, that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods.

Monoclonal antibodies directed against full length or peptide fragments of a protein or peptide may be prepared using any well-known monoclonal antibody preparation procedures. Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology. Further, the antibody of the invention may be “humanized” using the technology described in, and other methods of humanizing antibodies well-known in the art or to be developed.

In some embodiments, a non-human antibody is humanized, where specific sequences or regions of the antibody are modified to increase similarity to an antibody naturally produced in a human or fragment thereof. A humanized antibody can be produced using a variety of techniques known in the art, veneering or resurfacing, chain shuffling. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions.

In one embodiment, the antibody fragment provided herein is a single chain variable fragment (scFv). In various embodiments, the antibodies of the invention may exist in a variety of other forms including, for example, Fv, Fab, and (Fab′) 2, as well as bi-functional (i.e. bi-specific) hybrid antibodies. In some embodiments, the antibodies and fragments thereof of the invention bind a cell bearing antigen, TCR, and/or BCR with wild-type or enhanced affinity. In some embodiments, the antibodies and fragments thereof of the invention bind a T cell bearing TCR with wild-type or enhanced affinity. In some embodiments, the antibodies and fragments thereof of the invention bind a B cell bearing BCR with wild-type or enhanced affinity. In various embodiments, a human scFv may also be derived from a yeast display library.

ScFvs can be prepared according to method known in the art. ScFv molecules can be produced by linking VH and VL regions together using flexible polypeptide linkers. The scFv molecules comprise flexible polypeptide linker (e.g., a Ser-Gly linker) with an optimized length and/or amino acid composition. The flexible polypeptide linker length can greatly affect how the variable regions of an scFv fold and interact. In fact, if a short polypeptide linker is employed (e.g., between 5-10 amino acids, intrachain folding is prevented. Interchain folding is also required to bring the two variable regions together to form a functional epitope binding site.

The scFv can comprise a polypeptide linker sequence of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The flexible polypeptide linker sequence may comprise any naturally occurring amino acid. In some embodiments, the flexible polypeptide linker sequence comprises amino acids glycine and serine. In another embodiment, the flexible polypeptide linker sequence comprises sets of glycine and serine repeats such as (Gly4Ser)n, where n is a positive integer equal to or greater than 1. In one embodiment, the flexible polypeptide linkers include, but are not limited to, (Gly4Ser)4 or (Gly4Ser)3. Variation in the flexible polypeptide linker length may retain or enhance activity, giving rise to superior efficacy in activity studies.

The invention encompasses monoclonal, synthetic antibodies, and the like. One skilled in the art would understand, based upon the disclosure provided herein, that the crucial feature of the antibody of the invention is that the antibody binds specifically with an antigen of interest. That is, the antibody of the invention recognizes an antigen of interest or a fragment thereof (e.g., an immunogenic portion or antigenic determinant thereof).

### Adjuvant

In one embodiment, the agent is an adjuvant. Thus, in various embodiments, the composition comprises an adjuvant. In one embodiment, the composition comprises a nucleic acid molecule encoding an adjuvant.

Exemplary adjuvants include, but is not limited to, alpha-interferon, gamma-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHIC, CD80, CD86 including IL-15 having the signal sequence deleted and optionally including the signal peptide from IgE. Other genes which may be useful adjuvants include those encoding: MCP-I, MIP-Ia, MIP-Ip, IL-8, RANTES, L-selectin, P-selectin, E-selectin, CD34, GlyCAM-1, MadCAM-1, LFA-I, VLA-I, Mac-1, p150.95, PECAM, ICAM-I, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-I, DR3, TRAMP, Apo-3, AIR, LARD, NGRF, DR4, DR5, KILLER, TRAIL-R2, TRICK2, DR6, Caspase ICE, Fos, c-jun, Sp-I, Ap-I, Ap-2, p38, p65Rel, MyD88, IRAK, TRAF6, IkB, Inactive NIK, SAP K, SAP-I, INK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP 1, TAP2, anti-CTLA4-sc, anti-LAG3-Ig, anti-TIM3-Ig and functional fragments thereof.

### Nucleoside-Modified RNA

In one embodiment, the agent is a nucleoside-modified RNA. Thus, in one aspect, the composition comprises a nucleoside-modified RNA. Thus, in one embodiment, the agent is a nucleoside-modified RNA In one embodiment, the composition comprises a nucleoside-modified mRNA. Nucleoside-modified mRNA have particular advantages over non-modified mRNA, including for example, increased stability, low or absent innate immunogenicity, and enhanced translation.

In certain embodiments, nucleoside-modified mRNA does not activate any pathophysiologic pathways, translates very efficiently and almost immediately following delivery, and serve as templates for continuous protein production in vivo lasting for several days. The amount of mRNA required to exert a physiological effect is small and that makes it applicable for human therapy.

In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects.

In certain embodiments, the nucleoside-modified RNA comprises the naturally occurring modified-nucleoside pseudouridine. In certain embodiments, inclusion of pseudouridine makes the mRNA more stable, non-immunogenic, and highly translatable.

It has been demonstrated that the presence of modified nucleosides, including pseudouridines in RNA suppress their innate immunogenicity. Further, protein-encoding, in vitro-transcribed RNA containing pseudouridine can be translated more efficiently than RNA containing no or other modified nucleosides. Subsequently, it is shown that the presence of pseudouridine improves the stability of RNA and abates both activation of PKR and inhibition of translation. A preparative HPLC purification procedure has been established that was critical to obtain pseudouridine-containing RNA that has superior translational potential and no innate immunogenicity. Administering HPLC-purified, pseudourine-containing RNA coding for erythropoietin into mice and macaques resulted in a significant increase of serum EPO levels, thus confirming that pseudouridine-containing mRNA is suitable for in vivo protein therapy.

The present invention encompasses RNA, oligoribonucleotide, and polyribonucleotide molecules comprising pseudouridine or a modified nucleoside. In certain embodiments, the composition comprises an isolated nucleic acid encoding an antigen or antigen binding molecule, wherein the nucleic acid comprises a pseudouridine or a modified nucleoside. In certain embodiments, the composition comprises a vector, comprising an isolated nucleic acid encoding an antigen, an antigen binding molecule, an adjuvant, or combination thereof, wherein the nucleic acid comprises a pseudouridine or a modified nucleoside.

In one embodiment, the nucleoside-modified RNA of the invention is IVT RNA. For example, in certain embodiments, the nucleoside-modified RNA is synthesized by T7 phage RNA polymerase. In another embodiment, the nucleoside-modified mRNA is synthesized by SP6 phage RNA polymerase. In another embodiment, the nucleoside-modified RNA is synthesized by T3 phage RNA polymerase.

In one embodiment, the modified nucleoside is m¹acp³Ψ (1-methyl-3-(3-amino-3-carboxypropyl) pseudouridine. In another embodiment, the modified nucleoside is m¹Ψ (1-methylpseudouridine). In another embodiment, the modified nucleoside is Ψm (2′-O-methylpseudouridine. In another embodiment, the modified nucleoside is m⁵D (5-methyldihydrouridine). In another embodiment, the modified nucleoside is m³Ψ (3-methylpseudouridine). In another embodiment, the modified nucleoside is a pseudouridine moiety that is not further modified. In another embodiment, the modified nucleoside is a monophosphate, diphosphate, or triphosphate of any of the above pseudouridines. In another embodiment, the modified nucleoside is any other pseudouridine-like nucleoside known in the art.

In another embodiment, the modified nucleoside of the present invention is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is Ψ (pseudouridine). In another embodiment, the modified nucleoside is Um (2′-O-methyluridine).

In other embodiments, the modified nucleoside is m¹A (1-methyladenosine); m²A (2-methyladenosine); Am (2′-O-methyladenosine); ms²m6A (2-methylthio-N⁶-methyladenosine); i⁶A (N⁶-isopentenyladenosine); ms²i6A (2-methylthio-N⁶isopentenyladenosine); io⁶A (N⁶-(cis-hydroxyisopentenyl)adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); fVC (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²2G (N²,N²-dimethylguanosine); m²Gm (N²,2′-O-dimethylguanosine); m²2Gm (N²,N²,2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵s²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); nCm⁵U (5-carbamoylmethyluridine); nCm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶₂A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶₂Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine).

In another embodiment, a nucleoside-modified RNA of the present invention comprises a combination of 2 or more of the above modifications. In another embodiment, the nucleoside-modified RNA comprises a combination of 3 or more of the above modifications. In another embodiment, the nucleoside-modified RNA comprises a combination of more than 3 of the above modifications.

In another embodiment, between 0.1% and 100% of the residues in the nucleoside-modified of the present invention are modified (e.g. either by the presence of pseudouridine or a modified nucleoside base). In another embodiment, 0.1% of the residues are modified. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 8%. In another embodiment, the fraction is less than 10%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, 0.1% of the residues of a given nucleoside (i.e., uridine, cytidine, guanosine, or adenosine) are modified. In another embodiment, the fraction of the given nucleotide that is modified is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction of the given nucleotide that is modified is less than 8%. In another embodiment, the fraction is less than 10%. In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, a nucleoside-modified RNA of the present invention is translated in the cell more efficiently than an unmodified RNA molecule with the same sequence. In another embodiment, the nucleoside-modified RNA exhibits enhanced ability to be translated by a target cell. In another embodiment, translation is enhanced by a factor of 2-fold relative to its unmodified counterpart. In another embodiment, translation is enhanced by a three-fold factor. In another embodiment, translation is enhanced by a 5-fold factor. In another embodiment, translation is enhanced by a 7-fold factor. In another embodiment, translation is enhanced by a 10-fold factor. In another embodiment, translation is enhanced by a 15-fold factor. In another embodiment, translation is enhanced by a 20-fold factor. In another embodiment, translation is enhanced by a 50-fold factor. In another embodiment, translation is enhanced by a 100-fold factor. In another embodiment, translation is enhanced by a 200-fold factor. In another embodiment, translation is enhanced by a 500-fold factor. In another embodiment, translation is enhanced by a 1000-fold factor. In another embodiment, translation is enhanced by a 2000-fold factor. In another embodiment, the factor is 10-1000-fold. In another embodiment, the factor is 10-100-fold. In another embodiment, the factor is 10-200-fold. In another embodiment, the factor is 10-300-fold. In another embodiment, the factor is 10-500-fold. In another embodiment, the factor is 20-1000-fold. In another embodiment, the factor is 30-1000-fold. In another embodiment, the factor is 50-1000-fold. In another embodiment, the factor is 100-1000-fold. In another embodiment, the factor is 200-1000-fold. In another embodiment, translation is enhanced by any other significant amount or range of amounts.

In another embodiment, the nucleoside-modified RNA of the present invention exhibits significantly less innate immunogenicity than an unmodified in vitro-synthesized RNA molecule with the same sequence. In another embodiment, the modified RNA molecule exhibits an innate immune response that is 2-fold less than its unmodified counterpart. In another embodiment, innate immunogenicity is reduced by a three-fold factor. In another embodiment, innate immunogenicity is reduced by a 5-fold factor. In another embodiment, innate immunogenicity is reduced by a 7-fold factor. In another embodiment, innate immunogenicity is reduced by a 10-fold factor. In another embodiment, innate immunogenicity is reduced by a 15-fold factor. In another embodiment, innate immunogenicity is reduced by a 20-fold factor. In another embodiment, innate immunogenicity is reduced by a 50-fold factor. In another embodiment, innate immunogenicity is reduced by a 100-fold factor. In another embodiment, innate immunogenicity is reduced by a 200-fold factor. In another embodiment, innate immunogenicity is reduced by a 500-fold factor. In another embodiment, innate immunogenicity is reduced by a 1000-fold factor. In another embodiment, innate immunogenicity is reduced by a 2000-fold factor. In another embodiment, innate immunogenicity is reduced by another fold difference.

In another embodiment, “exhibits significantly less innate immunogenicity” refers to a detectable decrease in innate immunogenicity. In another embodiment, the term refers to a fold decrease in innate immunogenicity (e.g., 1 of the fold decreases enumerated above). In another embodiment, the term refers to a decrease such that an effective amount of the nucleoside-modified RNA can be administered without triggering a detectable innate immune response. In another embodiment, the term refers to a decrease such that the nucleoside-modified RNA can be repeatedly administered without eliciting an innate immune response sufficient to detectably reduce production of the recombinant protein. In another embodiment, the decrease is such that the nucleoside-modified RNA can be repeatedly administered without eliciting an innate immune response sufficient to eliminate detectable production of the recombinant protein.

### Detectable Agent

In some embodiments, the agent is a detectable agent. In one embodiment, the detectable agent is a dye. In some embodiments, the agent is a diagnostic agent. In one embodiment, the diagnostic agent is a dye.

In one embodiment, the nanoparticle and/or the conjugate of the invention is bound to a dye. In one embodiment, the nanoparticle and/or the conjugate of the invention encapsulates a dye. In one embodiment, the dye is a polymethine dye. In one embodiment, the polymethine dye can be a cyanine dye, hemicyanine dye, streptocyanine dye, mercocyanine dye, oxonol dye, styryl dye, diarylmethine dye, triarylmethine dye, rylenes, squaraines, and perylene bismides and aza-analogs thereof. In one embodiment, the dye is a cyanine dye. Exemplary cyanine dyes include, but are not limited to, naphthalocyanine dyes, sulfonated indocyanines, indocyanine green, Cy3, Cy3.5, Cy5.5, and Cy7. In one embodiment, the dye is a merocyanine. Exemplary merocyanine dyes include, but are not limited to, pseudoisocyanine chloride and merocyanine I. In one embodiment, the dye is a squaraine. Exemplary squaraine dyes include, but are not limited to, squarylium dye III. In one embodiment, the dye is a rylene. Exemplary rylene dyes include, but are not limited to, bismide. In one embodiment, the dye is indocyanine green (ICG). In one embodiment, the dye is Congo Red. In one embodiment, the dye is IR783. In one embodiment, the dye is Briliant Blue G. In one embodiment, the dye is rhodamine 6G.

In one embodiment, the dye has an absorbance in the near infrared (NIR) range between about 650 and 1400 nm. In one embodiment, the dye absorbs has an absorbance in the NIR range between about 680 and 1100 nm. In one embodiment, the dye absorbs has an absorbance in the NIR range between about 700 and 950 nm. In one embodiment, the dye absorbs has an absorbance in the NIR range between about 715 and 950 nm. In one embodiment, the dye absorbs has an absorbance in the NIR range between about 790 and 895 nm.

In one embodiment, the nanoparticle and/or the conjugate of the invention comprises a dye has at least one absorption peak from about 650 to 1400 nm wavelength. In one embodiment, the nanoparticle and/or the conjugate of the invention encapsulating a dye has at least one absorption peak from about 650 to 1400 nm wavelength. In some embodiments, the absorption peak is from about 680 to 1100 nm wavelength. In some embodiments, the absorption peak is from about 715 to 950 nm wavelength. In some embodiments, the absorption peak from about 790 to 895 nm wavelength.

In one embodiment, the nanoparticle and/or the conjugate of the invention comprises at least one imaging agent. Imaging agents are materials that allow the microcarrier to be visualized after exposure to a cell or tissue. Visualization includes imaging for the naked eye, as well as imaging that requires detecting with instruments or detecting information not normally visible to the eye, and includes imaging that requires detecting of photons, sound or other energy quanta. Examples include stains, vital dyes, fluorescent markers, radioactive markers, enzymes or plasmid constructs encoding markers or enzymes. Many materials and methods for imaging and targeting may be used in nanoparticles and/or the conjugate of the invention.

Visualization based on molecular imaging typically involves detecting biological processes or biological molecules at a tissue, cell, or molecular level. Molecular imaging can be used to assess specific targets for gene therapies, cell-based therapies, and to visualize pathological conditions as a diagnostic or research tool. Imaging agents that are able to be delivered intracellularly are particularly useful because such agents can be used to assess intracellular activities or conditions. Imaging agents must reach their targets to be effective; thus, in some embodiments, an efficient uptake by cells is desirable. A rapid uptake may also be desirable to avoid the RES.

Further, imaging agents should provide high signal to noise ratios so that they may be detected in small quantities, whether directly, or by effective amplification techniques that increase the signal associated with a particular target. Amplification strategies may include, for example, avidin-biotin binding systems, trapping of converted ligands, probes that change physical behavior after being bound by a target, and taking advantage of relaxation rates. Examples of imaging technologies include magnetic resonance imaging, radionuclide imaging, computed tomography, ultrasound, and optical imaging.

Nanoparticles and/or conjugates as set forth herein may be advantageously used in various imaging technologies or strategies, for example by incorporating imaging agents into the nanoparticles and/or conjugates. Many imaging techniques and strategies are known; such strategies may be adapted to use with nanoparticles and/or conjugates. Suitable imaging agents include, for example, fluorescent molecules, labeled antibodies, labeled avidin:biotin binding agents, colloidal metals (e.g., gold, silver), reporter enzymes (e.g., horseradish peroxidase), superparamagnetic transferrin, second reporter systems (e.g., tyrosinase), and paramagnetic chelates. Advantages of nanoparticles and/or conjugates less than about 100 nm in diameter include for example, the ability of the nanoparticles and/or conjugates to be readily delivered and taken up by cells.

Compared to imaging agents that are merely conjugated to a targeting molecule, nanoparticles and/or conjugates can increase signal-to-noise ratio by delivering larger imaging agent loads per uptake event resulting in higher amplification. Many imaging agents may be loaded into the nanoparticle and/or the conjugate of the invention having a targeting molecule, which passes into cell via a single uptake.

In some embodiments, the imaging agent is a magnetic resonance imaging contrast agent. Examples of magnetic resonance imaging contrast agents include, but are not limited to, 1,4,7,10-tetraazacyclododecane-N,N′,N″N′″-tetracetic acid (DOTA), diethylenetriaminepentaacetic (DTPA), 1,4,7,10-tetraazacyclododecane-N,N′,N″,N′″-tetraethylphosphorus (DOTEP), 1,4,7,10-tetraazacyclododecane-N,N′,N″-triacetic acid (DOTA) and derivatives thereof. In some embodiments, the imaging agent is an X-Ray contrast agent. X-ray contrast agents already known in the art include a number of halogenated derivatives, especially iodinated derivatives, of 5-amino-isophthalic acid.

Clinical imaging is of increasing helpfulness in clinical and research settings. Current uses include laboratory medicine, surgery, radiation therapy, nuclear medicine, and diagnostic radiology. Nanoparticles and/or conjugates may be loaded with agents that enhance these processes, for example by enhancing contrast, or delivering agents to cells that allow for visualization with such techniques.

### Combinations

In one embodiment, the composition of the present invention comprises a combination of agents described herein. In certain embodiments, a composition comprising a combination of agents described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of agents comprises individual agents in any suitable ratio. For example, in one embodiment, the composition comprises a 1:1 ratio of two individual agents. However, the combination is not limited to any particular ratio. Rather any ratio that is shown to be effective is encompassed.

In one embodiment, the nanoparticle and/or the conjugate of the invention further comprises a targeting domain. In one aspect, the nanoparticle and/or the conjugate of the invention further comprises a targeting domain attached to the surface of the nanoparticle and/or the conjugate. In some embodiments, the targeting domain is bound to an exterior surface of the nanoparticle and/or the conjugate and recognizes a particular site of interest in a subject.

In one embodiment, the targeting domain targets at least one target (e.g., a protein) at or near the blood brain barrier or blood retina barrier. In one embodiment, the targeting domain targets at least one receptor associated with the blood brain barrier or blood retina barrier. In one embodiment, the targeting domain targets at least one cell associated with the blood brain barrier or blood retina barrier. For example, in one embodiment, the nanoparticle comprises an agent and a targeting ligand targeting at least one blood brain barrier or blood retina barrier receptor. In another embodiment, the conjugate comprises an agent and a targeting ligand targeting at least one blood brain barrier or blood retina barrier receptor. In some embodiments, the targeting ligand is an antibody that recognizes a target (e.g., a protein) at or near blood brain barrier or blood retina barrier site.

In one embodiment, the targeting domain binds to at least one cell associated with a disease or a disorder. In one embodiment, the targeting domain binds to at least one cancer cell. In one embodiment, the targeting domain binds to at least one tumor cell. In one embodiment, the targeting domain binds to at least one cancer biomarker. Examples of cancer biomarkers include, but are not limited to tumor antigens, tumor-specific antigens and tumor-associated antigens, tissue differentiation antigens, mutant protein antigens, epidermal-growth factor receptor (EGFR), oncogenic viral antigens (e.g., alphafetoprotein (AFP) and carcinoembryonic antigen (CEA)), cancer-testis antigens (e.g., CTAG1B and MAGEA1), vascular or stromal specific antigens, epithelial tumor antigen (ETA), tyrosinase, melanoma-associated antigen (MAGE), abnormal products of ras, p53, MUC-1; and tumor markers, such as AFP, carcinoma antigen (CA), CA15-3, CA27-29, CA19-9, CA-125, calcitonin, calretinin, CEA, CD34, CD99MIC 2, CD117, chromogranin, chromosomers 3, 7, 17, and 9p21, cytokeratin (e.g., TPA, TPS, Cyfra21-1, etc.), desmin, epithelial membrane antigen (EMA), factor VIII, CD31, FL1, glial fibrillary acidic protein (GFAP), gross cystic disease fluid protein (GCDFP-15), human melanoma black 45 (HMB-45), human chorionic gonadotropin (hCG), immunoglobulin, inhibin, keratin, Lactate dehydrogenase (LDH), lymphocyte marker, melanoma antigen recognized by T cells 1 (MART-1), Melan-A, myoblast determination protein 1 (Myo D1), muscle-specific actin (MSA), neurofilament, neuron-specific enolase (NSE), placental alkaline phosphatase (PLAP), prostate-specific antigen (PSA), prostatic acid phosphatase (PAP), protein tyrosine phosphatase receptor type C (PTPRC or CD45), S100 protein, smooth muscle actin (SMA), synaptophysin, thymidine kinase, thyroglobulin (Tg), thyroid transcription factor-1 (TTF-1), tumor M2-PK, and vimentin. In one embodiment, the targeting domain binds to at least one epidermal-growth factor receptor (EGFR).

In various embodiments, the targeting domain is an antibody, an antibody fragment, a peptide sequence, aptamer, folate, a ligand, a gene component, or any combination thereof. Examples of targeting domains include, but are not limited to antibodies, lymphokines, cytokines, receptor proteins such as CD4 and CD8, solubilized receptor proteins such as soluble CD4, hormones, growth factors, peptidomimetics, synthetic ligands, and the like which specifically bind desired target cells, and nucleic acids which bind corresponding nucleic acids through base pair complementarity. Targeting domains of particular interest include peptidomimetics, peptides, aptamers, folates, ligands, gene components, antibodies (e.g., monoclonal antibodies, polyclonal antibodies, recombinant antibodies, human antibodies, humanized antibodies, single domain antibodies (nanobodies), etc.) and antibody fragments (e.g., the Fab′ fragment).

In certain embodiments, the targeting domain specifically binds to a tumor-associated antigen (TAA) or tumor specific antigen (TSA). Cellular targets include tissue specific cell surface molecules, for targeting to specific sites of interest (e.g., neural cells, liver cells, bone marrow cells, kidney cells, pancreatic cells, muscle cells, and the like).

Methods of making and using antibodies are well known in the art. For example, polyclonal antibodies useful in the present invention are generated by immunizing rabbits according to standard immunological techniques well-known in the art. Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, which bind to the specific antigens of interest.

One skilled in the art would appreciate, based upon the disclosure provided herein, that the antibody can specifically bind with any portion of an antigen target, which can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Antibodies can be produced by immunizing an animal such as, but not limited to, a rabbit, a mouse or a camel, with an antigenic protein of the invention, or a portion thereof, by immunizing an animal using a protein comprising at least a portion of the antigen, or a fusion protein including a tag polypeptide portion comprising, for example, a maltose binding protein tag polypeptide portion, covalently linked with a portion comprising the appropriate amino acid residues. One skilled in the art would appreciate, based upon the disclosure provided herein, that smaller fragments of these proteins can also be used to produce antibodies that specifically bind the antigen of interest.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

Further, the skilled artisan, based upon the disclosure provided herein, would appreciate that using a non-conserved immunogenic portion can produce antibodies specific for the non-conserved region thereby producing antibodies that do not cross-react with other proteins which can share one or more conserved portions. Thus, one skilled in the art would appreciate, based upon the disclosure provided herein, that the non-conserved regions of an antigen of interest can be used to produce antibodies that are specific only for that antigen and do not cross-react non-specifically with other proteins.

In one embodiment, the targeting domain is bound directly to the nanoparticle and/or the conjugate of the invention. In one embodiment, the targeting domain is bound directly to the surface of the nanoparticle and/or the conjugate.

In one embodiment, the targeting domain is bound to the nanoparticle and/or the conjugate using a linking molecule. In one embodiment, the targeting domain is bound to the surface of the nanoparticle and/or the conjugate using a linking molecule. The linking molecules useful in the compositions and methods of the present disclosure may be any molecule capable of binding to both the coating material used in the compositions and methods of the present disclosure and the targeting domains used in the compositions and methods of the present disclosure. In certain embodiments, the linking molecule may be a hydrophilic polymer. Examples of linking molecules include, but are not limited to, poly(ethylene glycol) and its derivatives, azide compounds, maleimide compounds, hydrazine compounds, dibenzo-cyclooctyne (DBCO) compounds, dithiol compounds, dithiol compounds with hydrazide and/or carboxylic functionality, or single thiols and/or amines or their derivatives.

In certain embodiments, the linking molecule and the targeting domain may be bound by one or more covalent bonds. In certain embodiments, the linking molecule, in addition to linking the targeting domain and the coating material, may impart certain benefits upon the compositions of the present disclosure, including, but not limited to, improved hydrophilicity and stability in solution, reduced immunogenic responses upon introduction of the compositions of the present disclosure into a subject, increased circulation time of the compositions of the present disclosure when introduced into the bloodstream of a subject. The choice of a linking molecule may depend upon, among other things, the targeting domain chosen and the subject into which the compositions of the present invention are to be introduced. One of ordinary skill in the art, with the benefit of this disclosure, will recognize additional suitable linking molecules. Such linking molecules are considered to be within the spirit of the present disclosure.

In certain embodiments, the targeting domain may recognize a particular ligand or receptor present in a desired cell and/or tissue type when introduced into a subject. In certain embodiments, the targeting domain may be an antibody that recognizes such a particular ligand or receptor. The use of antibody fragments may also be suitable in the compositions of the present disclosure. The choice of a targeting domain may depend upon, among other things, the cell and/or tissue type into which an at least partial increase in uptake of the compositions of the present disclosure is desired, as well as particular ligand(s) present in such cell and/or tissue types.

In certain embodiments, the targeting domain may be chosen, among other things, to at least partially increase the uptake of the compositions of the present disclosure into a desired cell and/or tissue type when introduced into a subject. In certain embodiments, the targeting domain may be a moiety that recognizes a molecule which is present in higher amounts in an abnormal form of a tissue when compared to a normal form of the same tissue (i.e., the molecule is “up-regulated” in the abnormal form of the tissue).

In some embodiments, the suitable targeting domain may be a peptide sequence, DNA fragment, aptamer, RNA, folate, polymer, etc. One of ordinary skill in the art, with the benefit of this disclosure, will recognize other targeting domains that may be useful in the compositions of the present disclosure. Such targeting domains are considered to be within the spirit of the present disclosure.

In one embodiment, the nanoparticle and/or the conjugate of the invention comprises one or more linker molecules. Examples of linker molecules include, but are not limited to, azide functionalized molecules, maleimide functionalized molecules, carboxyl functionalized molecules, amine functionalized molecules, hydrazine functionalized molecules, dibenzo-cyclooctyne functionalized molecules, or any combination thereof.

In one embodiment, the nanoparticle and/or the conjugate of the invention comprises one or more functional groups. In some embodiments, the functional group is an azide functional group, maleimide functional group, carboxyl functional group, amine functional group, hydrazine functional group, dibenzo-cyclooctyne functional group, or any combination thereof.

### Compositions

In one aspect, the present invention relates to a composition comprising at least one nanoparticle and/or at least one conjugate of the present invention. In one aspect, the present invention relates to a composition comprising at least one nanoparticle of the present invention that selectively targets MFSD2A and/or at least one conjugate of the present invention that selectively targets MFSD2A. Thus, in certain embodiments, the composition targets at least one cell comprising MFSD2A. For example, in some embodiments, the composition targets at least one endothelial cell.

In some embodiments, the composition comprises at least two different nanoparticles comprising different agents, at least two different conjugates comprising different agents, or any combination thereof. In some embodiments, the composition comprises multiple nanoparticles, multiple conjugates, or any combination thereof.

For example, in one embodiment, the composition comprises at least two different nanoparticles comprising different agents where the first nanoparticle comprises an agent that can be a MFSD2A inhibitor and the second nanoparticle comprises a different agent that can be an additional targeting ligand that targets another receptor on the BBB or BRB.

In some embodiments, each of the nanoparticles comprises both the MFSD2A inhibitor and the additional targeting ligand. In some embodiments, the MFSD2A inhibitor and other agents can be carried by the same type of nanoparticles comprising one or more target ligands.

In another embodiment, the composition comprises at least two different conjugates comprising different agents where the first conjugate comprises an agent that can be a MFSD2A inhibitor and the second conjugate comprises a different agent that can be an additional targeting ligand that targets another receptor on the BBB or BRB.

In some embodiments, each of the conjugates comprises both the MFSD2A inhibitor and the additional targeting ligand. In some embodiments, the MFSD2A inhibitor and other agents can be bound to the same type of conjugates comprising one or more target ligands.

In another embodiment, the composition comprises at least one conjugate comprising an agent and at least one nanoparticle comprising a different agent where the conjugate comprises an agent that can be a MFSD2A inhibitor and the nanoparticle comprises a different agent that can be an additional targeting ligand that targets another receptor on the BBB or BRB.

In another embodiment, the composition comprises at least one conjugate comprising an agent and at least one nanoparticle comprising a different agent where the nanoparticle comprises an agent that can be a MFSD2A inhibitor and the conjugate comprises a different agent that can be an additional targeting ligand that targets another receptor on the BBB or BRB.

In some embodiments, both the conjugate and the nanoparticle comprise both the MFSD2A inhibitor and the additional targeting ligand.

In another embodiment, the composition comprises at least two different nanoparticles where the first nanoparticle comprises an agent that can target MFSD2A to make the BBB or BRB leaky and the second nanoparticle can cross the BBB or BRB. In another embodiment, the composition comprises at least two different conjugates where the first conjugate comprises an agent that can target MFSD2A to make the BBB or BRB leaky and the second conjugate can cross the BBB or BRB.

In another embodiment, the composition comprises at least one nanoparticle and at least one conjugate where the nanoparticle comprises an agent that can target MFSD2A to make the BBB or BRB leaky and the conjugate can cross the BBB or BRB.

In another embodiment, the composition comprises at least one nanoparticle and at least one conjugate where the conjugate comprises an agent that can target MFSD2A to make the BBB or BRB leaky and the nanoparticle can cross the BBB or BRB.

In one embodiment, the composition provided herein further comprises one or more agents that are encapsulated within, adhered to a surface of, or integrated into the structure of the nanoparticles and/or the conjugates of the invention. The one or more agents are delivered to or near the BBB or BRB site.

### Methods

In one embodiment, the present invention comprises a method of delivering an agent to a subject in need thereof. In some embodiments, the method comprises administering an amount of a composition comprising at least one nanoparticle and/or at least one conjugate of the present invention, as described above. In some embodiments, the nanoparticle is an MFSD2A-targeted nanoparticle. In some embodiments, the conjugate is an MFSD2A-targeted conjugate.

In some embodiments, the agent is one or more detectable agent. In some embodiments, the agent is one or more therapeutic agent. In some embodiments, the agent is delivered specifically to the brain of the subject. In some embodiments, the agent traverses the blood brain barrier or blood retina barrier to be delivered specifically to the brain. In some embodiments, the subject in need thereof comprises a subject at risk of developing one or more brain-associated disease or disorder. In some embodiments, the subject in need thereof comprises a subject at risk of developing one or more eye-associated disease or disorder. In some embodiments, the subject in need thereof comprises a subject suspected of having one or more brain-associated disease or disorder. In some embodiments, the subject in need thereof comprises a subject suspected of having one or more eye-associated disease or disorder. In some embodiments, the subject in need thereof comprises a subject presenting symptoms of one or more brain-associated disease or disorder. In some embodiments, the subject in need thereof comprises a subject presenting symptoms of one or more eye-associated disease or disorder. In some embodiments, the subject in need thereof comprises a subject having one or more brain-associated disease or disorder. In some embodiments, the subject in need thereof comprises a subject having one or more eye-associated disease or disorder.

In some embodiments, the methods comprise preventing, diagnosing and/or treating brain and neurodegenerative diseases or disorders in a patient. The method comprises administering an effective amount of a composition to the BBB site. The composition comprises a plurality of nanoparticles comprising one or more lipids encapsulated within, adhered to a surface of, or integrated into the structure of the nanoparticles and/or a plurality of conjugates comprising one or more lipids encapsulated within, adhered to a surface of, or integrated into the structure of the conjugates. The one or more lipids target the BBB site.

In some embodiments, the methods comprise preventing, diagnosing and/or treating eye or eye-associated diseases or disorders in a patient. The method comprises administering an effective amount of a composition to the BRB site. The composition comprises a plurality of nanoparticles comprising one or more lipids encapsulated within, adhered to a surface of, or integrated into the structure of the nanoparticles and/or a plurality of conjugates comprising one or more lipids encapsulated within, adhered to a surface of, or integrated into the structure of the conjugates. The one or more lipids target the BRB site.

In one embodiment, the present invention provides a method for gene editing of a cell of interest of a subject. For example, the method can be used to provide one or more component of a gene editing system (e.g., a component of a CRISPR system) to a cell of interest of a subject. In some embodiments, the method comprises administering to the subject a composition comprising one or more nanoparticle and/or one or more conjugate molecule formulated for targeted delivery comprising one or more nucleoside-modified RNA molecule for gene editing.

In one embodiment, the method comprises administration of the composition to a subject. In certain embodiments, the method comprises administering a plurality of doses to the subject. In another embodiment, the method comprises administering a single dose of the composition, where the single dose is effective in delivery of the target therapeutic agent.

In one aspect, the composition of the present invention comprises one or more nanoparticle and/or one or more conjugate formulated for targeted delivery of an agent to a cell of interest. Examples of such agents include, but are not limited to, a therapeutic agent, diagnostic agent, imaging agent, detectable agent, small molecule, peptide, polypeptide, amino acid molecule, nucleic acid molecule, drug, pro-drug, label, or any combination thereof.

### Diagnostic Methods

The present invention provides methods of delivering an agent to a cell of interest (e.g., a cell comprising MFSD2A) of a target subject. Exemplary cells comprising MFSD2A that can be targeted using the compositions of the invention include, but are not limited to, endothelial cells of the blood brain barrier or blood retina barrier.

In some embodiments, the agent is a diagnostic agent to detect at least one marker associated with a disease or disorder (e.g., a brain disease or disorder, neurological disease or disorder, cognitive disease or disorder, etc.). In some embodiments, the agent is an imaging agent to provide imaging data of the brain to aid in diagnosis of one or more disease or disorder.

In some embodiments, the method comprises administering to a subject at least one nanoparticle and/or at least one conjugate of the present invention, wherein the nanoparticle and/or the conjugate comprises one or more detectable agent, detecting said agent, and diagnosing said subject with a disease or disorder associated with the detectable agent or using information provided by the detectable agent to diagnoses the subject with a disease or disorder. In some embodiments, the method further comprises administering to the subject one or more therapeutic to treat said disease or disorder. In some embodiments, the nanoparticle is an MFSD2A-targeted nanoparticle. In some embodiments, the conjugate is an MFSD2A-targeted conjugate. In some embodiments, the disease or disorder is associated with the brain. Brain-associated diseases or disorders include, but are not limited to, brain tumors, neurodegenerative diseases, eating disorders, food intake disorders, schizophrenia, depression, and addiction.

### Treatment Methods

In some embodiments, the method of the present invention comprises treating a disease or disorder in a subject in need thereof by administering at least one nanoparticle and/or at least one conjugate of the present invention. In some embodiments, the nanoparticle is an MFSD2A-targeted nanoparticle. In some embodiments, the conjugate is an MFSD2A-targeted conjugate. In some embodiments, the disease or disorder is associated with the brain. Brain-associated diseases or disorders include, but are not limited to, brain tumors, neurodegenerative diseases, eating disorders, food intake disorders, schizophrenia, depression, and addiction. In some embodiments, the disease or disorder is associated with the eye.

In some embodiments, the agent is a therapeutic agent for the treatment or prevention of a disease or disorder (e.g., a brain disease or disorder, neurological disease or disorder, cognitive disease or disorder, etc.). Therefore, in some embodiments, the invention provides methods for diagnosing, treating, or preventing a disease or disorder comprising administering an effective amount of the composition comprising one or more diagnostic or therapeutic agents, one or more adjuvants, or a combination thereof.

In some embodiments, the disease or disorder is a neurological disease or disorder. In some embodiments, the neurological disease or disorder is a neurodegenerative disease or disorder. Exemplary neurodegenerative diseases or disorders include, but are not limited to, cerebrovascular accidents (CVA), Alzheimer's disease (AD), vascular-related dementia, Creutzfeldt-Jakob disease (CJD), bovine spongiform encephalopathy (BSE), Parkinson's disease (PD), brain trauma, multiple sclerosis (MS), amyotrophic lateral sclerosis (ALS), Huntington's chorea; peripheral disorders with a CNS component, such as septic shock, hepatic encephalopathy, (diabetic) hypertension, diabetic microangiopathy, sleeping sickness, Whipple disease, Duchenne muscular dystrophy (DMD), aspartylglucosaminuria, cholesterol ester storage disease, Wolman disease, cystinosis, Danon disease, Fabry disease, Farber lipogranulomatosis, Farber disease, fucosidosis, galactosialidosis types I/II, Gaucher disease types I/II/III, Gaucher disease, globoid cell leukodystrophy, Krabbe disease, glycogen storage disease II, Pompe disease, GM1-gangliosidosis types 1/11/11I, GM2-gangliosidosis type I, Tay Sachs disease, GM2-gangliosidosis type II, Sandhoff disease, GM2-gangliosidosis, α-mannosidosis types 1/11, mannosidosis, metachromatic leukodystrophy, mucolipidosis type I, sialidosis types 1/11 mucolipidosis types 11/III 1-cell disease, mucolipidosis type IIIC pseudo-Hurler polydystrophy, mucopolysaccharidosis type I, mucopolysaccharidosis type II, Hunter syndrome, mucopolysaccharidosis type IIIA, Sanfilippo syndrome, mucopolysaccharidosis type IIIB, mucopolysaccharidosis type IIIC, mucopolysaccharidosis type IIID, mucopolysaccharidosis type IVA, Morquio syndrome, mucopolysaccharidosis type IVB Morquio syndrome, mucopolysaccharidosis type VI, mucopolysaccharidosis type VII, Sly syndrome, mucopolysaccharidosis type IX, multiple sulfatase deficiency, neuronal ceroid lipofuscinosis, CLN1 Batten disease, Niemann-Pick disease, Niemann-Pick disease types A/B, C1, or C2, pycnodysostosis, Schindler disease, Schindler disease types VII, and sialic acid storage disease, pre-eclampsia; neuropsychiatric disorders, such as depression, autism, anxiety attention deficit hyperactivity disorder (ADHD), neuropsychiatric systemic lupus erythematosus, bipolar disorder, schizophrenia and other psychoses; other CNS disorders, such as brain tumors, epilepsy, migraine, narcolepsy, insomnia, chronic fatigue syndrome, mountain sickness, encephalitis, meningitis, AIDS-related dementia; and angiogenesis-related disorders, such as vascular tumors, proliferative vitreoretinopathy, rheumatoid arthritis, Crohn's disease, atherosclerosis, ovarian hyperstimulation, psoriasis, endometriosis associated with neovascularization, restenosis subsequent to balloon angioplasty, scar tissue overproduction, peripheral vascular disease, hypertension, inflammatory vasculitides, Reynaud's disease, Reynaud's phenomenon, aneurysms, arterial restenosis, thrombophlebitis, lymphangitis, lymphedema, wound healing and tissue repair, ischemia reperfusion injury, angina, myocardial infarctions, chronic heart conditions, heart failure such as congestive heart failure, age-related macular degeneration, and osteoporosis.

In some embodiments, the neurological or cognitive disease or disorder is a neuropsychiatric disorder. Examples of such neuropsychiatric disorders includes, but is not limited to, anxiety disorders, psychotic disorders, mood disorders and somatoform disorders. Other exemplary neurological or cognitive diseases or disorders include, but are not limited to, post-traumatic stress disorder (PTSD), bipolar disorder, depression, Tourette's Syndrome, schizophrenia, obsessive-compulsive disorder, generalized anxiety disorder, panic disorders, phobias, personality disorders, including antisocial personality disorder, and other disorders involving troubling memories. In one embodiment, the neurological or cognitive diseases or disorders is PTSD.

In some embodiments, the cognitive disease or disorder is an addiction or an addiction related disease or disorder in a subject. In some embodiments, the addiction includes, but is not limited to, addiction to: alcohol, tobacco, opioids, sedatives, hypnotics, anxiolytics, cocaine, Cannabis, amphetamines, hallucinogens, inhalants, phencyclidine, impulse control disorders and behavioral addictions.

In some embodiments, the disease or disorder is a brain disease or disorder, such as a brain tumor or brain cancer. Examples of such brain diseases or disorders include, but are not limited to, Astrocytomas, Meningiomas, Oligodendrogliomas, Ependymomas, Mixed gliomas, Mixed glial and neuronal tumors, Primitive neuroectodermal tumors (PNET), primary tumors of the brain, Acoustic schwannoma, Gliomas, Lymphoma, Lowest grade tumors, Pilocytic astrocytoma, Subependymal giant cell astrocytoma, Protoplasmic astrocytoma, Gangliocytoma, Ganglioglioma, Xanthomatous astrocytoma (pleomorphic xanthastrocytoma), Subependymoma, Lower grade malignancies, Fibrillary (gemistocytic, protoplasmic) astrocytoma, Ependymoma, Oligodendroglioma, Mixed oligo-astrocytoma (mixed glioma), Optic nerve glioma, Higher-grade malignancies, Anaplastic astrocytoma, Anaplastic oligodendroglioma, Anaplastic mixed glioma, Highest-grade malignancies, Glioblastoma multiforme, Gliosarcoma, Gliomatosis cerebri, Butterfly glioma, Brain stem glioma, Benign Meningioma, Atypical Meningioma, Malignant Meningioma, Carcinomatous meningitis, Primitive neuroectodermal tumors (PNET), Medulloblastoma, Ependymoblastoma, Pineoblastoma, Pituitary tumors, Pituitary region tumors, Pituitary adenoma, Pituitary carcinoma, Pseudotumor cerebri (benign intracranial hypertension), Craniopharyngioma, cyst, Rathke's cleft cyst, Pineal Tumors, Pineal cyst, Pineocytoma, Pineoblastoma, Germinoma, Germ cell tumor, Mixed germ cell tumor, Pineal gliomas, Pineal teratoma, Pituitary adenomas, secretory pituitary adenoma, Prolactinoma, Cushing's disease, Acromegaly, Hyperthyroidism Thyroid-stimulating hormone and thyroid, Choroid plexus tumors, Choroid plexus papilloma, Choroid plexus carcinoma, Neurocytoma, neurinoma, neuroblastoma, neurocytoma, Dysembroplastic neuroepithelial tumor (DNT), Lipoma, Hemangioblastoma, Hemangiopericytoma, Hamartoma, Teratoma, Tumors of nerves and/or nerve sheaths, Neuroma, Schwannoma, Neurofibroma, Cysts, Colloid cyst, Arachnoid cysts, Dermoid cyst, Epidermoid, Epidermoid cyst, Chondroma, Chordoma, Sarcomas, Gliosarcoma, Chondrosarcoma, Rhabdomyosarcoma, Primary Central Nervous System Lymphoma (PCNSL), Metastatic brain tumors, Skull base tumors, carcinomatous meningitis, spinal tumor, familial syndromes, Neurofibromatosis, Neurofibromatosis type I, Neurofibromatosis type II, Lindau Syndrome, Tuberous Sclerosis, or any combination thereof.

In some embodiments, the disease or disorder is a food intake or eating disorder. In some embodiments, the food intake disorder and eating includes, but is not limited to, obesity, metabolic disorders, Anorexia nervosa, Bulimia nervosa, Binge-eating disorder, Avoidant restrictive food intake disorder, or any combination thereof.

In some embodiments, the metabolic disorder includes, but is not limited to, 17-alpha-hydroxylase deficiency 17-beta hydroxysteroid dehydrogenase 3 deficiency, 18 Hydroxylase deficiency, 2-Hydroxyglutaric aciduria, 2-methylbutyryl-CoA dehydrogenase deficiency, 3-alpha hydroxyacyl-CoA dehydrogenase deficiency, 3-Hydroxyisobutyric aciduria, 3-methylcrotonyl-CoA carboxylase deficiency, 3-methylglutaconyl-CoA hydratase deficiency (AUH defect), 5-oxoprolinase deficiency, 6-pyruvoyl-tetrahydropterin synthase deficiency, Abdominal obesity metabolic syndrome, Abetalipoproteinemia, Acatalasemia, Aceruloplasminemia, Acetyl CoA acetyltransferase 2 deficiency, Acetyl-carnitine deficiency, Acrodermatitis enteropathica, Acromegaly, Acute intermittent Porphyria, Adenine phosphoribosyltransferase deficiency, Adenosine deaminase deficiency, Adenosine monophosphate deaminase 1 deficiency, Adenylosuccinase deficiency, Adrenomyeloneuropathy, Adult polyglucosan body disease, Albinism deafness syndrome, Albinism ocular late onset sensorineural deafness, ALG1-CDG (CDG-Ik), ALG11-CDG (CDG-Ip), ALG12-CDG (CDG-Ig), ALG13-CDG, ALG2-CDG (CDG-Ii), ALG3-CDG (CDG-Id), ALG6-CDG (CDG-Ic), ALG8-CDG (CDG-Ih), ALG9-CDG (CDG-IL), Alkaptonuria, Alpers syndrome, Alpha-1 antitrypsin deficiency, Alpha-ketoglutarate dehydrogenase deficiency, Alpha-mannosidosis, Aminoacylase 1 deficiency, Anemia due to Adenosine triphosphatase deficiency, Anemia sideroblastic and spinocerebellar ataxia, Apparent mineralocorticoid excess, Arginase deficiency, Argininosuccinic aciduria, Aromatic L-amino acid decarboxylase deficiency, Arthrogryposis renal dysfunction cholestasis syndrome, Arts syndrome, Aspartylglycosaminuria, Ataxia with oculomotor apraxia type 1, Ataxia with vitamin E deficiency, Atransferrinemia, Atypical Gaucher disease due to saposin C deficiency—See Gaucher disease, Autoimmune polyglandular syndrome type 2, Autosomal dominant neuronal ceroid lipofuscinosis 4B, Autosomal dominant optic atrophy and cataract, Autosomal dominant optic atrophy plus syndrome, Autosomal erythropoietic protoporphyria, Autosomal recessive neuronal ceroid lipofuscinosis 4A—See Adult neuronal ceroid lipofuscinosis, Autosomal recessive spastic ataxia 4, Autosomal recessive spinocerebellar ataxia 9, B4GALT1-CDG (CDG-IId), Bantu siderosis, Barth syndrome, Bartter syndrome, Bartter syndrome antenatal type 1, Bartter syndrome antenatal type 2, Bartter syndrome type 3, Bartter syndrome type 4, Beta ketothiolase deficiency, Biotin-thiamine-responsive basal ganglia disease, Biotinidase deficiency, Bjornstad syndrome, Blue diaper syndrome, Carbamoyl phosphate synthetase 1 deficiency, Carnitine palmitoyl transferase 1A deficiency, Carnitine-acylcarnitine translocase deficiency, Carnosinemia, Central diabetes insipidus, Cerebral folate deficiency, Cerebrotendinous xanthomatosis, Ceroid lipofuscinosis neuronal 1, Chanarin-Dorfman syndrome, Chediak-Higashi syndrome, CHILD syndrome, Childhood hypophosphatasia, Cholesteryl ester storage disease, Chondrocalcinosis 1, Chondrocalcinosis 2, Chondrocalcinosis due to apatite crystal deposition, Chondrodysplasia punctata 1, X-linked recessive, Chronic progressive external ophthalmoplegia, Chylomicron retention disease, Citrulline transport defect, Citrullinemia type II, COG1-CDG (CDG-IIg), COG4-CDG (CDG-IIj), COG5-CDG (CDG-IIi), COG7-CDG (CDG-IIe), COG8-CDG (CDG-IIh), Combined oxidative phosphorylation deficiency 16, Congenital bile acid synthesis defect, type 1, Congenital bile acid synthesis defect, type 2, Congenital disorder of glycosylation type I/IIX, Congenital dyserythropoietic anemia type 2, Congenital erythropoietic Porphyria, Congenital lactase deficiency, Congenital muscular dystrophy-dystroglycanopathy with or without intellectual disability (type B), Copper deficiency, familial benign, CoQ-responsive OXPHOS deficiency, Crigler Najjar syndrome, type 1, Crigler-Najjar syndrome type 2, Cystinosis, Cytochrome c oxidase deficiency, D-2-hydroxyglutaric aciduria, D-bifunctional protein deficiency, D-glycericacidemia, Danon disease, DCMA syndrome, DDOST-CDG (CDG-Ir), Deafness, dystonia, and cerebral hypomyelination, Dentatorubral-pallidoluysian atrophy, Desmosterolosis, Diamond-Blackfan anemia, Dicarboxylic aminoaciduria, Dihydrolipoamide dehydrogenase deficiency, Dihydropteridine reductase deficiency, Dihydropyrimidinase deficiency, Dihydropyrimidine dehydrogenase deficiency—Not a rare disease, Dipsogenic diabetes insipidus, DOLK-CDG (CDG-Im), Dopa-responsive dystonia, Dopamine beta hydroxylase deficiency, Dowling-Degos disease, DPAGT1-CDG (CDG-Ij), DPM1-CDG (CDG-Ie), DPM2-CDG, DPM3-CDG (CDG-Io), Dubin-Johnson syndrome, Encephalopathy due to prosaposin deficiency—See Sphingolipidosis, Erythropoietic uroporphyria associated with myeloid malignancy, Ethylmalonic encephalopathy, Fabry disease, Familial chylomicronemia syndrome, Familial HDL deficiency, Familial hypocalciuric hypercalcemia type 1, Familial hypocalciuric hypercalcemia type 2, Familial hypocalciuric hypercalcemia type 3, Familial LCAT deficiency, Familial partial lipodystrophy type 2, Fanconi Bickel syndrome, Farber disease, Fatal infantile encephalomyopathy, Fatty acid hydroxylase-associated neurodegeneration, Fish-eye disease, Fructose-1,6-bisphosphatase deficiency, Fucosidosis, Fukuyama type muscular dystrophy, Fumarase deficiency, Galactokinase deficiency, Galactosialidosis, Gamma aminobutyric acid transaminase deficiency, Gamma-cystathionase deficiency, Gaucher disease, Gaucher disease—ophthalmoplegia—cardiovascular calcification—See Gaucher disease, Gaucher disease perinatal lethal, Gaucher disease type 1, Gaucher disease type 2, Gaucher disease type 3, Gestational diabetes insipidus, Gilbert syndrome—Not a rare disease, Gitelman syndrome, Glucose transporter type 1 deficiency syndrome, Glucose-galactose malabsorption, Glutamate formiminotransferase deficiency, Glutamine deficiency, congenital, Glutaric acidemia type I, Glutaric acidemia type II, Glutaric acidemia type III, Glutathione synthetase deficiency, Glutathionuria, Glycine N-methyltransferase deficiency, Glycogen storage disease 8, Glycogen storage disease type 0, liver, Glycogen storage disease type 12, Glycogen storage disease type 13, Glycogen storage disease type 1A, Glycogen storage disease type 1, Glycogen storage disease type 3, Glycogen storage disease type 5, Glycogen storage disease type 6, Glycogen storage disease type 7, Glycoproteinosis, GM1 gangliosidosis type 1, GM1 gangliosidosis type 2, GM1 gangliosidosis type 3, GM3 synthase deficiency, GRACILE syndrome, Greenberg dysplasia, GTP cyclohydrolase I deficiency, Guanidinoacetate methyltransferase deficiency, Gyrate atrophy of choroid and retina, Haim-Munk syndrome, Hartnup disease, Hawkinsinuria, Hemochromatosis type 2, Hemochromatosis type 3, Hemochromatosis type 4, Hepatic lipase deficiency, Hepatoerythropoietic Porphyria, Hereditary amyloidosis, Hereditary coproporphyria, Hereditary folate malabsorption, Hereditary fructose intolerance, Hereditary hyperekplexia, Hereditary multiple osteochondromas, Hereditary sensory and autonomic neuropathy type 1E, Hereditary sensory neuropathy type 1, Hermansky Pudlak syndrome 2, Histidinemia, HMG CoA lyase deficiency, Homocarnosinosis, Homocysteinemia, Homocystinuria due to CBS deficiency, Homocystinuria due to MTHFR deficiency, HSD10 disease, Hurler syndrome, Hurler-Scheie syndrome, Hydroxykynureninuria, Hyper-IgD syndrome, Hyperbetaalaninemia, Hypercoagulability syndrome due to glycosylphosphatidylinositol deficiency, Hyperglycerolemia, Hyperinsulinism due to glucokinase deficiency, Hyperinsulinism-hyperammonemia syndrome, Hyperlipidemia type 3, Hyperlipoproteinemia type 5, Hyperlysinemia, Hyperphenylalaninemia due to dehydratase deficiency, Hyperprolinemia, Hyperprolinemia type 2, Hypertryptophanemia, Hypolipoproteinemia, Hypophosphatasia, I cell disease, Imerslund-Grasbeck syndrome, Iminoglycinuria, Inclusion body myopathy 2, Inclusion body myopathy 3, Infantile free sialic acid storage disease—See Free sialic acid storage disease, Infantile neuroaxonal dystrophy, Infantile onset spinocerebellar ataxia, Insulin-like growth factor I deficiency, Intrinsic factor deficiency, Isobutyryl-CoA dehydrogenase deficiency, Isovaleric acidemia, Kanzaki disease, Kearns-Sayre syndrome, Krabbe disease atypical due to Saposin A deficiency, L-2-hydroxyglutaric aciduria, L-arginine:glycine amidinotransferase deficiency, Lactate dehydrogenase A deficiency, Lactate dehydrogenase deficiency, Lathosterolosis, LCHAD deficiency, Leber hereditary optic neuropathy, Leigh syndrome, French Canadian type, Lesch Nyhan syndrome, Leucine-sensitive hypoglycemia of infancy, Leukoencephalopathy-dystonia-motor neuropathy, Leukoencephalopathy with brain stem and spinal cord involvement and lactate elevation, Limb-girdle muscular dystrophy type 2I, Limb-girdle muscular dystrophy type 2K—See Limb-girdle muscular dystrophy, Limb-girdle muscular dystrophy type 2M—See Limb-girdle muscular dystrophy, Limb-girdle muscular dystrophy type 2N—See Limb-girdle muscular dystrophy, Limb-girdle muscular dystrophy type 2O—See Limb-girdle muscular dystrophy, Limb-girdle muscular dystrophy type 2T—See Limb-girdle muscular dystrophy, Limb-girdle muscular dystrophy, type 2C, Lipase deficiency combined, Lipoic acid synthetase deficiency, Lipoid proteinosis of Urbach and Wiethe, Lowe oculocerebrorenal syndrome, Lysinuric protein intolerance, Malonyl-CoA decarboxylase deficiency, MAN1B1-CDG, Mannose-binding lectin protein deficiency—Not a rare disease, Mannosidosis, beta A, lysosomal, Maternal hyperphenylalaninemia, Maternally inherited diabetes and deafness, Medium-chain acyl-coenzyme A dehydrogenase deficiency, Megaloblastic anemia due to dihydrofolate reductase deficiency, Menkes disease, Metachromatic leukodystrophy, Metachromatic leukodystrophy due to saposin B deficiency, Methionine adenosyltransferase deficiency, Methylcobalamin deficiency cbl G type, Methylmalonic acidemia with homocystinuria type cblC—See Methylmalonic acidemia with homocystinuria, Methylmalonic acidemia with homocystinuria type cblD—See Methylmalonic acidemia with homocystinuria, Methylmalonic acidemia with homocystinuria type cblF—See Methylmalonic acidemia with homocystinuria, Methylmalonic acidemia with homocystinuria type cblJ—See Methylmalonic acidemia with homocystinuria, Methylmalonic aciduria, cblA type—See Adenosylcobalamin deficiency, Methylmalonic aciduria, cblB type—See Adenosylcobalamin deficiency, Mevalonic aciduria, MGAT2-CDG (CDG-IIa), Mild phenylketonuria, Mitochondrial complex I deficiency, Mitochondrial complex II deficiency, Mitochondrial complex III deficiency, Mitochondrial DNA depletion syndrome, encephalomyopathic form with methylmalonic aciduria, Mitochondrial DNA-associated Leigh syndrome, Mitochondrial encephalomyopathy lactic acidosis and stroke-like episodes, Mitochondrial myopathy and sideroblastic anemia, Mitochondrial myopathy with diabetes, Mitochondrial myopathy with lactic acidosis, Mitochondrial neurogastrointestinal encephalopathy syndrome, Mitochondrial trifunctional protein deficiency, MOGS-CDG (CDG-IIb), Mohr-Tranebjaerg syndrome, Molybdenum cofactor deficiency, Monogenic diabetes—Not a rare disease, Morquio syndrome B, MPDU1-CDG (CDG-If), MPI-CDG (CDG-Ib), MPV17-related hepatocerebral mitochondrial DNA depletion syndrome, Mucolipidosis III alpha/beta, Mucolipidosis type 4, Mucopolysaccharidosis type II, Mucopolysaccharidosis type III, Mucopolysaccharidosis type IIIA, Mucopolysaccharidosis type IIIB, Mucopolysaccharidosis type IIIC, Mucopolysaccharidosis type HID, Mucopolysaccharidosis type IVA, Mucopolysaccharidosis type VI, Mucopolysaccharidosis type VII, Multiple congenital anomalies-hypotonia-seizures syndrome, Multiple congenital anomalies-hypotonia-seizures syndrome type 2, Multiple endocrine neoplasia type 2B, Multiple sulfatase deficiency, Multiple symmetric lipomatosis, Muscle eye brain disease, Muscular dystrophy, congenital, megaconial type, Muscular phosphorylase kinase deficiency, Musculocontractural Ehlers-Danlos syndrome, Myoclonic epilepsy with ragged red fibers, Myoglobinuria recurrent, N acetyltransferase deficiency, N-acetyl-alpha-D-galactosaminidase deficiency type III, N-acetylglutamate synthase deficiency, NBIA/DYT/PARK-PLA2G6, Neonatal adrenoleukodystrophy, Neonatal hemochromatosis, Neonatal intrahepatic cholestasis caused by citrin deficiency, Nephrogenic diabetes insipidus, Neu Laxova syndrome, Neuroferritinopathy, Neuronal ceroid lipofuscinosis 10, Neuronal ceroid lipofuscinosis 2, Neuronal ceroid lipofuscinosis 3, Neuronal ceroid lipofuscinosis 5, Neuronal ceroid lipofuscinosis 6, Neuronal ceroid lipofuscinosis 7, Neuronal ceroid lipofuscinosis 9, Neuropathy ataxia retinitis pigmentosa syndrome, Neutral lipid storage disease with myopathy, Niemann-Pick disease type A, Niemann-Pick disease type B, Niemann-Pick disease type C1, Niemann-Pick disease type C2, Northern epilepsy, Not otherwise specified 3-MGA-uria type, Occipital horn syndrome, Ocular albinism type 1, Oculocutaneous albinism type 1, Oculocutaneous albinism type 1B, Oculocutaneous albinism type 2, Oculocutaneous albinism type 3, OPA3 defect, Optic atrophy 1, Ornithine transcarbamylase deficiency, Ornithine translocase deficiency syndrome, Orotic aciduria type 1, Papillon Lefevre syndrome, Parkinson disease type 9, Paroxysmal nocturnal hemoglobinuria, Pearson syndrome, Pentosuria, Permanent neonatal diabetes mellitus, Peroxisomal biogenesis disorders, Peroxisome disorders—Not a rare disease, Perrault syndrome, Peters plus syndrome, PGM1-CDG, Phosphoglycerate kinase deficiency, Phosphoglycerate mutase deficiency, Phosphoribosylpyrophosphate synthetase superactivity, PMM2-CDG (CDG-Ia), Pontocerebellar hypoplasia type 6, Porphyria cutanea tarda, Primary carnitine deficiency, Primary hyperoxaluria type 1, Primary hyperoxaluria type 2, Primary hyperoxaluria type 3, Primary hypomagnesemia with secondary hypocalcemia, Progressive external ophthalmoplegia, autosomal recessive 1, Progressive familial intrahepatic cholestasis 1, Progressive familial intrahepatic cholestasis type 2, Progressive familial intrahepatic cholestasis type 3, Prolidase deficiency, Propionic acidemia, Pseudocholinesterase deficiency, Pseudoneonatal adrenoleukodystrophy, Purine nucleoside phosphorylase deficiency, Pycnodysostosis, Pyridoxal 5′-phosphate-dependent epilepsy, Pyridoxine-dependent epilepsy, Pyruvate carboxylase deficiency, Pyruvate dehydrogenase complex deficiency, Pyruvate dehydrogenase phosphatase deficiency, Pyruvate kinase deficiency, Refsum disease, Refsum disease with increased pipecolic acidemia, Refsum disease, infantile form, Renal glycosuria, Renal hypomagnesemia 2, Renal hypomagnesemia-6, Renal tubulopathy, diabetes mellitus, and cerebellar ataxia due to duplication of mitochondrial DNA, RFT1-CDG (CDG-In), Rhizomelic chondrodysplasia punctata type 3—See Rhizomelic chondrodysplasia punctata, Rotor syndrome, Saccharopinuria, Salla disease—See Free sialic acid storage disease, Sarcosinemia, Scheie syndrome, Schimke immunoosseous dysplasia, Schindler disease type 1, Schneckenbecken dysplasia, SCOT deficiency, Sea-Blue histiocytosis, Sengers syndrome, Sensory ataxic neuropathy, dysarthria, and ophthalmoparesis, Sepiapterin reductase deficiency, Severe combined immunodeficiency, Short-chain acyl-CoA dehydrogenase deficiency, Sialidosis type I, Sialidosis, type II, Sialuria, French type, Sitosterolemia, Sjogren-Larsson syndrome, SLC35A1-CDG (CDG-IIf), SLC35A2-CDG, SLC35C1-CDG (CDG-IIc), Smith-Lemli-Opitz syndrome, Spastic paraplegia 7, Spinocerebellar ataxia 28, Spinocerebellar ataxia autosomal recessive 3, Spondylocostal dysostosis 1—See Spondylocostal dysostosis, Spondylocostal dysostosis 2—See Spondylocostal dysostosis, Spondylocostal dysostosis 3—See Spondylocostal dysostosis, Spondylocostal dysostosis 4—See Spondylocostal dysostosis, Spondylocostal dysostosis 6—See Spondylocostal dysostosis, Spondylodysplastic Ehlers-Danlos syndrome, Spondyloepimetaphyseal dysplasia joint laxity, Spondylothoracic dysostosis, SRD5A3-CDG (CDG-Iq), SSR4-CDG, Succinic semialdehyde dehydrogenase deficiency, Tangier disease, Tay-Sachs disease, Thiamine responsive megaloblastic anemia syndrome, Thiopurine S methyltranferase deficiency, Tiglic acidemia, TMEM165-CDG (CDG-IIk), Transaldolase deficiency, Transcobalamin 1 deficiency, Transient neonatal diabetes mellitus, Trehalase deficiency, Trimethylaminuria, Triosephosphate isomerase deficiency, Tyrosine hydroxylase deficiency, Tyrosine-oxidase temporary deficiency, Tyrosinemia type 1, Tyrosinemia type 2, Tyrosinemia type 3, Urea cycle disorders, Valinemia, Variegate Porphyria, VLCAD deficiency, Walker-Warburg syndrome, Wilson disease, Wolfram syndrome, Wolman disease, Wrinkly skin syndrome, X-linked adrenoleukodystrophy, X-linked cerebral adrenoleukodystrophy, X-linked Charcot-Marie-Tooth disease type 5—See Charcot-Marie-Tooth disease, X-linked creatine deficiency, X-linked dominant chondrodysplasia punctata 2, X-linked sideroblastic anemia, Xanthinuria type 1, Xanthinuria type 2, and Zellweger syndrome.

In some embodiments, the disease or disorder is schizophrenia. In some embodiments, the disease or disorder is depression.

In some embodiments, the disease or disorder is associated with the eye. Examples of diseases and disorders associated with the eye include, but are not limited to, Retinal Detachment, Retinitis Pigmentosa, Retinoblastoma, Retinopathy of Prematurity, Refractive Errors, Age-Related Macular Degeneration, Diabetic Retinopathy, Cataract, Amblyopia, Glaucoma, Strabismus, Astigmatism, Color Blindness, Dry Eye, Floaters, Pink Eye, Retinal Detachment, Farsightedness (Hyperopia), Nearsightedness (Myopia), Rare Diseases, Retinal Detachment, Retinitis Pigmentosa, Retinoblastoma, Retinopathy of Prematurity

In various embodiments, the disease or disorder is a disease or disorder associated with the level or activity of MFSD2A.

In one embodiment, the method comprises administering a composition of the invention comprising one or more agents for treatment or prevention of a disease or disorder (e.g., a brain disease or disorder, neurological disease or disorder, cognitive disease or disorder, etc.). In one embodiment, the compositions of the invention can be administered in combination with one or more additional therapeutic agent, an adjuvant, or a combination thereof. In one embodiment, the method comprises administering a single composition comprising one or more agents for targeted administration to a cell of interest (e.g., a cell comprising MFSD2A, endothelial cell, etc.) and a nucleic acid molecule encoding one or more adjuvants. In certain embodiments, the method comprises administering to subject a plurality of nanoparticles of the invention comprising one or more agents to a cell of interest (e.g., a cell comprising a MFSD2A, endothelial cell, etc.) and/or a plurality of conjugates of the invention comprising one or more agents to a cell of interest (e.g., a cell comprising a MFSD2A, endothelial cell, etc.).

Administration of the compositions of the invention in a method of treatment can be achieved in a number of different ways, using methods known in the art. In one embodiment, the method of the invention comprises systemic administration of the subject, including for example enteral or parenteral administration. In certain embodiments, the method comprises intradermal delivery of the composition. In another embodiment, the method comprises intravenous delivery of the composition. In some embodiments, the method comprises intramuscular delivery of the composition. In one embodiment, the method comprises subcutaneous delivery of the composition. In one embodiment, the method comprises inhalation of the composition. In one embodiment, the method comprises intranasal delivery of the composition.

In some embodiments, administration comprises intravenous, intranasal, or transdermal delivery of the nanoparticles, conjugates, or compositions of the present invention.

It will be appreciated that the composition of the invention may be administered to a subject either alone, or in conjunction with another agent.

The therapeutic and prophylactic methods of the invention thus encompass the use of pharmaceutical compositions comprising at least one nanoparticle and/or at least one conjugate of the invention comprising an agent (e.g., a therapeutic agent, an mRNA molecule, etc.) described herein, to practice the methods of the invention. The pharmaceutical compositions useful for practicing the invention may be administered to deliver a dose of from ng/kg/day and 100 mg/kg/day. In one embodiment, the invention envisions administration of a dose which results in a concentration of the compound of the present invention from 10 nM and 10 μM in a mammal.

Typically, dosages which may be administered in a method of the invention to a mammal, for example a human, range in amount from 0.01 g to about 50 mg per kilogram of body weight of the mammal, while the precise dosage administered will vary depending upon any number of factors, including but not limited to, the type of mammal and type of disease state being treated, the age of the mammal and the route of administration. In one embodiment, the dosage of the compound will vary from about 0.1 g to about 10 mg per kilogram of body weight of the mammal. In one embodiment, the dosage will vary from about 1 g to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In certain embodiments, administration of a composition of the present invention may be performed by single administration or boosted by multiple administrations.

In one embodiment, the invention includes a method comprising administering a combination of compositions described herein. In certain embodiments, the combination has an additive effect, wherein the overall effect of the administering the combination is approximately equal to the sum of the effects of administering each composition. In other embodiments, the combination has a synergistic effect, wherein the overall effect of administering the combination is greater than the sum of the effects of administering each composition.

In some aspects of the invention, the method provides for delivery of compositions for gene editing or genetic manipulation to a target cell (e.g., a cell comprising a MFSD2A, endothelial cell, etc.) of a subject to treat or prevent a disease or disorder (e.g., a brain disease or disorder, neurological disease or disorder, cognitive disease or disorder, or a disease or disorder associated with the level or activity of at least one MFSD2A or a fragment thereof).

In one aspect, the therapeutic compounds or compositions of the invention may be administered prophylactically (i.e., to prevent disease or disorder, such as a brain disease or disorder, neurological disease or disorder, cognitive disease or disorder, or a disease or disorder associated with the level or activity of at least one MFSD2A or a fragment thereof) or therapeutically (i.e., to treat disease or disorder, such as a disease or disorder associated with fibrosis, a neurological disease or disorder, a skin condition, a cancer or disease or disorder associated therewith, or a disease or disorder associated with the level or activity of at least one MFSD2A or a fragment thereof) to subjects suffering from or at risk of (or susceptible to) developing the disease or disorder (e.g., a brain disease or disorder, neurological disease or disorder, cognitive disease or disorder, an eye disease or disorder, or a disease or disorder associated with the level or activity of at least one MFSD2A or a fragment thereof). Such subjects may be identified using standard clinical methods.

In the context of the present invention, prophylactic administration occurs prior to the manifestation of overt clinical symptoms of disease or disorder (e.g., a brain disease or disorder, neurological disease or disorder, cognitive disease or disorder, or a disease or disorder associated with the level or activity of at least one MFSD2A or a fragment thereof), such that the disease or disorder is prevented or alternatively delayed in its progression. In the context of the field of medicine, the term “prevent” encompasses any activity which reduces the burden of mortality or morbidity from a disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

The composition of the invention can be useful in combination with therapeutic, anti-cancer, and/or radiotherapeutic agents. Thus, the present disclosure provides a combination of the present nanoparticle and/or conjugate with therapeutic, anti-cancer, and/or radiotherapeutic agents for simultaneous, separate, or sequential administration. The composition of the invention and the other anticancer agent can act additively or synergistically.

The therapeutic agent, anti-cancer agent, and/or radiation therapy can be administered according to therapeutic protocols well known in the art. It will be apparent to those skilled in the art that the administration of the therapeutic agent, anti-cancer agent, and/or radiation therapy can be varied depending on the disease being treated and the known effects of the anti-cancer agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., anti-neoplastic agent or radiation) on the patient, and in view of the observed responses of the disease to the administered therapeutic agents, and observed adverse effects.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. In one embodiment, such powders comprise particles wherein at least 98% of the particles by weight have a diameter greater than 0.5 nanometers and at least 95% of the particles by number have a diameter less than 7 nanometers. In one embodiment, at least 95% of the particles by weight have a diameter greater than 1 nanometer and at least 90% of the particles by number have a diameter less than 6 nanometers. In one embodiment, dry powder compositions include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in some aspects, having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the certain embodiments of the present invention, and are not to be construed as limiting in any way the remainder of the disclosure.

### Example 1: Brain Targeted Nanoparticles for Delivering Agents to the Brain

Major facilitator superfamily domain-containing protein-2a (MFSD2A), a membrane transport protein expressed on the endothelial cells of the BBB, plays a crucial role in the formation and function of the BBB. It is responsible for transport lysophosphatidylcholines and docosahexaenoic acid across the BBB. The lipid transport function of MFSD2A is also essential for the uptake of polyunsaturated fatty acids into endothelial cells for BBB formation. Recent studies indicated when Mfsd2a expression is high, endothelial transcytosis of the BBB is low. When MFSD2A expression is low or knocked out, the BBB is leaky via enhanced transcytosis. The blood brain barrier (BBB) limits the delivery of various therapeutic agents and diagnostic agents to the brain, which hinder the effectiveness of imaging of the brain and treatment of neurological or cognitive diseases or disorders.

The present studies disclose the design and development of nanoparticles targeting major facilitator superfamily domain-containing protein-2a (MFSD2A), a membrane transport protein in the brain. There are no previously known nanoparticles that target MFSD2A. MFSD2A is expressed abundantly and specifically on the endothelial cells of the blood brain barrier (BBB). The present studies have utilized nanoparticles comprising ligands of MFSD2A to specifically target the nanoparticles to cross the BBB. As such, the herein-described nanoparticles can carry diagnostic molecules for imaging of the brain as well as therapeutic agents for treating brain disorders. The nanoparticles can also carry inhibitors to enhance BBB permeability.

More specifically, the herein-described nanoparticles were coated with one or more ligands of MFSD2A, such as acyl-carnitine and fatty acid-lysophosphatidylcholine (LysoPC), especially DHA-LysoPC. MFSD2A is a specific receptor on endothelial cells of the BBB regulating transcytosis and thus the herein-described nanoparticles were specifically targeted to the brain. The nanoparticles were able to cross the BBB by (a) inhibiting MFSD2A leading to restrictive transcytosis; (b) using the transport function of MFSD2A to deliver across the BBB; and (c) by prolonging the time of interaction of drugs with the BBB to increase the possibility of penetration. Moreover, the herein-described nanoparticles can deliver preventative, diagnostic, therapeutic, and bioactive agents to the brain with greater efficiency and lesser toxicity. The nanoparticles can be administered via intravenous injection, intranasal route, transdermal patches. As such, the nanoparticles can be used for various applications, such as the treatment or prevention of neurodegenerative diseases, opioids overdose, and food intake disorders as well as temperature and mood regulation.

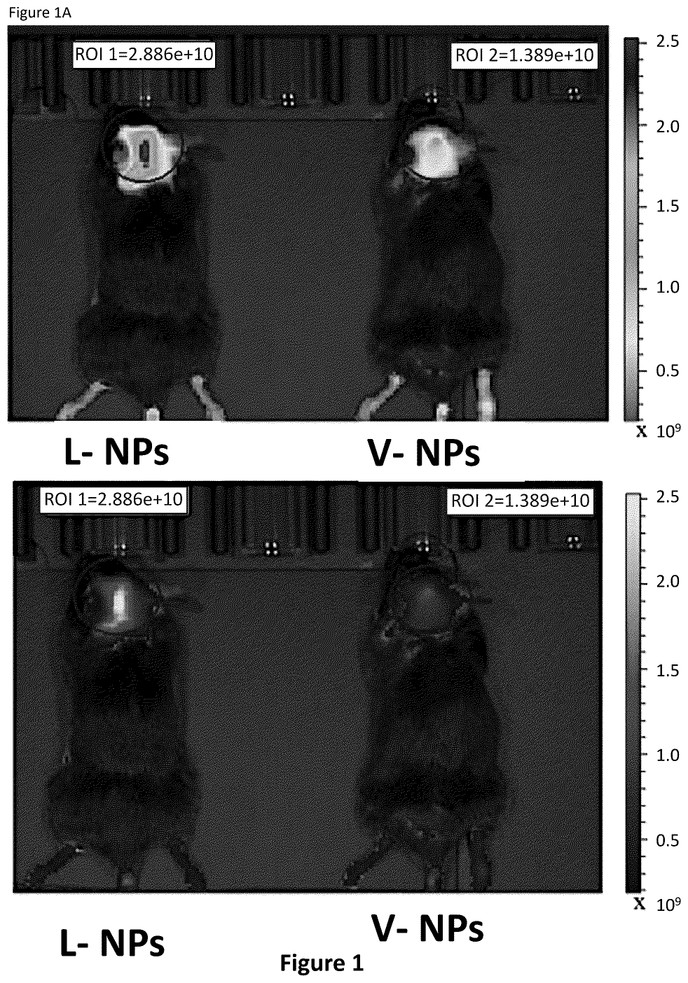

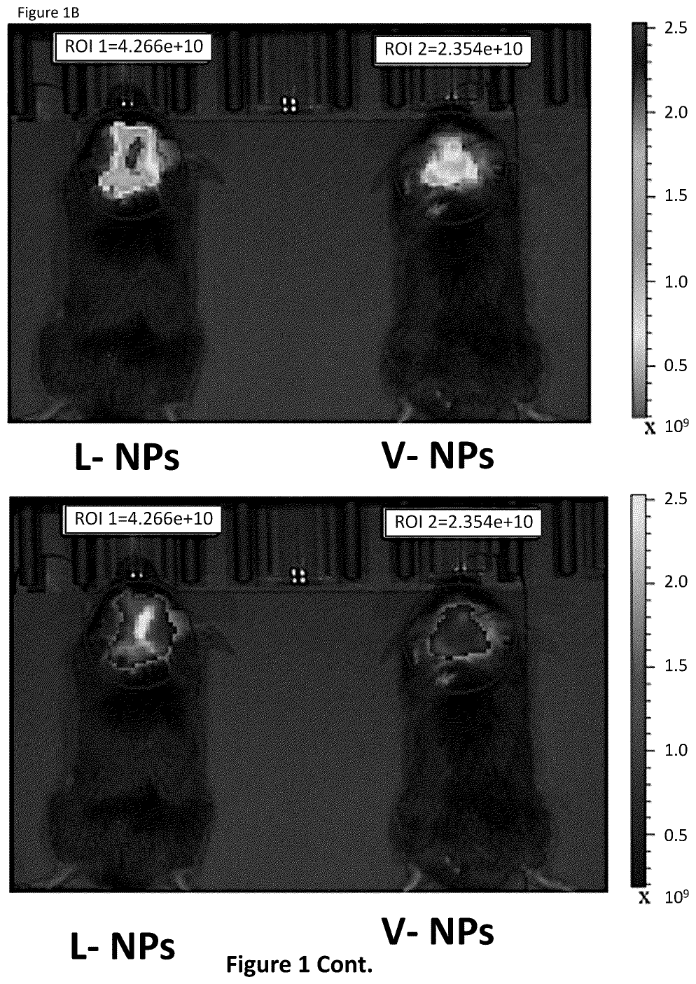

In exemplary experiments, non-targeted nanoparticles (V-NPs) comprised of soy phosphatidylcholine (PC), alpha-tocopherol acetate (αTA), Kolliphor® HS15 (macrogol (15)-hydroxystearate), and an imaging dye were generated. MFSD2A-targeted nanoparticles (L-NPs) comprised of soy phosphatidylcholine (PC), alpha-tocopherol acetate (aTA), Kolliphor® HS15, lysophosphatidylcholine-DHA (MFSD2A target ligand), and an imaging dye were also generated. The size of nanoparticles was between 30 and 100 nm, with an average diameter of around 50 nm. The non-encapsulated (free) dye was removed, and the nanoparticles were passed through a 0.22 μm filter to make them sterile. Dye intensity of non-targeted nanoparticles and MFSD2A-targeted nanoparticles was measured to ensure that the two types of particles carried the same amount of dye before treating mice with an equivalent mass of nanoparticles. Young adult C57BL/6J mice were intravenously administered non-targeted nanoparticles (V-NPs) or MFSD2A-targeted nanoparticles (L-NPs) via tail vein injection. Three hours after injection, the mice treated with MFSD2A-targeted nanoparticles as compared to non-targeted nanoparticles had almost 2-fold higher dye signal intensities in the brain (FIG. 1A). Sixteen hours after injection, a similar difference was observed in the brain, with considerably higher dye signal intensities in the L-NP treated mice (FIG. 1).

### Example 2: Targeted Delivery of Small Molecules to the Brain by Nanoparticles

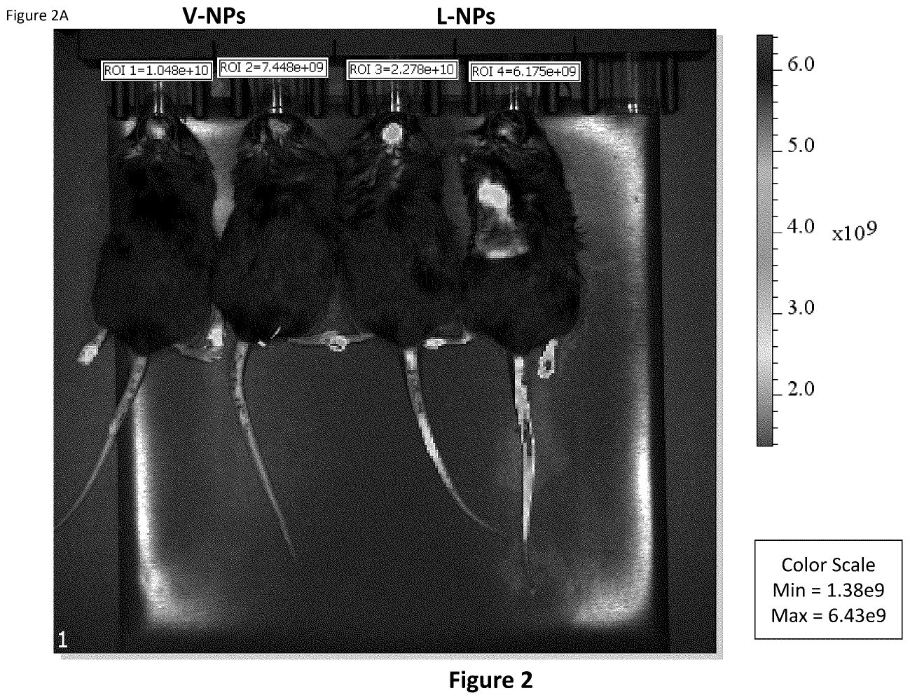

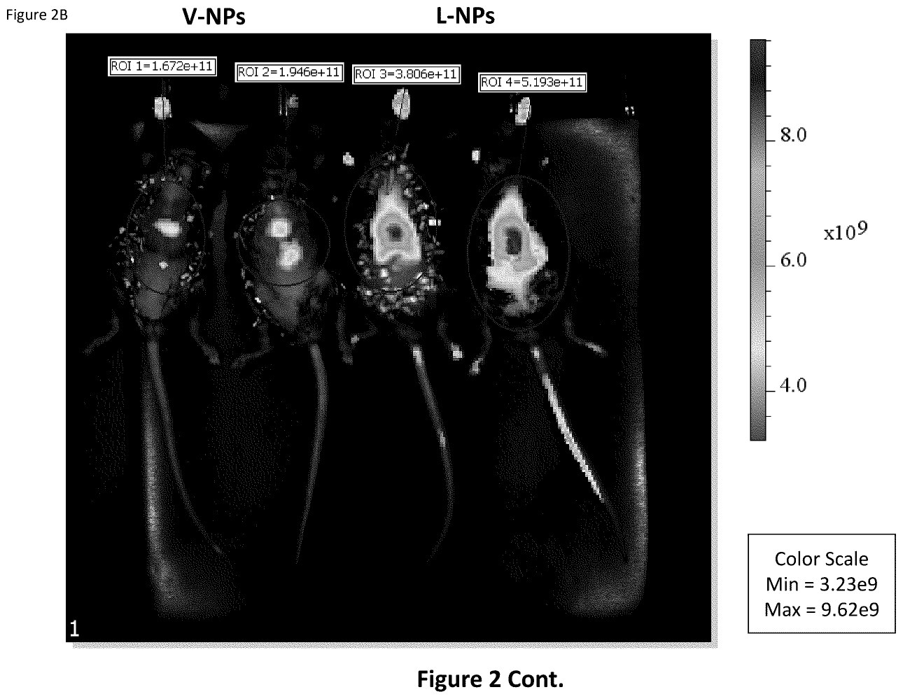

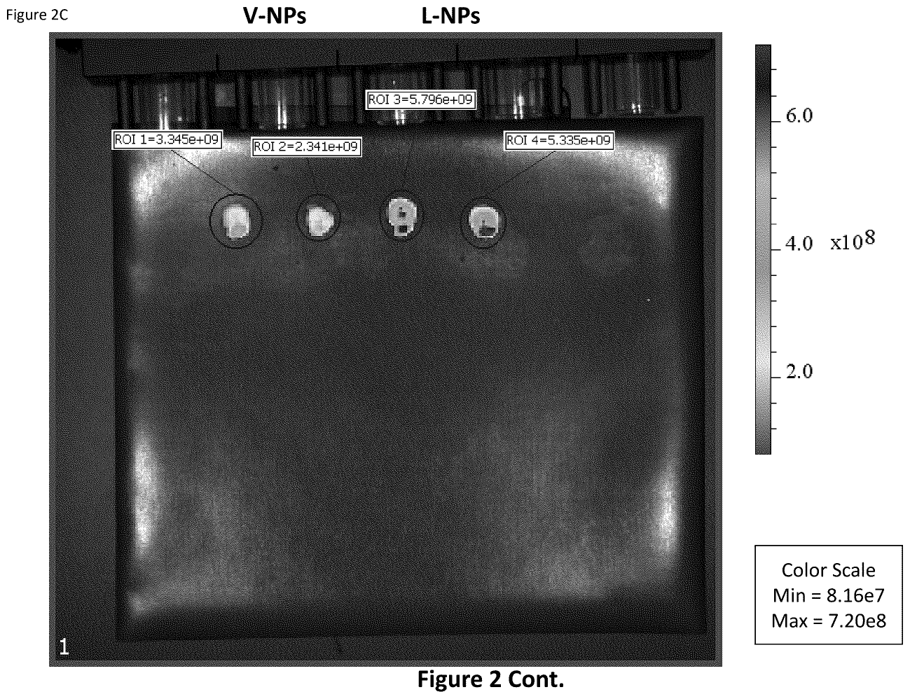

Non-targeted nanoparticles (V-NPs) were prepared as described above. MFSD2A-targeted nanoparticles were prepared in a similar manner with PC, αTC, Kolliphor® HS15, lysophosphatidylcholine-DHA, and dye as above with the inclusion of epigallocatechin gallate and trans-resveratrol. The nanoparticles were of the same size and dye content as those previously prepared and sterilized in the same manner. The encapsulation efficiency of the nanoparticles was also examined and found to be more than 90% for dye, epigallocatechin gallate and trans-resveratrol. Nanoparticles were delivered to C57BL/6J mice via tail injection. Delivery of dye via the nanoparticles was similar to that previously observed (FIGS. 2A and 2B). The isolated brains of sacrificed animals were also compared for dye delivery under an in vivo imaging system (FIG. 2C).

### Example 3: Brain Targeted Liposomes for Delivery of Agents to the Brain

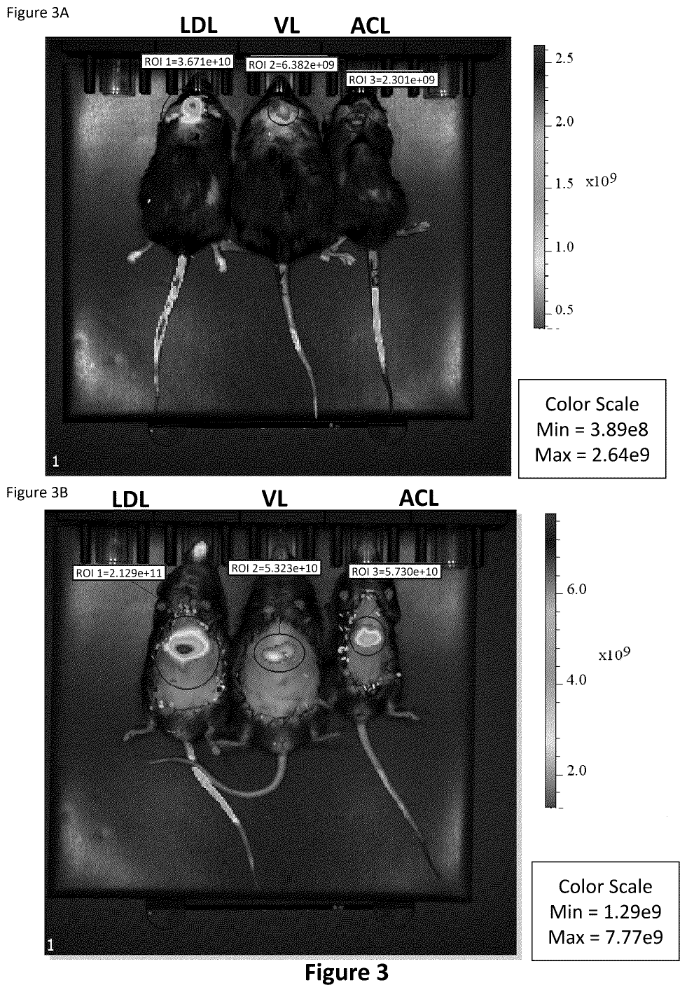

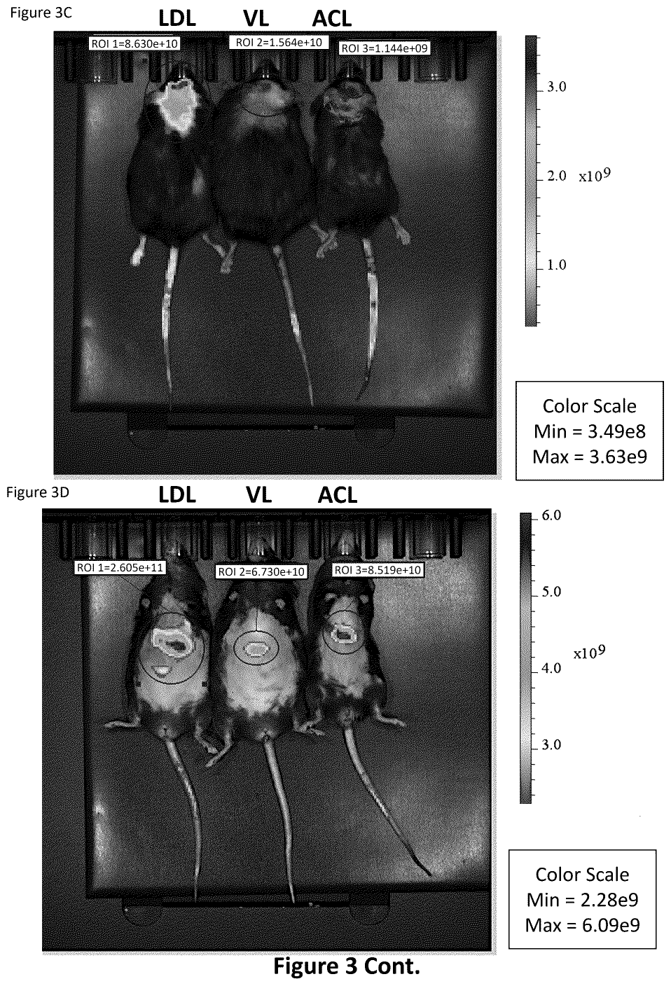

To investigate the targeting capabilities in a variety of carriers, liposomes were prepared. Non-targeted liposomes (V-Lipos) were prepared from PC, cholesterol, and dye. MFSD2A-targeted liposomes (L-Lipos) were prepared from PC, cholesterol, lysophosphatidylcholine-DHA, and dye. The liposomes produced had a diameter of about 100 nm. Dye encapsulation efficiency was more than 90%. Non-encapsulated dye was removed, and the liposomes were passed through a 0.22 μm filter to make them sterile. Liposome loading was found to be equivalent between targeted and non-targeted liposomes. Either non-targeted or MFSD2A-targeted liposomes were delivered to C57BL/6J mice via tail vein injection. One hour after injection, mice treated with Lyso-DHA-liposomes (LDLs) had approximately four-fold increased dye signal intensities in the brain relative to those given untargeted liposomes (VLs)(FIG. 3A). Abdominal dye signal intensities were approximately 2-fold higher in LDL-treated mice relative to VL-treated mice (FIG. 3B). The increase in dye intensity in LDL-treated mice was similarly increased after 18 hours with ACL-treated mice maintaining decreased delivery (FIGS. 3C and 3D).

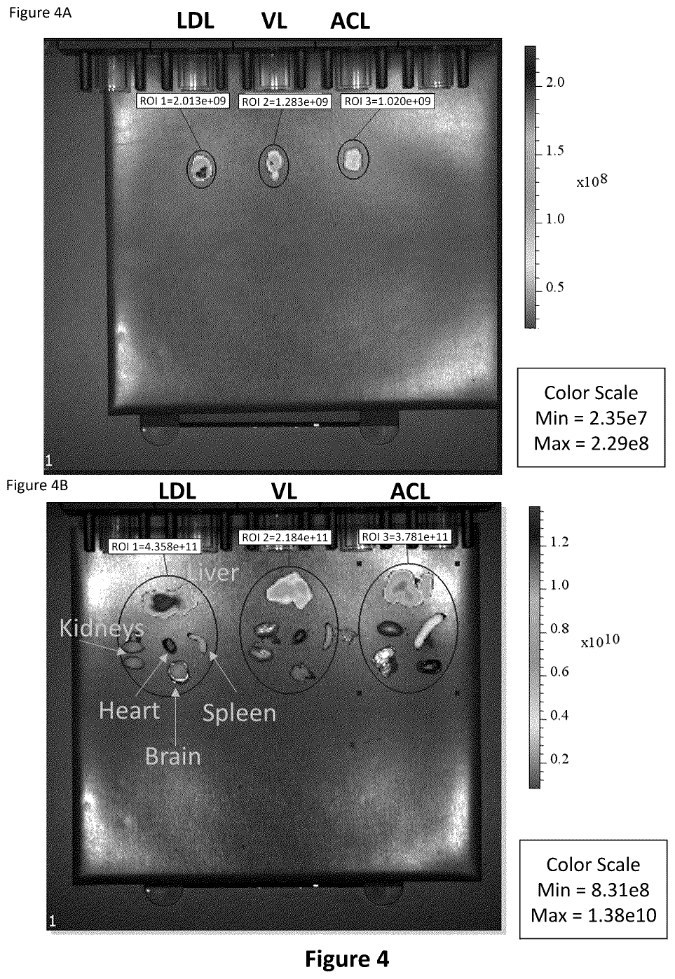

After 18 hours, the mice were sacrificed, and their organs were harvested. Isolated brains of LDL-treated mice yielded an approximately 2-fold increased dye intensity relative to both VL-treated mice (FIG. 4A). Delivery dye to liver, kidneys, and heart were all found to be increased in LDL-treated mice relative to VL-treated mice (FIG. 4B).

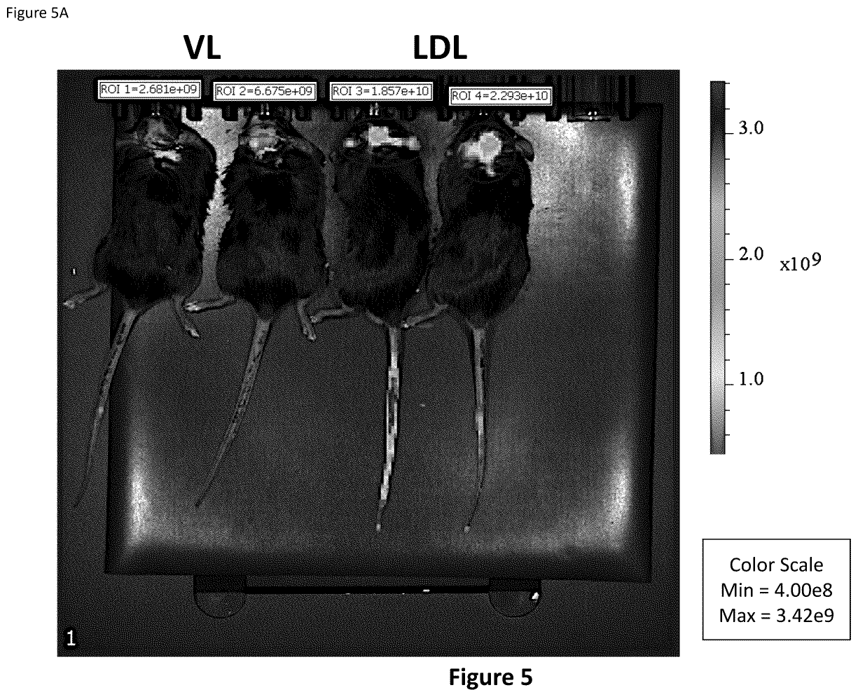

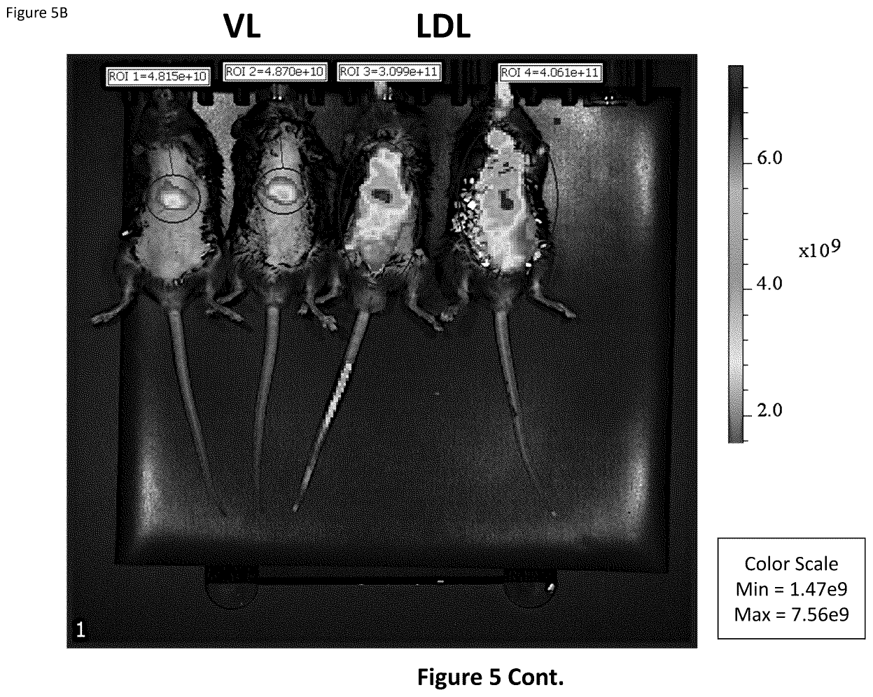

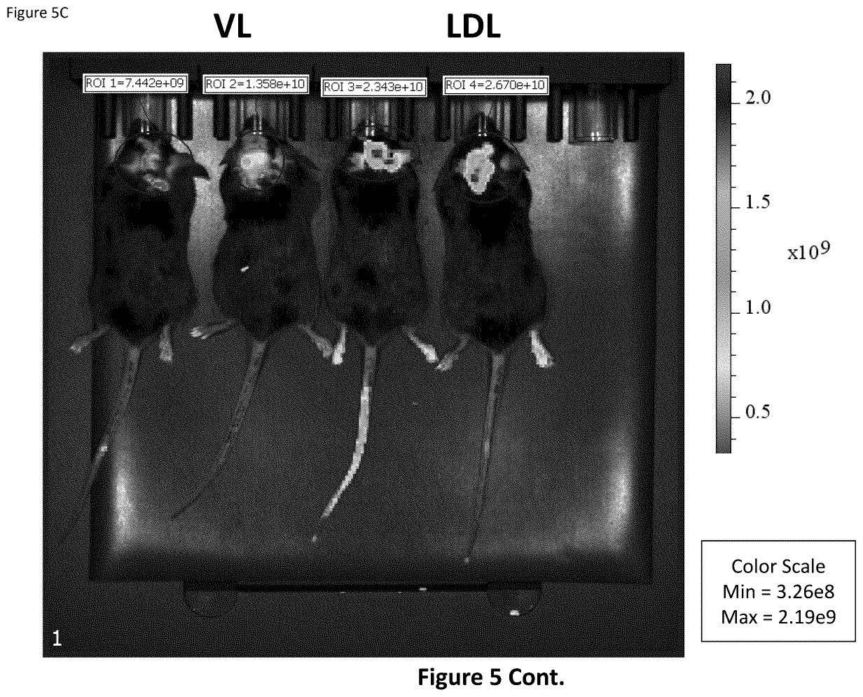

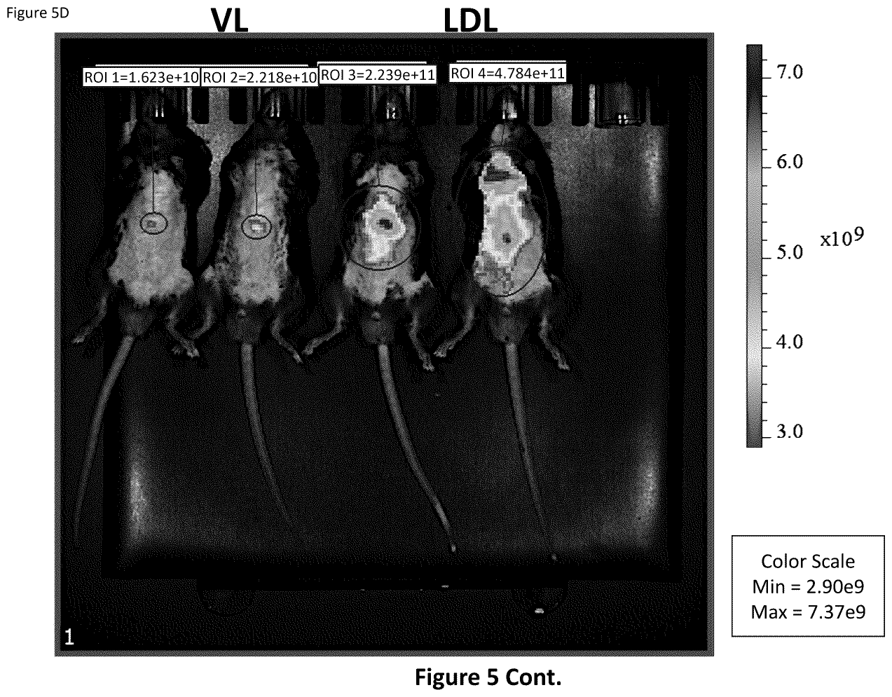

Delivery of LDLs and VLs was repeated. An approximately four-fold increase in dye intensity was once again observed in LDL-treated mice three hours after injection (FIG. 5A). Delivery to the abdomen three hours after injection was approximately 10-fold higher in LDL-treated mice than in VL-treated mice (FIG. 5B). After 24 hours, the dye intensity increase in LDL-treated mice was approximately 2.5-fold higher than VL-treated mice (FIG. 5C) and abdominal delivery was approximately 20-fold higher in LDL-treated mice (FIG. 5D).

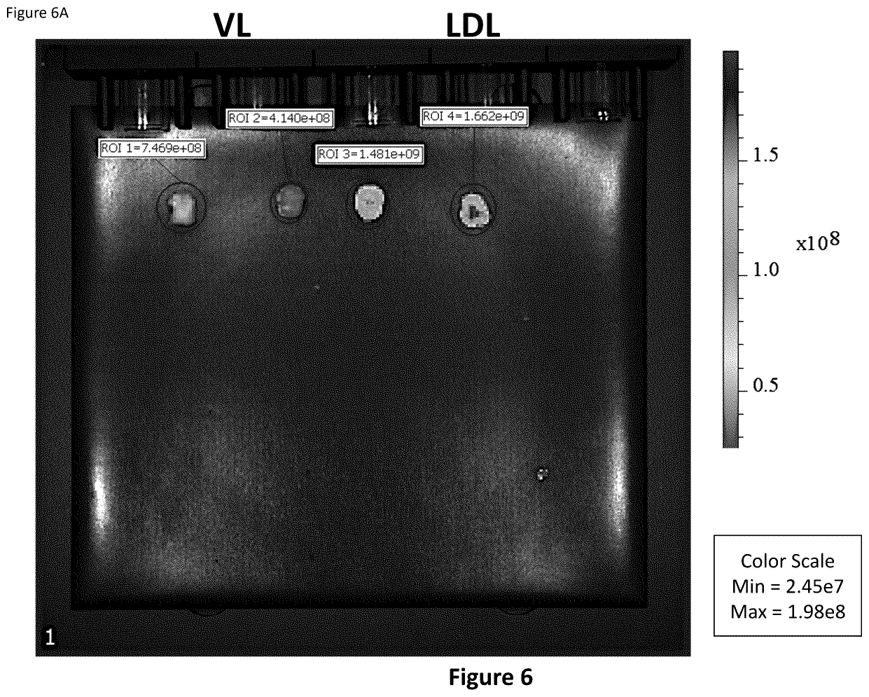

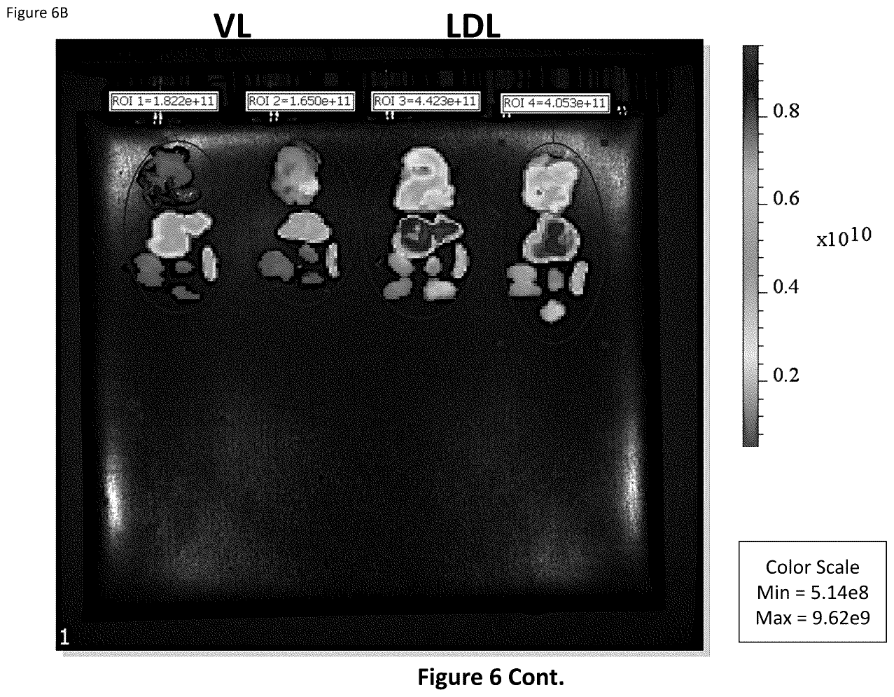

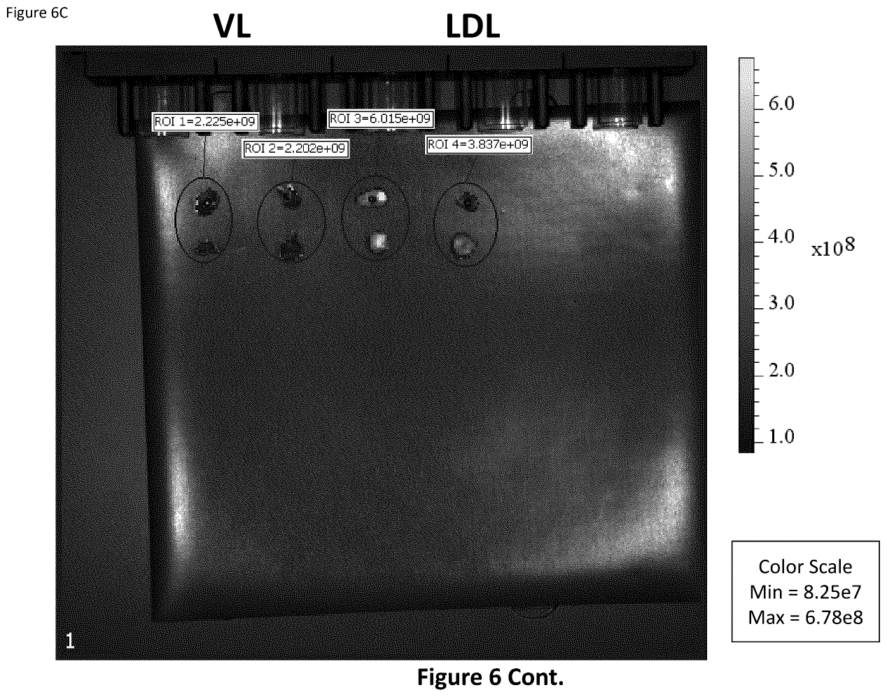

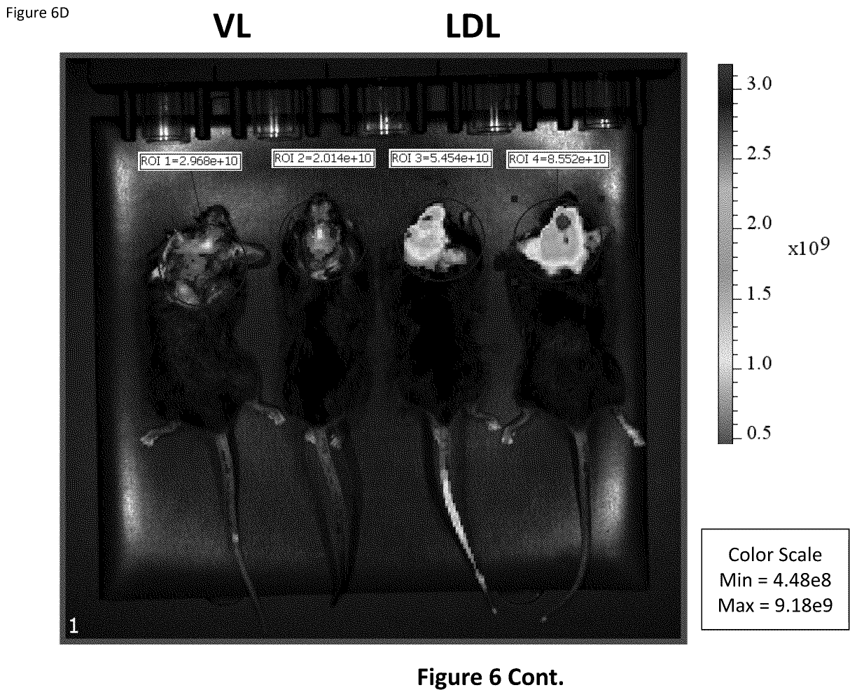

After 24 hours, the mice were sacrificed, hearts were perfused with 1×PBS to remove blood from all organs and tissues, and their organs were harvested. Brains isolated from LDL-treated mice had approximately three-fold greater dye signal intensities than those of VL-treated mice (FIG. 6A). Delivery to the other organs was similarly increased in LDL-treated mice relative to VL-treated mice (FIG. 6B). In the eyes, the delivery of dye by LDLs was approximately 2.5-fold increased relative to VLs (FIG. 6C). After removal of the brain and eyes, dye signal intensity in the body of LDL-treated mice was approximately three-fold increased relative to VL-treated mice (FIG. 6D).

### Example 4: Targeted Delivery in Advanced Age

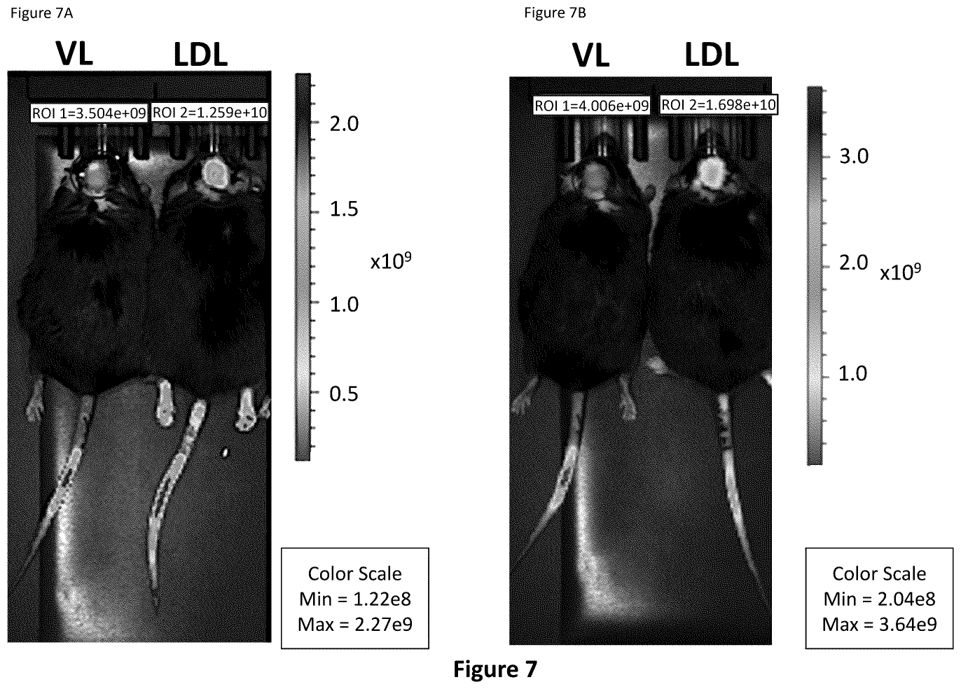

As the majority of neurological diseases and disorders are observed later in life, delivery of targeted liposomes in older animals was examined. Here, mice 17 months of age were injected via tail vein with LDLs or VLs. As in younger mice, after one hour, the dye signal intensity was approximately four-fold higher in LDL-treated mice than in VL-treated mice (FIG. 7A). Unlike younger mice, however, this four-fold increase was maintained after 24 hours (FIG. 7B).

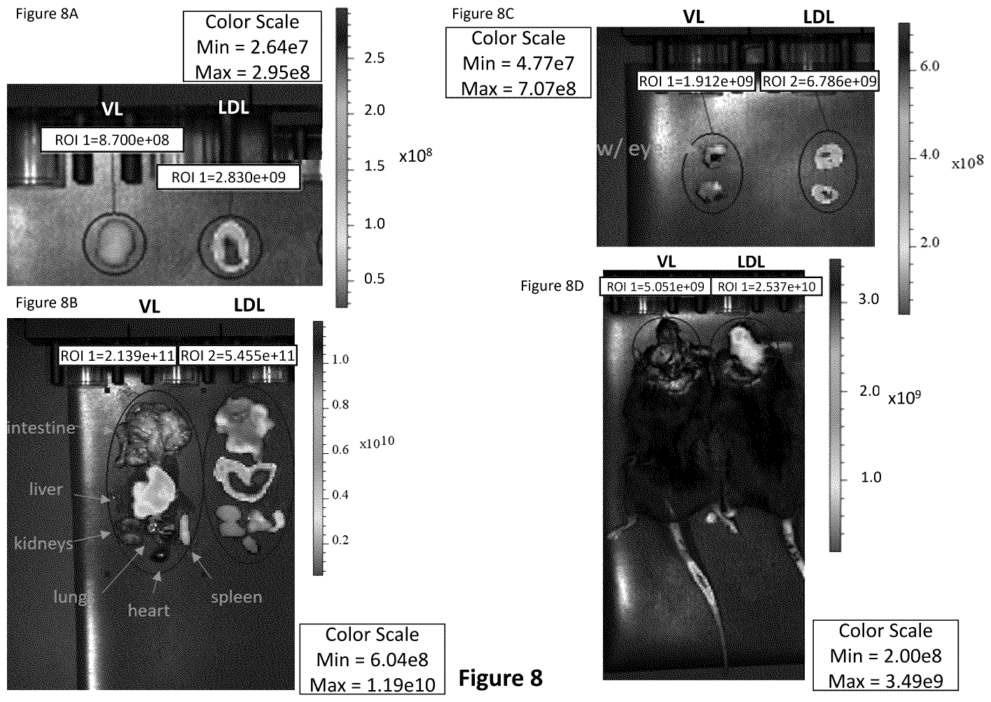

After 24 hours, the mice were sacrificed, hearts were perfused with 1×PBS to remove blood from all organs and tissues, and their organs were harvested. Brains isolated from LDL-treated mice yielded dye signal intensities approximately three-fold greater than those of VL-treated mice (FIG. 8A). Delivery to the other organs was similarly increased in LDL-treated mice relative to VL-treated mice (FIG. 8B). In the eyes, the delivery of dye by LDLs was approximately 3.5-fold increased relative to VLs (FIG. 8C). After removal of the brain and eyes, dye signal intensity in the body of LDL-treated mice was approximately five-fold increased relative to VL-treated mice (FIG. 8D).

In summary, the studies described herein provide novel nanoparticles that cross the BBB and therefore deliver diagnostic and therapeutic agents to the brain.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising at least one ligand that selectively targets major facilitator superfamily domain-containing protein-2a (MFSD2A).

2. The composition of claim 1, wherein the ligand is selected from the group consisting of acyl-carnitine, fatty acid-lysophosphatidylcholine (LysoPC), docosahexaenoic acid LysoPC (DHA-LysoPC), eicosapentaenoic acid LysoPC (EPA-LysoPC), linoleic acid LysoPC (LA-LysoPC), linolelaidic acid LysoPC, tetradecenoyl-carnitine, palmitoyl-carnitine, 3-hydroxypalmitoyl-carnitine, EPA-carnitine, DHA-carnitine, lysophosphatidic acid (LysoPA), lysophosphatidylethanolamine (LysoPE), lysophosphatidylinositol (LysoPI), lysophosphatidylserine (LysoPS), and any combination thereof.

3. The composition of claim 1, wherein the nanoparticle inhibits or reduces the activity of MFSD2A.

4. The composition of claim 1, wherein the nanoparticle selectively targets at least one cell comprising MFSD2A.

5. The composition of claim 4, wherein the at least one cell comprising MFSD2A is an endothelial cell.

6. The composition of claim 5, wherein the endothelial cell is an endothelial cell of a blood brain barrier.

7. The composition of claim 1, wherein the nanoparticle crosses the blood brain barrier, blood retinal barrier, or a combination thereof.

8. The composition of claim 1, wherein the nanoparticle further comprises at least one agent.

9. The composition of claim 8, wherein the agent is encapsulated within, adhered to a surface of, or integrated into the structure of said nanoparticle.

10. The composition of claim 9, wherein the agent is selected from the group consisting of a small molecule, a protein, a nucleic acid molecule, an antibody, a diagnostic agent, an imaging agent, a therapeutic agent, and any combination thereof.

11. The composition of claim 10, wherein the nucleic acid molecule is selected from the group consisting of DNA, cDNA, RNA, mRNA, miRNA, siRNA, modified RNA, microRNA, antagomir, antisense molecule, targeted nucleic acid, CRISPR-Cas9 guide RNA, and any combination thereof.

12-22. (canceled)

23. The composition of claim 1, wherein the composition comprises at least one nanoparticle comprising the at least one ligand; or at least one conjugate comprising the at least one ligand.

24. A method of delivering an agent to a subject in need thereof, the method comprising administering a therapeutically effectively amount of the composition of claim 23 to the subject.

25. The method of claim 24, wherein the agent is selected from the group consisting of a small molecule, a protein, a nucleic acid molecule, an antibody, a diagnostic agent, an imaging agent, a therapeutic agent, and any combination thereof.

26. The method of claim 25, wherein the nucleic acid molecule is selected from the group consisting of DNA, cDNA, RNA, mRNA, miRNA, siRNA, modified RNA, microRNA, antagomir, antisense molecule, targeted nucleic acid, CRISPR-Cas9 guide RNA and any combination thereof.

27. The method of claim 24, wherein the agent is encapsulated within, adhered to a surface of, or integrated into the structure of said nanoparticle, conjugate, or any combination thereof.

28. A method of treating or preventing at least one disease or disorder in a subject in need thereof, the method comprising administering a therapeutically effectively amount of the composition of claim 23 to the subject.

29. The method of claim 28, wherein the disease or disorder is selected from the group consisting of a brain disease or disorder, neurological disease or disorder, cognitive disease or disorder, brain tumor, neurodegenerative disease, food intake disorder, schizophrenia, depression, addiction disease or disorder, and any combination thereof.

30. A method of diagnosing a disease or disorder in a subject in need thereof, the method comprising administering an effectively amount of the composition of claim 23 to the subject,
wherein the composition comprises at least one diagnostic agent.
